SEQUENCE LISTING (42 sequences)

SEQ ID NO: 1 - protein, 258 aa, Cervus elaphus
MNNLNDPPNWNIRPNSRADGGDGSRWNYALLVPMLGLAAFRWIWSRESRKEIEKEREAYRQRTVAFQQDLGARYHATIAESRRAVAHLSLELEKEQNRTTSYREALISQGRKLVEEKKLLEQERAQVLQERRQPLRSAYLRCLGQEEDWQRRARLLLSEFEAALTERQSIYCSLVLPRRRRLELEKSLLVRASTDPVAADLEMAAGLTDIFKHDTHCGDVWNTNKRQNGRLMWLYLRYWELIVELKKFKQVEKAILEK

SEQ ID NO: 2 - protein, 260 aa, Homo sapiens
MNNLNDPPNWNIRPNSRADGGDGSRWNYALLVPMLGLAAFRWIWSRESQKEVEKEREAYRRRTAAFQQDLEAKYHAMISENRRAVAQLSLELEKEQNRTASYREALISQGRKLVEEKKLLEQERAQVMQEKRQVQPLRSAYLSCLQREENWQRRARLLLKEFEAVLTERQNIYCSLFLPRSKRLEIEKSLLVRASVDPVAADLEMAAGLTDIFQHDTYCGDVWNTNKRQNGRLMWLYLKYWELVVELKKFKRVEEAILEK

SEQ ID NO: 3 - protein, 260 aa, Mus musculus
MNNLNDPPNWNIRPNARADGGDGSKWNYALLVPMLGLAAFRWIWSRESQKEIEKARKAYHQRTAAFQQDLEAKYHAVISEHRRAVAQLSLELEKEQNRTSSFREALISQGRKLAEEKKLLEQERAQIKQEKSRLQPLRNVYLSCLQEEDDWQRRAQHVLKEVGEALEERQNIYCSLIIPRSARLELEKSLLVRTSVDPVAADLEMAAGLSDIFKHDKHCGDVWNTNKRQNGKLMWMYLKYWELLVELKKFKKVEKVILEK

SEQ ID NO: 4 - protein, 321 aa, Cervus elaphus
GPVGPSGKDGANGIPGPIGPPGPRGRSGETGPAGPPGNPGPPGPPGPPGPGIDMSAFAGLGQREKGPDPLQYMRADEAAGNLRQHDAEVDATLKSLNNQIESLRSPEGSRKNPARTCRDLKLCHPEWKSGDYWIDPNQGCTLDAMKVFCNMETGETCVYPNPASVPKKNWWSSKSKDKKHIWFGETINGGFHFSYGDDNLAPNTANVQMTFLRLLSTEGSQNITYHCKNSIAYLDEAAGNLKKALLIQGSNDVEIRAEGNSRFTYTVLKDDCTKHTGKWGQTMIEYRSQKTSRLPIIDIAPMDIGGPEQEFGVDIGPVCFL

SEQ ID NO: 5 - protein, 1418 aa, Homo sapiens
MIRLGAPQSLVLLTLLVAAVLRCQGQDVRQPGPKGQKGEPGDIKDIVGPKGPPGPQGPAGEQGPRGDRGDKGEKGAPGPRGRDGEPGTLGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPDPQGATGPLGPKGQTGKPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAEGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGTSGIAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGQQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 6 - protein, 1487 aa, Mus musculus
MIRLGAPQSLVLLTLLIAAVLRCQGQDAQEAGSCLQNGQRYKDKDVWKPSSCRICVCDTGNVLCDDIICEDPDCLNPEIPFGECCPICPADLATASGKLGPKGQKGEPGDIRDIIGPRGPPGPQGPAGEQGPRGDRGDKGEKGAPGPRGRDGEPGTPGNPGPAGPPGPPGPPGLSAGNFAAQMAGGYDEKAGGAQMGVMQGPMGPMGPRGPPGPAGAPGPQGFQGNPGEPGEPGVSGPMGPRGPPGPAGKPGDDGEAGKPGKSGERGLPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGSRGEPGNPGSPGPAGASGNPGTDGIPGAKGSAGAPGIAGAPGFPGPRGPPGPQGATGPLGPKGQAGEPGIAGFKGDQGPKGETGPAGPQGAPGPAGEEGKRGARGEPGGAGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLAGAPGLRGLPGKDGETGAAGPPGPSGPAGERGEQGAPGPSGFQGLPGPPGPPGEGGKQGDQGIPGEAGAPGLVGPRGERGFPGERGSPGAQGLQGPRGLPGTPGTDGPKGAAGPDGPPGAQGPPGLQGMPGERGAAGIAGPKGDRGDVGEKGPEGAPGKDGGRGLTGPIGPPGPAGANGEKGEVGPPGPSGSTGARGAPGEPGETGPPGPAGFAGPPGADGQPGAKGDQGEAGQKGDAGAPGPQGPSGAPGPQGPTGVTGPKGARGAQGPPGATGFPGAAGRVGPPGANGNPGPAGPPGPAGKDGPKGVRGDSGPPGRAGDPGLQGPAGAPGEKGEPGDDGPSGLDGPPGPQGLAGQRGIVGLPGQRGERGFPGLPGPSGEPGKQGAPGASGDRGPPGPVGPPGLTGPAGEPGREGSPGADGPPGRDGAAGVKGDRGETGALGAPGAPGPPGSPGPAGPTGKQGDRGEAGAQGPMGPSGPAGARGIAGPQGPRGDKGESGEQGERGLKGHRGFTGLQGLPGPPGPSGDQGASGPAGPSGPRGPPGPVGPSGKDGSNGIPGPIGPPGPRGRSGETGPVGPPGSPGPPGPPGPPGPGIDMSAFAGLGQREKGPDPMQYMRADEADSTLRQHDVEVDATLKSLNNQIESIRSPDGSRKNPARTCQDLKLCHPEWKSGDYWIDPNQGCTLDAMKVFCNMETGETCVYPNPATVPRKNWWSSKSKEKKHIWFGETMNGGFHFSYGDGNLAPNTANVQMTFLRLLSTEGSQNITYHCKNSIAYLDEAAGNLKKALLIQGSNDVEMRAEGNSRFTYTALKDGCTKHTGKWGKTVIEYRSQKTSRLPIIDIAPMDIGGAEQEFGVDIGPVCFL

SEQ ID NO: 7 - protein, 293 aa, Cervus elaphus
MADDAGAAGGPGGPGGPGMGGRGGFRGGFGSGVRGRGRGRGRGRGRGRGARGGKAEDKEWLPVTKLGRLVKDMKIKSLEEIYLFSLPIKESEIIDFFLGASLKDEVLKIMPVQKQTRAGQRTRFKAFVAIGDYNGHVGLGVKCSKEVATAIRGAIILAKLSIVPVRRGYWGNKIGKPHTVPCKVTGRCGSVLVRLIPAPRGTGIVSAPVPKKLLMMAGIDDCYTSARGCTATLGNFAKATFDAISKTYSYLTPDLWKETVFTKSPYQEFTDHLVKTHTRVSVQRTQAPAVATT

SEQ ID NO: 8 - protein, 293 aa, Homo sapiens [sequence omitted]

SEQ ID NO: 9 - protein, 293 aa, Mus musculus
MADDAGAAGGPGGPGGPGLGGRGGFRGGFGSGLRGRGRGRGRGRGRGRGARGGKAEDKEWIPVTKLGRLVKDMKIKSLEEIYLFSLPIKESEIIDFFLGASLKDEVLKIMPVQKQTRAGQRTRFKAFVAIGDYNGHVGLGVKCSKEVATAIRGAIILAKLSIVPVRRGYWGNKIGKPHTVPCKVTGRCGSVLVRLIPAPRGTGIVSAPVPKKLLMMAGIDDCYTSARGCTATLGNFAKATFDAISKTYSYLTPDLWKETVFTKSPYQEFSDHLVKTHTRVSVQRTQAPAVATT

SEQ ID NO: 10 - protein, 153 aa, Cervus elaphus
KAKKEAPAPPKAEAKAKALKAKKAVLKGVHSHKKKKIRTSPTFRRPKTLRLRRQPKYPRKSAPRRNKLDHYAIIKFPLTTESAMKKIEDNNTLVFIVDVKANKHQIKQAVKKLYDIDVAKVNTLIRPDGEKKAYVRLAPDYDALDVANKIGII

SEQ ID NO: 11 - protein, 156 aa, Homo sapiens [sequence omitted]

SEQ ID NO: 12 - protein, 156 aa, Rattus rattus [sequence omitted]

SEQ ID NO: 13 - protein, 224 aa, Cervus elaphus
AAVRLLSFAKALGAPRPSGTRLSPAPPPRCPRGRSAPPRGRRRRSPRGDRRGCQQNRLLQKWKRSQKRRRERINLQTKKCKQKGKEEQRENRRKWPTKRLKKTCLQKMERLKTRRAQPLMKQKRKKPSLINNHTLSPVSGPCFPSCTIQRNIFINYFVNASFLVALETFLKRRESHLIPFFKCKCFFLRGEIICWVGYFLVQPENSGILDMGGFDCLGCQLNIP

SEQ ID NO: 14 - protein, 168 aa, Homo sapiens
MVLFFRINLQTKKCKQKGKGEQRENRPKWLTKKLKKTYLRKTGKRRLRRVQPLMKQERKKPSLINNHIPCLISGPCLPSCTIQRNIFINYFVNASFLVALETFLRRRESHLIPFFKCKCFFLRGEIICWLFIFWYNQKIVWDIELWEALTVSGVSLTFHRWGVSFYIL

SEQ ID NO: 15 - protein, 215 aa, Cervus elaphus
SEQLVRHFLIETGPKGVKIKGCPSEPYFGSLSALVSQHSISPLSLPCCLRIPSKDPLEEVPEAPVPSNMSTAADLLRQGAACSVLYLTSVETESLTGPQAVARASSAALSCSPRPTPAVVHFKVSAQGITLTDNQRKLFFRRHYPVNSITFSSTDPQDRRWTNSDGTTSKIFGFVAKKPGSPWENVCHLFAELDPDQPAGAIVTFITKVLLGQRK

SEQ ID NO: 16 - protein, 1285 aa, Homo sapiens
MERRWDLDLTYVTERILAAAFPARPDEQRHRGHLRELAHVLQSKHRDKYLLFNLSEKRHDLTRLNPKVQDFGWPELHAPPLDKLCSICKAMETWLSADPQHVVVLYCKGNKGKLGVIVSAYMHYSKISAGADQALATLTMRKFCEDKVATELQPSQRRYISYFSGLLSGSIRMNSSPLFLHYVLIPMLPAFEPGTGFQPFLKIYQSMQLVYTSGVYHIAGPGPQQLCISLEPALLLKGDVMVTCYHKGGRGTDRTLVFRVQFHTCTIHGPQLTFPKDQLDEAWTDERFPFQASVEFVFSSSPEKIKGSTPRNDPSVSVDYNTTEPAVRWDSYENFNQHHEDSVDGSLTHTRGPLDGSPYAQVQRPPRQTPPAPSPEPPPPPMLSVSSDSGHSSTLTTEPAAESPGRPPPTAAERQELDRLLGGCGVASGGRGAGRETAILDDEEQPTVGGGPHLGVYPGHRPGLSRHCSCRQGYREPCGVPNGGYYRPEGTLERRRLAYGGYEGSPQGYAEASMEKRRLCRSLSEGLYPYPPEMGKPATGDFGYRAPGYREVVILEDPGLPALYPCPACEEKLALPTAALYGLRLEREAGEGWASEAGKPLLHPVRPGHPLPLLLPACGHHHAPMPDYSCLKPPKAGEEGHEGCSYTMCPEGRYGHPGYPALVTYSYGGAVPSYCPAYGRVPHSCGSPGEGRGYPSPGAHSPRAGSISPGSPPYPQSRKLSYEIPTEEGGDRYPLPGHLASAGPLASAESLEPVSWREGPSGHSTLPRSPRDAPCSASSELSGPSTPLHTSSPVQGKESTRRQDTRSPTSAPTQRLSPGEALPPVSQAGTGKAPELPSGSGPEPLAPSPVSPTFPPSSPSDWPQERSPGGHSDGASPRSPVPTTLPGLRHAPWQGPRGPPDSPDGSPLTPVPSQMPWLVASPEPPQSSPTPAFPLAASYDTNGLSQPPLPEKRHLPGPGQQPGPWGPEQASSPARGISHHVTFAPLLSDNVPQTPEPPTQESQSNVKFVQDTSKFWYKPHLSRDQAIALLKDKDPGAFLIRDSHSFQGAYGLALKVATPPPSAQPWKGDPVEQLVRHFLIETGPKGVKIKGCPSEPYFGSLSALVSQHSISPISLPCCLRIPSKDPLEETPEAPVPTNMSTAADLLRQGAACSVLYLTSVETESLTGPQAVARASSAALSCSPRPTPAVVHFKVSAQGITLTDNQRKLFFRRHYPVNSITFSSTDPQDRRWTNPDGTTSKIFGFVAKKPGSPWENVCHLFAELDPDQPAGAIVTFITKVLLGQRK

SEQ ID NO: 17 - protein, 303 aa, Homo sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 18 - protein, 136 aa, Cervus elaphus
RRRSRMEIPVPVQPSWLRRASAPLPGLSAPGRLFDQRFGEGLLEAELAALCPAALAPYYLRAPSVALPTAQVSTDPGHFSVLLDVKHFSPEEIAVKVVGDHVEVHARHEERPDEHGYIAREFTRLPLAAGVDPAAV

SEQ ID NO: 19 - protein, 160 aa, Homo sapiens
MEIPVPVQPSWLRRASAPLPGLSAPGRLFDQRFGEGLLEAELAALCPTTLAPYYLRAPSVALPVAQVPTDPGHFSVLLDVKHFSPEEIAVKVVGEHVEVHARHEERPDEHGFVAREFHRRYRLPPGVDPAAVTSALSPEGVLSIQAAPASAQAPPPAAAK

SEQ ID NO: 20 - protein, 162 aa, Rattus norvegicus
MEIRVPVQPSWLRRASAPLPGFSTPGRLFDQRFGEGLLEAELASLCPAAIAPYYLRAPSVALPTAQVPTDPGYFSVLLDVKHFSPEEISVKVVGDHVEVHARHEERPDEHGFIAREFHRRYRLPPGVDPAAVTSALSPEGVLSIQATPASAQASLPSPPAAK

SEQ ID NO: 21 - protein, 363 aa, Cervus elaphus
GDRGQKGHRGFTGLQGLPGPPGPNGEQGSAGIPGPFGPRGPPGPVGPSGKEGSPGPLGPIGPPGVRGSVGEAGPEGPPGEPGPPGPPGPPGHLTAALGDIMGHYDESMPDPLPEFTEDQAAPDDKNKTDPGVHATLKSLSSQIETMRSPDGSRKHPARTCDDLKLCHSAKQSGEYWIDPNQGSAEDAIKVYCNMETGETCISANPSSVPRKTWWASKSPDNKPVWYGLDMNRGSQFVYGDHQSPNAAITQMTFLRLLSKEASQNITYICKNSVGYMDDQTKNLKKAVVLKGSNDLEIKAEGNVRFRYIVLHDSCSKRNGNVGKTIFEYRTQNVARLPIIDLAPVDVGSTDQEFGIEIGPVCFV

SEQ ID NO: 22 - protein, 1496 aa, Homo sapiens
MMANWAEARPLLILIVLLGQFVSIKAQEEDEDEGYGEEIACTQNGQMYLNRDIWKPAPCQICVCDNGAILCDKIECQDVLDCADPVTPPGECCPVCSQTPGGGNTNFGRGRKGQKGEPGLVPVVTGIRGRPGPAGPPGSQGPRGERGPKGRPGPRGPQGIDGEPGVPGQPGAPGPPGHPSHPGPDGLSRPFSAQMAGLDEKSGLGSQVGLMPGSVGPVGPRGPQGLQGQQGGAGPTGPPGEPGDPGPMGPIGSRGPEGPPGKPGEDGEPGRNGNPGEVGFAGSPGARGFPGAPGLPGLKGHRGHKGLEGPKGEVGAPGSKGEAGPTGPMGAMGPLGPRGMPGERGRLGPQGAPGQRGAHGMPGKPGPMGPLGIPGSSGFPGNPGMKGEAGPTGARGPEGPQGQRGETGPPGPVGSPGLPGAIGTDGTPGPKGPTGSPGTSGPPGSAGPPGSPGPQGSTGPQGNSGLPGDPGFKGEAGPKGEPGPHGIQGPIGPPGEEGKRGPRGDPGTLGPPGPVGERGAPGNRGFPGSDGLPGPKGAQGERGPVGSSGPKGSQGDPGRPGEPGLPGARGLTGNPGVQGPEGKLGPLGAPGEDGRPGPPGSIGIKGQPGTMGLPGPKGSNGDPGKPGEAGNPGVPGQRGAPGKDGKVGPYGPPGPPGLRGERGEQGPPGPTGFQGHPGPPGPPGEGGKPGDQGVPGGPGAVGPLGPRGERGNPGERGEPGITGLPGEKGMAGGHGPDGPKGSPGPSGTPGDTGPPGLQGMPGERGIAGTPGPKGDRGGIGEKGAEGTAGNDGAGGLPGPLGPPGPAGLLGEKGEPGPRGLVGPPGSRGNPGSRGENGPTGAVGFAGPQGSDGQPGVKGEPGEPGQKGDAGSPGPQGLAGSPGPHGPNGVPGLKGGRGTQGPPGATGFPGSAGRVGPPGPAGAPGPAGPLGEPGKEGPPGPRGDPGSHGRVGVRGPAGPPGGPGDKGDPGEDGQPGPDGPPGPAGTTGQRGIVGMPGQRGERGMPGLPGPAGTPGKVGPTGATGDKGPPGPVGPPGSNGPVGEPGPEGPAGNDGTPGRDGAVGERGDRGDPGPAGLPGSQGAPGTPGPVGAPGDAGQRGDPGSRGPIGHLGRAGKRGLPGPQGPRGDKGDHGDRGDRGQKGHRGFTGLQGLPGPPGPNGEQGSAGIPGPFGPRGPPGPVGPSGKEGNPGPLGPLGPPGVRGSVGEAGPEGPPGEPGPPGPPGPPGHLTAALGDIMGHYDESMPDPLPEFTEDQAAPDDKNKTDPGVHATLKSLSSQIETMRSPDGSKKHPARTCDDLKLCHSAKQSGEYWIDPNQGSVEDAIKVYCNMETGETCISANPSSVPRKTWWASKSPDNKPVWYGLDMNRGSQFAYGDHQSPNTAITQMTFLRLLSKEASQNITYICKNSVGYMDDQAKNLKKAVVLKGANDLDIKAEGNIRFRYIVLQDTCSKRNGNVGKTVFEYRTQNVARLPIIDLAPVDVGGTDQEFGVEIGPVCFV

SEQ ID NO: 23 - protein, 1497 aa, Mus musculus
MMANWVGARPLLILSVLLGYCVSIKAQEQENDEYDEEIACTQHGQMYLNRDIWKPSPCQICVCDNGAILCDKIECPEVLNCANPITPPGECCPVCPQTGGGDTSFGRGRKGQKGEPGLVPVVTGIRGRPGPAGPPGSQGPRGDRGPKGRPGPRGPQGIDGEPGMPGQPGAPGPPGHPSHPGPDGMSRPFSAQMAGLDEKSGLGSQVGLMPGSVGPVGPRGPVGLQGQQGGAGPAGPPGEPGEPGPMGPIGSRGPEGPPGKPGEDGEPGRNGNTGEVGFSGSPGARGFPGAPGLPGLKGHRGHKGLEGPKGEIGAPGAKGEAGPTGPMGAMGPLGPRGMPGERGRLGPQGAPGKRGAHGMPGKPGPMGPLGIPGSSGFPGNPGMKGERGPHGARGPEGPQGQRGETGPPGPAGSQGLPGAVGTDGTPGRKGATGSAGTSGPPGLAGPPGSPGPQGSTGPQGIRGQSGDPGVPGFKGEAGPKGEPGPHGIQGPIGPPGEEGKRGPRGDPGTVGPPGPMGERGAPGNRGFPGSDGLPGPKGAQGERGPVGSSGPKGGQGDPGRPGEPGLPGARGLTGNPGVQGPEGKLGPLGAPGEDGRPGPPGSIGIRGQPGSMGVPGPKGSSGDLGKPGEAGNAGVPGQRGAPGKDGEVGPSGPVGPPGLAGERGEAGPPGPTGFQGLPGPPGPPGEGGKAGDQGVPGEPGAVGPLGPRGERGNPGERGEPGITGLPGEKGMAGGHGPDGPKGNPGPTGTIGDTGPPGLQGMPGERGIAGTPGPKGDRGGIGEKGAEGTAGNDGARGLPGPLGPPGPAGLLGAPGEPGPRGLVGPPGSRGNPGSRGENGPTGAVGFAGPQGSDGQPGVKGEPGEPGQKGDAGSPGPQGLAGSPGPHGPHGVPGLKGGRGTQGPPGATGFPGSAGRVGPPGPAGAPGPAGPAGEPGKEGPPGLRGDPGSHGRVGDRGPAGPPGSPGDKGDPGEDGQPGPDGPPGPAGTTGQRGIVGMPGQRGVTGMPGLPGPAGTPGKVGPTGATGDKGPPGPVGPPGSNGPVGEPGPEGPAGNDGTPGRDGAVGERGDRGDPGPAGLPGSQGAPGTPGPVGAPGDAGQRGEPGSRGPVGPPGRAGKRGLPGPQGPRGDKGDNGDRGDRGQKGHRGFTGLQGLPGPPGPNGEQGSAGIPGPFGPRGPPGPVGSSGKEGNPGPLGPIGPPGVRGSVGEAGPEGPPGEPGPPGPPGPPGHLTAALGDIMGHYDENMPDPLPEFTEDQAAPDDTNKTDPGIHVTLKSLSSQIETMRSPDGSKKHPARTCDDLKLCHPTKQSGEYWIDPNQGSAEDAIKVYCNMETGETCISANPASVPRKTWWASKSPDNKPVWYGLDMNRGSQFTYGDYQSPNTAITQMTFFRLLSKEASQNLTYICRNTVGYMDDQAKNLKKAVVLKGSNDLEIKGEGNIRFRYTVLQDTCSKRNGNVGKTIFEYRTQNVARLPIIDVGPVDIGNADQEFGLDIGPVCFM

SEQ ID NO: 24 - protein, 420 aa, Cervus elaphus
PGAPGAPGAPGPVGPAGKSGDRGETGPAGPAGPIGPVGARGPAGPQGPRGDKGETGEQGDRGIKGHRGFSGLQGPPGPPGSPGEQGPSGASGPAGPRGPPGSAGTPGKDGLNGLPGPIGPPGPRGRTGDAGPAGPPGPPGPPGPPGPPSGGYDLSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPIVAQKNWYISKNPKDKRHVWYGESMTGGFQFEYGGQGSDPADVAIQLTFLRLMSTEASQNITYHCKNSVAYMDQQTGNLKKALLLQGSNEIEIRAEGNSRFTYSVTYDGCTSHTGAWGKTVIEYKTTKTSRLPIIDVAPLDVGAPDQEFGFDVGPVCFL

SEQ ID NO: 25 - protein, 1461 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKTSRLPIIDVAPLDVGAPDQEFGFDVGPVCFL

SEQ ID NO: 26 - protein, 1453 aa, Mus musculus
MFSFVDLRLLLLLGATALLTHGQEDIPEVSCIHNGLRVPNGETWKPEVCLICICHNGTAVCDDVQCNEELDCPNPQRREGGCCAFCPEEYVSPNSEDVGVEGPKGGPGPQGPRGPVGPPGRDGIPGQPGLPGPPGPPGPPGPPGLGGNFASQMSYGYDEKSAGVSVPGPMGPSGPRGLPGPPGAPGPQGFQGPPGEPGEPGGSGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGPPGTAGARGNDGAVGAAGPPGPTGPTGPPGFPGAVGAKGEAGPQGARGSEGPQGVRGEPGPPGPAGAAGPAGNPGADGQPGAKGANGAPGIAGAPGFPGARGPSGPQGPSGPPGPKGNSGEPGAPGNKGDTGAKGEPGATGVQGPPGPAGEEGKRGARGEPGPSGLPGPPGERGGPGSRGFPGADGVAGPKGPSGERGAPGPAGPKGSPGEAGRPGEAGLPGAKGLTGSPGSPGPDGKTGPPGPAGQDGRPGPAGPPGARGQAGVMGFPGPKGTAGEPGKAGERGLPGPPGAVGPAGKDGEAGAQGAPGPAGPAGERGEQGPAGSPGFQGLPGPAGPPGEAGKPGEQGVPGDLGAPGPSGARGERGFPGERGVQGPPGPAGPRGNNGAPGNDGAKGDTGAPGAPGSQGAPGLQGMPGERGAAGLPGPKGDRGDAGPKGADGSPGKDGARGLTGPIGPPGPAGAPGDKGEAGPSGPPGPTGARGAPGDRGEAGPPGPAGFAGPPGADGQPGAKGEPGDTGVKGDAGPPGPAGPAGPPGPIGNVGAPGPKGPRGAAGPPGATGFPGAAGRVGPPGPSGNAGPPGPPGPVGKEGGKGPRGETGPAGRPGEVGPPGPPGPAGEKGSPGADGPAGSPGTPGPQGIAGQRGVVGLPGQRGERGFPGLPGPSGEPGKQGPSGSSGERGPPGPMGPPGLAGPPGESGREGSPGAEGSPGRDGAPGAKGDRGETGPAGPPGAPGAPGAPGPVGPAGKNGDRGETGPAGPAGPIGPAGARGPAGPQGPRGDKGETGEQGDRGIKGHRGFSGLQGPPGSPGSPGEQGPSGASGPAGPRGPPGSAGSPGKDGLNGLPGPIGPPGPRGRTGDSGPAGPPGPPGPPGPPGPPSGGYDFSFLPQPPQEKSQDGDRYYRADDANVVRDRDLAVDATLKSLSQQIENIRSPEGSRKNPARTCRDLKMCHSDWKSGEYWIDPNQGCNLDAIKVYCNMETGQTCVFPTQPSVPQKNWYISPNPKEKKHVWFGESMTDGFPFEYGSEGSDPTDVAIQLTFLRLMSTEASQNITYHCKNSVAYMDQQTGNLKKALLLQGSNEIELRGEGNSRFTYSRVVDGCTSHTGTWGKTVIEYKTTKTSRLPIIDVAPLDIGAPDQEFGLDIGPACFV

SEQ ID NO: 27 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 28 - DNA, 1426 bp, Cervus elaphus
ctggccccgtcggtccctctggcaaagatggtgctaatggaatccctggccccattggacctcctggaccccgtggacgttctggcgagactggccctgctggtcctcctggaaaccctggaccccctggccctcctggcccccccggtcctggcattgacatgtctgcctttgctggcctcggccagagagagaagggccccgaccccctgcagtacatgcgggccgatgaggcagccggcaacctgagacagcatgatgccgaggtggacgccacactcaagtccctcaacaaccagatcgagagcctccgcagccccgagggctcacgcaagaacccagctcgcacctgccgagacctgaaactctgccaccctgagtggaagagcggagactactggatcgaccccaaccagggctgcaccctggatgccatgaaggttttctgcaacatggagactggcgagacctgcgtctaccccaacccagccagtgttcccaagaagaactggtggagcagcaagagcaaggacaagaaacacatctggtttggagaaaccatcaacggtggcttccatttcagctatggagatgacaacctggctcccaacaccgccaacgtccagatgaccttcctccgcctgctgtccaccgagggctcccagaacatcacctaccactgcaagaacagcattgcctacctggacgaagctgctggcaacctcaagaaggctctgctcatccagggctccaacgacgtggagatccgggctgagggcaacagcaggttcacatacaccgttctgaaggatgactgcacgaaacacaccggtaagtggggccagactatgatcgagtaccggtcacagaagacctcacgtctccccatcattgacattgcacccatggacataggagggcccgagcaggaattcggtgtggacatagggcctgtctgcttcttgtaaaaacccgaacccagaaccaacacaatccattgcaaacccaaaggacccaagtactttccaatcccagtcactctaggactctgcactgaatggctgatctgacctgacgcccattcatcccacccgctcacagtctggactttgctcccctctctaagagacctgaactgggcagactgcaaaataaaatctcggtgttctatttatttattgtcttcctgtaagacctttgggtcaaggcagagacaggaaactaactggtgtgagtcaaatgccccctgagtgactgccccccagcccaggcaagaggcccccctgcaggtgccgggcgcgggaactgtgtgtgtcctacacaatggtgctattctgtgtcaaacacctctgtattttttaagacgtcaattgatattaaaaacaaaaaaattattggaaagtaaaaaaaaaaaaaaaaaaaaaaa

SEQ ID NO: 29 - DNA, 957 bp, Cervus elaphus
ccaccaaatggcggatgacgccggtgctgcgggagggcccggaggcccggggggccctggaatgggaggccgcggtggcttccgcggaggcttcggtagtggcgtccggggccggggtcgtggccgcggtcggggccggggcagaggccgcggagctcgcggaggcaaggccgaggacaaggagtggctccccgttaccaagctgggccgcctggtcaaggacatgaagatcaagtccctggaggagatctaccttttctctctgcccatcaaggagtctgagattattgacttttttctgggagcgtccctcaaggatgaagttttgaagattatgcccgtgcaaaagcagacccgtgctggccagcggaccaggttcaaggcatttgttgccatcggggattacaatggacatgtcggtctgggtgtcaagtgctccaaggaagtagccactgccatccgtggggccatcatcctggctaagctgtccatcgtccccgtgcgaaggggctactgggggaacaagatcggcaagccccacacggttccttgcaaggtgactggccgctgtggctccgtgctggtgcgcctcatccctgcccccagaggcactggcatcgtctccgcccctgtgcccaagaagctgctgatgatggccggcatcgacgactgctacacttctgccaggggctgcaccgccaccctgggcaacttcgccaaggccacttttgatgccatttccaagacctacagttacctcactcctgacctctggaaagagacggtgttcaccaagtctccatatcaggaatttactgaccatcttgtgaagacccacaccagagtctccgtgcagaggacccaggccccagctgtagccaccacataattttataacataattttacaaagagaataataaagtgaatgaaaccggaaaaaaaaaaaaaaaaaaaa

SEQ ID NO: 30 - DNA, 532 bp, Cervus elaphus
cgaaggcgaagaaggaagcccctgcccctcctaaagctgaagccaaagcaaaggctttgaaggccaagaaagcagtgttgaaaggtgtccacagccacaagaaaaagaagatccggacgtcacccaccttccggcggcccaaaacactgcggctcaggaggcagcccaaatatcctcggaagagcgcccccaggagaaacaaacttgaccactatgccatcatcaaattccccctcaccactgagtcagccatgaagaaaatagaagacaacaacacactggtattcattgtggatgtcaaggccaacaagcaccaaattaaacaggctgtgaagaagctctatgacattgacgtggctaaggtcaatactctgatcaggcctgatggagagaagaaggcatatgttcgactggctcctgactatgatgctttggatgttgccaacaaaattgggatcatctaaactgagtccagctggctaattccaaatataagttttcactatgtaaaaaaaaaaaaaaaaaaaaaaaa

SEQ ID NO: 31 - DNA, 1224 bp, Cervus elaphus
ggcagcggtcaggctactcagcttcgcgaaggctctcggcgcgccgcggccctcaggcacccggctctcgcccgccccgccgccacgatgcccaagaggaaggtcagctccgccgagggggcggcgaaggaggagcccaagaggagatcggcgaggttgtcagcaaaaccggctcctgcaaaagtggaaacgaagccaaaaaaggcggcgggaaaggataaatcttcagacaaaaaagtgcaaacaaaagggaaaagaggagcaaagggaaaacaggcggaagtggccaaccaagagactaaagaagacttgcctgcagaaaatggagagactaaaaacgaggagagcccagcctctgatgaagcagaagagaaagaagccaagtctgattaataaccacacactcagtcctgtcagtggtccctgtttcccttcttgtacaatccagaggaatatttttatcaactattttgtaaatgcaagttttttagtagctctagaaacatttttaaaaaggagggaatcccacctcatcccattttttaagtgtaaatgcttttttttaagaggtgaaatcatttgctgggttggttattttttggtacaaccagaaaatagtgggatattggatatgggaggctttgattgtcttgggtgtcaacttaacattccttagatggggggagcttttatatcctataatacaaaagcatactaaatggcagtttggagtcagttgtgcatttaatgtcttgaacactttaaattacttctcttcccattttgttttggtagaattatttcctacagcaaaccactttttgatcttggctctcctggtcagaattttgtgcactatactataacatctttggtcgtggtagtccagttttcctagtaacttggttaatgtgctgtgaacgattgacagtttgggtatgtagtgtatatgatattaaattgtgaatcagtgggacttatgatgtaacaacatatcaatatttgaagatattggtacttgatatcctgttaaggaaagttgctccaaattttaagctggaaagtcactggaataactgttaagaatcacaactacatgatattttagatttctggtacgtatgtgaagaattgtgtaccaattgaaatatctgtgtagtgatcctcaaaacaaccaataaaatctccgttataaaagaaaaaaaaaaaaaaaaaaaaaaaaaa

SEQ ID NO: 32 - DNA, 1163 bp, Cervus elaphus
tcggaacagctggtccgccattttctcattgagactgggcccaaaggggtgaagatcaagggctgtcccagcgagccctactttggcagcctatcagccctggtctcccagcactccatctccccactgtccctgccctgctgcctgcgcattcccagcaaagatcctctggaggaggtcccagaggccccagtgcccagcaacatgagtacggcagcagacctcctgcgtcagggcgccgcctgcagtgtgctctacctgacctcagtggagacggagtcgctgacgggcccccaagcggtggcacgggccagctccgcggctctgagctgcagcccccgccccacgccagccgttgtccacttcaaggtctcagcccagggcatcacactcacagacaaccaaaggaagctcttctttcgccgccattatccagtgaacagcatcaccttctccagcactgaccctcaggaccggagatggaccaactccgacgggaccacctccaagatctttggtttcgtggccaagaagccgggaagcccttgggagaatgtgtgtcacctctttgcagagcttgacccagatcagcctgcaggcgccattgtcaccttcatcaccaaagttttactgggccagaggaaatgaaggaaggccacaagctccaagcccgcgtcaacactgtgcccctctcagcaccacacagccctcacttcccctggcctggacccaggagacccaggagccgcctctcccctaggaatggggagcagacacaccggcctgcaacactgctctccttccccgcccccagcctgctaagcaagtggatgggcccatgagatgaccttgcatgtgagcagagggcagagacgggtgtgtgagggtgaggtggtggagcctggaaggggtgatccagacagccccacctgcaggagagcgtcagcgctggcaggggagacaggccttgcctgctccaccagctgcaggtcccagcacggcagggagagaggagaggtgtggggagcaaggcactccctcctctgcctcccctctgagcagagagatcagagtaggatcacatgaaaacgggggggaaaaaagagtctatttttgtctaataataaagagtttctataatgtttaaaaaaaaaaaaaaaaaa

SEQ ID NO: 33 - DNA, 1474 bp, Cervus elaphus
cgctcagggcacctggtcggcgagttcccggccggaggtgtatctccatgaataacttaaatgaccccccaaattggaatatccggcccaattccagggctgatggaggtgatggaagcagatggaattatgccctgttggttccaatgctgggactagctgcttttcgctggatttggtctcgggagtcgagaaaagaaatagaaaaggagagagaagcgtaccgtcagaggacggttgccttccagcaggaccttggagccaggtaccatgccacaattgcagaaagccggcgggccgtggcacacttgtccctggaacttgaaaaggagcagaacagaacaactagttaccgagaagccctcatctctcaggggcgcaagttggtggaagagaagaaacttctggaacaggagcgggctcaggtcctgcaggagaggaggcagcccttgcggagtgcgtacctgcgctgcctgggccaggaggaggactggcagcgcagggccaggctcctgctgagcgagttcgaggccgcgctcaccgagagacagagcatctactgcagcctggtgctcccgcgccgcaggcggctcgagctcgagaagagcttgctggtccgcgcgtccactgacccggtggccgcagacctcgagatggcagctggcctcactgacatatttaagcacgatacgcactgtggtgacgtctggaacaccaacaagcgccaaaacgggaggctcatgtggctgtatctcagatactgggaactaatcgttgaactgaagaagtttaagcaggtagagaaagccatactggaaaagtaagacaggagtgaacggctccaggtcagagtcatgggttgtgggttttccgatgttcgctgctcctcctgccagcgctccctagttgtgaccgtgcatgcacaccgccacctcttagcagcggccattcccgtcaccctctgaggaagacagcaaggcctctgtcccctgcagcagctaaggacacagtctcagaagcaggtcaatattttattaagcaggacaggataacctcatagctttagagtaaaattgtttttaagaatatcaaatacagtgttcaccctataagtcattctgtcacttcctaaataagttctgttttctcctcaaattatttttctctctcctaaaactacagttagaagttgtcaggtagcggtgaggactgcctcacagatgggaacagacggtggtgacgccagcaaggtttcgtggtctgaatcccatcagtgtttcttttttccacttgataacccttgtgggtgtttggagtttgctgtgcctgtattcagtaagcagatactgtttatttaggttggtgcaaaggtaattgtggttttgcattgttgaattttgccatttgatattgaaatacattcttaaataaatgaagttatgttttgaaagtgcaaaaaaaaaaaaaaaaaaaa

SEQ ID NO: 34 - DNA, 1088 bp, Cervus elaphus
cattaacggtgctaaacatagacattttaacccaagtcacgacattcttagctgtaactcagctatcacggcctcttgctcacccactaatggtcccattttccccttgccgtgtgcacctctgcccattgtcttggtggcacatgggtggaacacttgatctgctcgagtctgccttcaacacacgttgcatcttcagattttctacttttctgcttgaaactaatattcaccaagtcagactttgtgttaactttatttcagggtattggctgccagggggtcattcctaagtggcctgaagatggacaaagggaagtaacaggcacgtgatgttggcaaggatgcttctagggctagaggatcagtggtgggagagacctgcagaatctaccagccagaaccgcagataacaaatcttgtggtcaggggctgtgactgagagaaggaaattgaggctgtgttctggaagtacatataaacttctcacacaaacccagttcttcaccatttccctttctcactttgcagtgccatttctttttgcattaggcaatttgctcagacttttcagagccacggcccatccgttctctggaatcccccacacctctgagaggtggatcaccacatcctgcagggctgctcccctccaaactacctttcggagatgcaggacagggaggctgtttcagccagaaagaccaaaatcaagagcgaggtgcagaacgtggtaaaacagaaaaagggcaggtggcaaattggttttcttttgggttttctggttttttttttttccacatctggatggctgtcaccagagatctttccttcagtcgctagcatgttcctcctcttctcccctccccactttttctttctattaatcaaaagaaatttcaaaatcaatgggatggtcggatctcacaggctgagaactcgttcacctccaagcatttcatgaaaaagctgcttcttattaatcatgcaaactcttgccacgatgtgaagagtttgacaaatctttcaaaataaaaagtactgatttagaaactgaaaaaaaaaaaaaaaaaaaaaaaaaaaaaaaa

SEQ ID NO: 35 - DNA, 410 bp, Cervus elaphus
cgacggcggagcaggatggagatcccggtgcctgttcagccgtcttggctgcgccgcgcctcggcccctttgcctgggctgtcggctcccgggcgcctcttcgaccagcgcttcggcgaggggctgctggaggccgagctggctgcgctctgccctgccgcgctggccccctactacctgcgcgcacccagcgtggcgctgcctaccgcccaggtatcgaccgaccccgggcatttctcggtgctgctggatgtgaaacacttctcacccgaggaaattgccgtcaaggtggttggtgaccacgtggaggttcatgcgcgccacgaggagcgcccggatgagcacggatacattgcgcgcgagttcacgcggctaccgcttgccgctggcgtggaccctgcggccgtgac

SEQ ID NO: 36 - DNA, 588 bp, Cervus elaphus
  misc_feature (157)..(157): N=unknown
tcatcccctcaccccatttcaatcccacccaccaccaaagattatggtgtaggcaagccctgcccccaccctaggccagtcaagcataatccccccttctcagatgtccaagacccgtgcacagacctcctaccccggaccatcctggcctggtccncaagactggatccttcccctcattccaaccagatacacttctcctcaccctctcccttcaacccattctctaacctgaaacctcagccagccactcccagatccttgaaccccttttctgaccctacccgtgtacccctattctaagccaaccagaaccctcaacctcaaactgtatagatacccatccctcctccccagagtctgcacagatatcccacgctatccagaactcctcagtcactctgtcttgaccccccaaatctccaaccacaccacccctccccttattctccaagacccaaccaagcagccactttctttaattccctacaatctttctccctcctcaaattccctgatgccccatccccccacctaggcccactcccccaataaatgtgctagagctaaaaaaaaaaaaaaaaaaaa

SEQ ID NO: 37 - DNA, 1625 bp, Cervus elaphus
aggggatcgaggtcagaagggtcacagaggctttactggtcttcaaggtcttcctggacctcctggtccaaatggtgaacaaggcagtgctggaatccctggaccatttggcccaagaggccccccaggtccagttggtccttcaggcaaagaaggaagccctgggccgcttgggcccattgggcctcctggtgtgcggggcagcgttggagaagcaggccctgagggtcctcctggtgagcctggtccccccggccctccgggaccccctggccaccttacagctgctcttggggatatcatggggcactatgatgagagcatgccagacccacttccggagtttactgaagatcaggcggctcctgatgacaaaaacaaaaccgaccccggggtacatgcgaccctgaagtcactcagtagtcagattgaaaccatgcgtagccctgatggctctagaaagcaccctgcccggacctgtgacgacttaaagctttgccattctgcaaagcagagcggtgagtactggattgaccctaaccagggatctgctgaagatgcaatcaaagtttactgcaacatggaaacaggagaaacgtgtatttcagcaaatccatccagtgtcccacggaaaacctggtgggccagcaaatctcctgataataagcctgtttggtatggtcttgatatgaatcgaggatctcagtttgtttatggagaccaccagtcacctaatgcagccattactcagatgaccttcttgcgccttttatcgaaagaagcctcccagaacatcacctacatctgtaaaaacagtgtaggatacatggatgatcaaactaagaacttgaagaaagctgtggttctcaaagggtcaaatgacttagaaatcaaagcagagggaaatgttagattcagatacatagttcttcatgattcttgctctaaacgaaatggaaacgtgggcaagaccatctttgaatatagaacacagaatgtggcacgcttgcccatcatagatcttgcccctgtggatgttggcagtacagaccaagaatttggcatagaaattggaccagtttgttttgtgtaaagcaagccgagatacatcgacaatgagcaccacccctaccatcagtgaccaccaccattcacaagactttgactgtttgaagctgatcctgagactcttgaagtaatggctgattctgcatcagcattgtatatatggtcttaagtgcctggcctccttatccttcagaatatttattttacttacagtcctcaagttttaattgatttaaaatatttttcaatacaacagtttaggtttaaaatgatcaatgacaaagaccaccttttaaaaaaaaagtaaactgattgaataaataaatctccgttttcttcatttcagtgtaatgacaaagttgcttagtatttatgagaaaaactttcttcctggcagatagcttaaagagtggggtatataaaatcacaacacttttatttcacgtggctgcaattggaaaaatacaaagtaatgcccttttgtgacctctcatttacagattatcaattaaaaatgaaatcaaaatgtgaaaaaaaaaaaaaaaaaaaaaaaaaaa

SEQ ID NO: 38 - DNA, 1508 bp, Cervus elaphus
cctggtgctcctggcgctcccggtgcccccggccctgtcggacctgctggcaagagcggtgatcgtggtgagactggtcctgctggtcctgctggtcccattggccccgttggtgcccgtggccccgctggaccccaaggcccccgtggtgacaagggtgaaacaggcgaacagggcgacagaggcattaagggtcaccgtggcttctctggtctccagggtccccctggccctcccggctctcctggtgagcaaggtccttccggagcctctggtcctgctggtccccgcggtccccctggctctgctggtactcctggcaaagatggactcaatggtctcccaggccccatcggtccccctgggcctcgaggtcgcactggtgatgctggtcctgctggtcctcccggccctcctggaccccctggtccccccggtcctcccagcggcggctacgacttaagcttcctgccccagccacctcaagagaaggctcacgatggtggccgctactaccgggctgatgatgccaatgtggtccgtgaccgtgacctcgaggtggacaccaccctcaagagcctgagccagcagatcgagaacatccggagccctgaaggcagccgcaagaaccccgcccgcacctgccgtgacctcaagatgtgccactctgactggaagagcggagaatactggattgaccccaaccaaggctgcaacctggatgccattaaggtcttctgcaacatggaaactggtgagacctgtgtgtaccccactcagcccatcgtggcccagaagaactggtacatcagcaagaaccccaaggacaagaggcacgtctggtacggcgagagcatgaccggcggattccagttcgagtacggcggccagggctccgatcctgccgatgtggccatccagctgactttcctgcgcctgatgtccaccgaggcctcccagaacatcacctaccactgcaagaacagcgtggcctacatggaccagcagactggcaacctcaagaaggccctgctcctccagggctccaacgagatcgagatccgggccgagggcaacagccgcttcacctacagcgtcacctacgacggctgcacgagtcacaccggagcctggggcaagacagtgatcgaatacaaaaccaccaagacctcccgcttgcccatcatcgatgtggcccccttggacgttggcgccccagaccaggaattcggcttcgacgttggccctgtctgcttcctgtaaactccttccaccccaacctggctccctcccacccaacccacttgcccctgactctggaaacagacaaacaacccaaaccgaaacccccaaaaagccaaaaaatgggagacaatttcacatggactttggaaaatatttttttcctttgcattcatctctcaaacttagtttttatctttgaccaactggacatgaccaaaaaccaaaagtgcattcaaccttaccaaaaaaaaaaaaaaaaaaaa

SEQ ID NO: 39 - DNA, 20 bp, Artificial sequence
  Oligonucleotide primer
gttccacacgtcaccacagt

SEQ ID NO: 40 - DNA, 24 bp, Artificial sequence
  Oligonucleotide primer
cgtatcgtgcttaaatatgtcagt

SEQ ID NO: 41 - protein, 13 aa, Dama dama
  MISC_FEATURE (10)..(10): X=L or I
FVEGXYXVEXDTK

SEQ ID NO: 42 - protein, 6 aa, Dama dama
  MISC_FEATURE (3)..(3): X=L or I
EGXYXV

(19) United States
(12) Patent Application Publication
(10) Pub. No.: US 2004/0157329 A1
(43) Pub. Date: Aug. 12, 2004

(54) Matrix gene expression in chondrogenesis

(76) Inventors: Rebecca Roubin, Summer Hill (AU); Peter Ghosh, Fairlight (AU)

Correspondence Address: BOZICEVIC, FIELD & FRANCIS LLP, 200 MIDDLEFIELD RD, SUITE 200, MENLO PARK, CA, 94025, US

(21) Appl. No.: 10/468,091

(22) PCT Filed: Feb. 15, 2002
(86) PCT No.: PCT/AU02/00163

(30) Foreign Application Priority Data
Feb. 15, 2001 (AU) PR 3116

(51) Int. Cl. C12N 15/85

(52) U.S. Cl. 435/455; 435/325

(57) ABSTRACT

The present invention is directed to polypeptides which can be used to stimulate cell growth and/or division. More particularly, the present invention is directed to polypeptides which can be used to stimulate mesenchymal cell growth and/or division. The invention also relates to a method for transfecting chondrocytes and other mesenchymal cells with vectors carrying genes capable of stimulating chondrogenesis, osteogenesis, growth, repair, regeneration and/or restoration of the cellular matrix.

### TECHNICAL FIELD

The present invention relates to polypeptides which stimulate cell growth and/or division. More particularly, the present invention relates to polypeptides which stimulate mesenchymal cell growth and/or division. The invention also relates to a method for transfecting chondrocytes and other mesenchymal cells with vectors carrying genes capable of stimulating chondrogenesis, osteogenesis, growth, repair, regeneration and/or restoration of the extracellular matrix.

### BACKGROUND ART

In the developing embryo, chondrogenesis commences with the proliferation, migration and condensation of mesenchymal stem cells into zones which are destined-to become-specific regions of the skeleton. This morphogenetic phase is followed by differentiation of mesenchymal-derived cells and the expression by these differentiated cells of matrix proteins characteristic of the tissues they occupy (FIG. 1). In the growing limb bud, chondrocytes become the predominant cell type and at a specific stage selectively express genes required to form a cartilaginous matrix. The most abundant matrix gene produced in cartilage is type II collagen (Cancedda et al. 1995; Sandell et al. 1991; Muratoglu et al. 1995) which is co-expressed with the large aggregating proteoglycan, aggrecan. The cartilaginous anlage produced by these cells during chondrogenesis is eventually transformed into the long bones of foetal and post-foetal life by a process of endochondral ossification. This involves the progressive proliferation, maturation, hypertrophy and apoptosis of chondrocytes followed by mineralisation of the lacunae vacated by the chondrocyte, vascular invasion and proliferation of osteoblasts and the deposition of a bone matrix (FIG. 2). The bone lengthens longitudinally by the progressive proliferation of chondrocytes followed by the replacement of cartilage by vascularised bone. In the late stages of foetal development and after birth this process takes place in the growth plate where calcification, chondrocyte death, osteoblastogenesis and vascular invasion lead to the formation of bone-trabeculae at the interface between the hypertrophic/dying chondrocytes of the cartilage.

All these important cellular events are tightly regulated by genomic, paracrine, autocrine, endocrine and mechanical factors. The identity and the respective roles these factors perform, however, are still largely unknown. Type II collagen is the major structural protein of the cartilage matrix representing approximately 50% of the dry weight of the tissues. This collagen provides the structural scaffold of the matrix, maintaining the overall shape of the cartilage and entrapping the macromolecular hydrated proteoglycan aggregate (aggrecan) within its network Type II collagen also undergoes ionic, hydrophobic and hydrogen bonding with other matrix molecules such as type IX collagen, fibronectin, osteonectin, hyaluronan and the dermatan sulphate containing proteoglycans, decorin and biglycan. The proteoglycan aggregate, aggrecan because of its high anionic charge and water binding capacity confer the resilience and viscoelastic properties to the tissue necessary for its mechanical functions. The relative distribution of proteoglycans and type II collagen in human foetal cartilage at sites of endochondral ossification as well as the formation of bone are shown in FIG. 2.

In contrast to all other species, the antlers of the deer family undergo an annual shedding and regeneration throughout their adult life. The process of antler formation requires the rapid seasonal growth of cartilage from periosteal tissues on the pedicles of the skull with the progressive transformation of the cartilage to bone via endochondral ossification in the distal regions and endochondral ossification and membranous bone formation at the proximal margins (Banks and Newbrey, 1983; Goss, 1983; Kierdorf et al. 1995). The rates of cartilage growth and ossification are unparalleled in the adult vertebrate kingdom (up to 2 cm/week). While there are many morphological and histological similarities between the processes of cartilage conversion to bone in the antler and the epiphyseal growth plate Banks and Newbrey, 1983; Goss, 1983; Kierdorf et al. 1995), there are also differences, particularly in the distal cartilage region which exhibits characteristics of the early stage of cartilage formation (chondrogenesis) in utero FIG. 2) (Banks and Newbrey, 1983; Goss, 1983; Kierdorf et al. 1995; Price et al. 1996). In the distal region of the developing antler (FIGS. 3 and 4) the columbic assembly of chondrocytes is more diffuse than in the epiphyseal growth plate and the non-mineralised cartilaginous zone maybe sub-divided morphologically into an outermost tip of mesenchymal cell zone which merges into a prechondroblastic zone which is penetrated by blood vessels. In the chondroblastic zone located proximally to the prechondroblastic region the cells show typical chondrocyte morphology but with a hypertrophic appearance in the deeper regions (FIG. 4). This zone is also served by vascular channels but the extracellular matrix still stains strongly for type II collagen and proteoglycans (FIG. 4) which are characteristic gene products of hyaline cartilage. In addition to type II collagen, type I and type III collagens, which are absent from normal growth plate cartilage are reported to be present in the cartilaginous tip of antler (Newbrey et al. 1983).

The two alternatively spliced gene transcripts, type IIA procollagen and IIB are also expressed in the cartilaginous tip of regenerating antler (Price et al. 1996). However, only type IIA procollagen, the isoform which is considered to induce chondrogenesis, was transiently expressed in the chondroprogenitor region of cartilage (Price et al. 1996). Collectively; these reports suggest that the process of chondrogenesis and ossification in the developing deer antler resembles more closely the pattern of long bone formation in early foetal tissue rather than in post-foetal cartilage; however it may be considered as a hybrid of the two.

Degenerative and traumatic injuries to cartilage and other weight bearing connective tissues such as the intervertebral disc, meniscus and tendon are very common but often difficult to treat medically. For example, the injury to diarthrodial joints can be sufficiently intense as to cause chondral or osteochondral fractures, while disc and tendon rupture leads to cell necrosis, neurological and vascular deficits, which apart from impairment of function are accompanied by long-term morbidity. If the injury to connective tissues such as joint cartilage penetrates into the subchondral bone (osteochondral defects) imperfect healing in the form of fibrocartilage formation can occur. This type of repair is mechanically inferior to the original tissue and can fail under everyday stress loading. When the injury to the connective tissue is confined to the avascular regions, healing rarely occurs spontaneously (Buckwalter et al. 1987). The process of cartilage, disc or other connective tissue injury may also be exacerbated in older subjects where cell numbers and their viability ability to respond to growth factors may already be diminished (Loeser et al., 2000; Hashimoto et al. 1998). It is common in these instances that more progressive cartilage or disc degeneration follows leading to overload of subchondral bone in adjacent supporting structures and the onset of osteoarthritis (OA). Thus it is generally agreed by those skilled in the art that it is the avascularity and the end stage differentiation of connective tissue cells which precludes their normal regeneration and repair following injury. Furthermore, this situation is exacerbated by the low density, diminished responsiveness and viability of cells within the connective tissues resulting from the aging process, altered hormonal status, mechanical factors and impaired nutrition. These deficiencies are of considerable significance since failure of connective tissue function, as occurs with OA of the peripheral joints and spine, are the most frequent cause of pain and disability in all societies and represents the most common rheumatic disorder worldwide.

Within recent years, attempts have been made to promote tissue regeneration and repair in cartilage, meniscus, tendon and the intervertebral disc by a variety-of methods. Some of the approaches employed are described in recent publications on this subject (Buckwalter and Mankin, 1998; Breinan et al. 1998; Wakitani et al. 1998, Rahfoth et al. 1998; Nishida et al. 2000; Moon et al. 2000). In the Buckwalter and Mankin (1998) article the authors conclude that “None of the current procedures for repairing or transplanting articular cartilage restores a normal articular surface, but they can decrease symptoms associated with chondral defects in some patients”. A common method for undertaking cartilage repair is to use autologous transplantation of chondrocytes (supported by an artificial matrix) into the chondral defects. Clinical reports suggest that this surgery is effective in repairing small defects in younger patients (Brittberg et al. 1994; Peterson, 1996) but the procedure is still far from satisfactory due to the inherent limited proliferative and biosynthetic capacity of the mature chondrocyte for the reasons already cited. As discussed, attempts to overcome this problem by breaching the subchondral plate by drilling or fenestration to allow undifferentiated mesenchymal cells of the bone marrow to penetrate and occupy the defect have also only been partially successful. The material that initially occupies these defects invariably deteriorates to fibrocartilage which, by its very nature, is incapable of performing the specialised biomechanical functions required of articular cartilage (Nehrer et al. 1999). Nehrer and co-workers (1999) showed that cells which repaired a chondral defect in rabbit joints expressed low transcription levels of the type II collagen gene due to insufficient differentiation of mesenchymal cells of the bone marrow to the chondrocyte phenotype. From these findings they conclude that it was the absence of sufficient amounts of fundamentally important regulatory factors, or progenitor cells, in the repair tissue which inhibited its transformation to normal cartilage.

In more recent years attempts to overcome some of these problems has led to the utilisation of the technique of transfecting connective tissue cells grown in a compatible biomatrix with growth factor genes or other genes which could promote regeneration or decrease turnover of the extracellular matrix. Examples of this approach include: Cultured equine articular chondrocytes, mesenchymal stem cells, synovial explants, and synovial intimal cells were transfected with an E1-deleted adenoviral vector containing equine insulin-like growth factor-I coding sequence. (Nixon et al. 2000). Discs injected with Ad/CMV-hTGFβ1 exhibited extensive and intense positive immunostaining for transforming growth factor β1 with the nucleus pulposus showing a 30-fold increase in active transforming growth factor β1 production. Furthermore, tissues so transfected synthetised 100% more proteoglycan relative to non transfected control tissue (Nishida et al. 1999). The use of gene transfer of antiinflammatory cytokines or the in vivo induction of their expression has been described as a potential method for the treatment of osteoarthritis by decreasing matrix degradation (Fernandes et al. 2000). Others researchers have used monolayer cultures of bovine chondrocytes seeded onto polylactic acid (PLA), polyglycolic acid (PGA), collagen matrices to induce the production of collagen type I, collagen type II, and aggrecan. The collagen type I gene was upregulated on collagen scaffolds throughout the culture period but PLA and PGA showed initial induction followed by downregulation (Saldanha and Grande, 2000). Bone morphogenic protein-7 is a member of a family of 16 related BMPs of the TGF-β superfamily. While the major site of action of BMPs is thought to be bone, it has also been shown to have effectiveness in cartilage repair by stimulating synthesis of type II collagen and aggrecan in human articular chondrocytes when administered as a gene-enhanced tissue within a biomatrix into the defects (Mason et al. 2000).

It is clear from the existing art that repair of defects within avascular connective tissue has been largely confined to the transplantation into the defect of biomatrices seeded with host cells transfected with growth factor or cytokine/anti-cytokine genes which are normally expressed by the those cells but at a reduced level in their non-transfected state. The rational for such an approach is that the amount of extracellular matrix synthesised by the cell will be increased in the transfected cells thereby filling the defect and supporting repair or alternatively transfecting them with genes which diminish the rate at which the matrix produced by the cell is catabolised. While such approaches may provided some benefit, none have exploited the inherent genetic information for growth; repair, regeneration and/or restoration which already exists within the target cells and which was once expressed during foetal development and growth but, because of the advanced state of differentiation and maturation of those cells, may no longer be expressed.

### DISCLOSURE OF INVENTION

The present inventors-have identified polypeptides that are expressed in high levels in growing/dividing cells. Accordingly, the present invention provides for the use of these polypeptides in stimulating cell growth and/or division.

Thus, in a first aspect, the present invention provides a method of stimulating cell growth and/or division, the method comprising contacting, or inserting into, an animal cell a polypeptide comprising a sequence selected from the group consisting of:

a) a sequence as shown in SEQ ID NO:1,

b) a sequence as shown in SEQ ID NO:2,

c) a sequence as shown in SEQ ID NO:3, and

d) a sequence which is at least 50% identical to any one of (a) to (c).

In a preferred embodiment, the polypeptide is at least 60%, more preferably at least 70%, more preferably at least 80%, even more preferably at least 90%, even more preferably at least 95%, even more preferably at least 97%, and most preferably at least 99% identical to any one of (a) to (c).

In another aspect, the present invention provides a method of stimulating cell growth and/or division, the method comprising contacting, or inserting into, an animal cell a polypeptide comprising a sequence selected from the group consisting of:

a) a sequence as shown in SEQ ID NO:4,

b) a sequence as shown in SEQ ID NO:5,

c) a sequence as shown in SEQ ID NO:6, and

d) a sequence which is at least 70% identical to any one of (a) to (c).

In a preferred embodiment, the polypeptide is at least 80%, even more preferably at least 90%, even more preferably at least 95%, even more preferably at least 97%, and most preferably at least 99% identical to any one of (a) to (c).

In another aspect, the present invention provides a method of stimulating cell growth and/or division, the method comprising contacting, or inserting into, an animal cell a polypeptide comprising a sequence selected from the group consisting of:

a) a sequence as shown in SEQ ID NO:7,

b) a sequence as shown in SEQ ID NO:8,

c) a sequence as shown in SEQ ID NO:9, and

d) a sequence which is at least 80% identical to any one of (a) to (c).

In a preferred embodiment, the polypeptide is at least 90%, even more preferably at least 95%, even more preferably at least 97%, and most preferably at least 99% identical to any one of (a) to (c).

In another aspect, the present invention provides a method of stimulating cell growth and/or division, the method comprising contacting, or inserting into, an animal cell a polypeptide comprising a sequence selected from the group consisting of:

a) a sequence as shown in SEQ ID NO:10,

b) a sequence as shown in SEQ ID NO:11,

c) a sequence as shown in SEQ ID NO:12, and

d) a sequence which is at least 85% identical to any one of (a) to (c).

In a preferred embodiment, the polypeptide is at least 90% o, even more preferably at least 95%, even more preferably at least 97%, and most preferably at least 99% identical to any one of (a) to (c).

In another aspect, the present invention provides a method of stimulating cell growth and/or division, the method comprising contacting, or inserting into, an animal cell a polypeptide comprising a sequence selected from the group consisting of:

a) a sequence as shown in SEQ ID NO:13,

b) a sequence as shown in SEQ ID NO:14, and

c) a sequence which is at least 70% identical to any one of (a) or (b).

In a preferred embodiment, the polypeptide is at least 80%, even more preferably at least 90%, even more preferably at least 95%, even more preferably at least 97%, and most preferably at least 99% identical to any one of (a) or (b).

In another aspect, the present invention provides a method of stimulating cell growth and/or division, the method comprising contacting, or inserting into, an animal cell a polypeptide comprising a sequence selected from the group consisting of:

a) a sequence as shown in SEQ ID NO:15,

b) a sequence as shown in SEQ ID NO:16, and

c) a sequence which is at least 50% identical to any one of (a) or (b).

In a preferred embodiment, the polypeptide is at least 60%, more preferably at least 70%, more preferably at least 80%, even more preferably at least 90%, even more preferably at least 95%, even more preferably at least 97%, and most preferably at least 99% identical to any one of (a) or (b).

In another aspect, the present invention provides a method of stimulating cell growth and/or division, the method comprising contacting, or inserting into, an animal cell a polypeptide comprising a sequence selected from the group consisting of:

a) a sequence as shown in SEQ ID NO:17, and

b) a sequence which is at least 60% identical to a).

In a preferred embodiment, the polypeptide is at least 70%, more preferably at least 80%, even more preferably at least 90%, even more preferably at least 95%, even more preferably at least 0.97%, and most preferably at least 99% identical to a).

In another aspect, the present invention provides a method of stimulating cell growth and/or division, the method comprising contacting, or inserting into, an animal cell a polypeptide comprising a sequence selected from the group consisting of:

a) a sequence as shown in SEQ ID NO:18,

b) a sequence as shown in SEQ ID NO:19,

c) a sequence as shown in SEQ ID NO:20, and

d) a sequence which is at least 50% identical to any one of (a) to (c).

In a preferred embodiment, the polypeptide is at least 60%, more preferably at least 70%, more preferably at least 80%, even more preferably at least 90%, even more preferably at least 95%, even more preferably at least 97%, and most preferably at least 99% identical to any one of (a) to (c).

In another aspect, the present invention provides a method of stimulating cell growth and/or division, the method comprising contacting, or inserting into, an animal cell a polypeptide comprising a sequence selected from the group consisting of:

a) a sequence as shown in SEQ ID NO:21,

b) a sequence as shown in SEQ ID NO:22,

c) a sequence as shown in SEQ ID NO:23, and

d) a sequence which is at least 65% identical to any one of (a) to (c).

In a preferred embodiment, the polypeptide is at least 70%, more preferably at least 80%, even more preferably at least 90%, even more preferably at least 95% o, even more preferably at least 97%, and most preferably at least 99% identical to any one of (a) to (c).

In another aspect, the present invention provides a method of stimulating cell growth and/or division, the method comprising contacting, or inserting into, an animal cell a polypeptide comprising a sequence selected from the group consisting of:

x) a) a sequence as shown in SEQ ID NO:24,

b) a sequence as shown in SEQ ID NO:25,

c) a sequence as shown in SEQ ID NO:26, and

d) a sequence which is at least 75% identical to any one of (a) to (c).

In a preferred embodiment, the polypeptide is at least 80%, even more preferably at least 9096, even more preferably at least 95%, even more preferably at least 97%, and most preferably at least 99% identical to any one of (a) to (c).

In another aspect, the present invention provides a method of stimulating cell growth and/or division, the method comprising contacting, or inserting into, an animal cell a polypeptide comprising a sequence selected from the group consisting of:

a) a sequence as shown in SEQ ID NO:27, and

b) a sequence which is at least 35% identical to a).

The increased cell division and/or matrix gene expression by chondrogenesis may result from the action of transthyretin.

In a preferred embodiment, the polypeptide is at least 40%, more preferably at least 50%, more preferably at least 60%, more preferably at least 70%, more preferably at least 80%, even more preferably at least 90%, even more preferably at least 95%, even more preferably at least 97%, and most preferably at least 99% identical to (a).

In a preferred embodiment of all previous aspects, the cell is a somatic cell. More preferably, the somatic cell is a mesenchymal cell. More preferably, the mesenchymal cell is selected from the group consisting of: chondrocytes and osteocytes.

In a preferred embodiment of all previous aspects, the polypeptide is provided by introducing into the cell an expression vector encoding the polypeptide.

In a further preferred embodiment of all previous aspects, the cell is removed from an animal, preferably a mammal, cultured in vitro, transformed or transfected with a polynucleotide encoding the polypeptide and then placed back into an animal.

In this regard, and in a particularly preferred embodiment, the present invention provides a method of stimulating chondrogenesis, cartilage, disc or connective tissue growth, repair, regeneration and/or restoration in an animal, the method comprising transfecting a chondrocyte or other mesenchymal cell from an animal with a polynucleotide encoding the polypeptide, and transplanting said transformed chondrocyte or other mesenchymal cell into the animal at a suitable site such that, at said site, the polynucleotide molecule is expressed in the chondrocyte or other mesenchymal cell thereby causing chondrogenesis, cartilage, disc or connective tissue growth, repair, regeneration and/or restoration in the animal.

The cell may be removed from the animal (e.g. a human), transfected and then placed in the animal, preferably at the site where chondrogenesis, cartilage, disc or connective tissue growth, repair, regeneration and/or restoration is required in the animal.

One example of this embodiment comprises the use of a 1.5 kb full length cDNA prepared from clone DACC-7 according to standard techniques which is cloned into a vector such as pBK-CMV.2 (as described herein) and transfected into chondrocytes according to the method described by Goomer et al. (2000) where it was observed that lapine chondrocytes grown in pellet culture showed enhanced proliferation as determined by the higher incorporation of the radioactive precursor, ³H-thymidine, into DNA produced by these cells (FIG. 6). These pellet culture keep the chondrocyte phenotype as shown by Goomer et al. (2000) even though they are proliferating.

In a further preferred embodiment of all previous aspects, the cell is transformed or transfected in vivo with a polynucleotide encoding the polypeptide.

In this regard, and in a particularly preferred embodiment, the present invention provides a method of stimulating chondrogenesis, cartilage, disc or connective tissue growth, repair, regeneration and/or restoration in an animal, the method comprising transfecting in vivo a chondrocyte or other mesenchymal cell in an animal (see U.S. Pat. No. 6,159,464 and Goomer et al. 2000) with a polynucleotide encoding the polypeptide, such that the polynucleotide molecule is expressed in the chondrocyte or other mesenchymal cell thereby causing chondrogenesis, cartilage, disc or connective tissue growth, repair, regeneration and/or restoration in the animal.

In another aspect, the present invention provides a method of inhibiting cell growth and/or division, the method comprising contacting, or inserting into, an animalcell a compound which hybridizes to, and inhibits the translation of, a polynucleotide encoding a polypeptide as outlined in the previous aspects.

In another aspect, the present invention provides a method of identifying an agent that modulates the activity of a polypeptide that stimulates animal cell growth and/or division, the method comprising

i) exposing the polypeptide to a candidate agent, and

ii) assessing the ability of the candidate agent to modulate the ability of the polypeptide to stimulate cell growth and/or division,

wherein the polypeptide is a polypeptide as outlined in the previous aspects.

In one embodiment, the agent inhibits the ability of the polypeptide to stimulate cell growth and/or division.

In another embodiment, the agent enhances the ability of the polypeptide to stimulate cell growth and/or division.

In a particularly preferred embodiment of all previous aspects, the animal cell is a mammalian cell. More preferably, the mammalian cell is a human cell.

In a further aspect, the present invention provides a method of stimulating mesenchymal cell growth and/or division, the method comprising exposing animal mesenchymal cells to conditioned media, or an active fraction thereof, obtained from deer antler cartilage cells.

The conditioned media can be obtained from any culture in which deer antler cartilage cells are grown in vitro. One example, as exemplified herein is growing the deer antler cartilage cells in DMEM:F12/10%(v)FBS.

As used herein, the term “active fractions thereof” refers to at least partially purified portions of the conditioned media that maintain the factor(s) which stimulate mesenchymal cell growth and/or division.

Preferably, the deer antler cartilage cells are selected from the group consisting of: prechondrocytes, mature chondrocytes, hypertropic chondrocytes, or a combination thereof.

Preferably, the method further comprises exposing the cells to a growth factor. More preferably, the growth factor is selected from the group consisting of: insulin-like growth factor (IGF-1), TGF-beta, fibroblast growth factor (FGF), vascular endothelial growth factor (VEGF), morphogenic bone factors, thyroid hormones (thyroxine), parathyroid hormone related protein (PTHrP), sex hormones, luteinizing hormone (LH) and prolactin.

The present inventors unexpectedly determined that chondrocytes of rapidly growing cartilage of regenerating deer antler express unique genes which are not expressed in mature articular cartilage chondrocytes or chondrocytes of the epiphyseal growth plate as observed on Northern Blot analysis of deer chondrocyte mRNA Of even greater surprise was the finding that some of these gene transcripts are also expressed in the early stage of chondrogenesis in the human foetal tissues as demonstrated by in-situ hybridisation (the results of which are provided hereinafter).

Accordingly, in another aspect the present invention provides an isolated polynucleotide molecule comprising a nucleotide sequence encoding_a gene product expressed in chondrocytes of rapidly growing cartilage of regenerating deer antler.

Preferably, the novel gene product is one which is also expressed in the early stage of chondrogenesis in human foetal tissue and in human chondrocytes and like cells attempting to restore the extracellular matrix and thus functionality of degenerate and osteoarthritic cartilages.

In a further aspect, the present invention provides a substantially purified polypeptide comprising a sequence selected from the group consisting of:

a) a sequence as shown in SEQ ID NO:1, and

b) a sequence which is at least 91% identical to a),

wherein the polypeptide is capable of stimulating animal cell growth and/or division.

Preferably, the polypeptide is at least 95% identical to a). More preferably, the polypeptide is at least 99% identical to a).

In another aspect, the present invention provides a substantially purified polypeptide comprising a sequence selected from the group consisting of:

a) a sequence as shown in SEQ ID NO:4, and

b) a sequence which is at least 99% identical to a),

wherein the polypeptide has a biological activity selected from the group consisting of: stimulating animal cell growth and/or division, or a structural component of extracellular matrix.

In another aspect, the present invention provides a substantially purified polypeptide comprising a sequence selected from the group consisting of:

a) a sequence as shown in SEQ ID NO:7, and

b) a sequence which is at least 99% identical to a),

wherein the polypeptide has a biological activity selected from the group consisting of: stimulating animal cell growth and/or division, or a subunit involved in protein synthesis.

In another aspect, the present invention provides a substantially purified polypeptide comprising a sequence selected from the group consisting of:

a) a sequence as shown in SEQ ID NO:13, and

b) a sequence which is at least 90% identical to a),

wherein the polypeptide has a biological activity selected from the group consisting of: stimulating animal cell growth and/or division, or altering chromatin structure.

Preferably, the polypeptide is at least 9596 identical to a). More preferably, the polypeptide is at least 99% identical to a).

In another aspect, the present invention provides a substantially purified polypeptide comprising a sequence selected from the group consisting of:

a) a sequence as shown in SEQ BD NO:15, and

b) a sequence which is at least 99% identical to a),

wherein the polypeptide has a biological activity selected from the group consisting of: stimulating animal cell growth and/or division, or regulating cell migration.

In another aspect, the present invention provides a substantially purified polypeptide comprising a sequence selected from the group consisting of:

a) a sequence as shown in SEQ ID NO:18, and

b) a sequence which is at least 91% identical to a),

wherein the polypeptide has a biological activity selected from the group consisting of: stimulating animal cell growth and/or division, or responses to cell stress.

Preferably, the polypeptide is at least 95% identical to a). More preferably, the polypeptide is at least 99% identical to a).

In another aspect, the present invention provides a substantially purified polypeptide comprising a sequence selected from the group consisting of:

a) a sequence as shown in SEQ ID NO:21, and

b) a sequence which is at least 96% identical to a),

wherein the polypeptide has a biological activity selected from the group consisting of: stimulating animal cell growth and/or division, or a component of connective tissue, or collagen fibrillogenesis.

Preferably, the polypeptide is at least 99% identical to a).

In another aspect, the present invention provides a substantially purified polypeptide comprising a sequence selected from the group consisting of:

a) a sequence as shown in SEQ ID NO:24, and

b) a sequence which is at least 98% identical to a),

wherein the polypeptide has a biological activity selected from the group consisting of: stimulating animal cell growth and/or division, or a component of collagen.

Preferably, the polypeptide is at least 99% identical to a).

The present invention also provides the deer ortholog of human transthyretin (SEQ ID NO:27) which comprises the sequences FVEGL/IYQ/KVEL/IDTK (SEQ ID NO: 41) and EGL/IYQ/KV (SEQ ID NO: 42).

In another aspect, the present invention provides a fusion protein comprising a polypeptide according to the present invention.

In a preferred embodiment of this aspect, the at least one other polypeptide is selected from the group consisting of: a polypeptide that enhances the stability of the polypeptide of the present invention, and a polypeptide that assists in the purification of the fusion protein.

In a further aspect, the present invention provides an isolated polynucleotide encoding a polypeptide according of the present invention.

Preferably, the polynucleotide comprises a sequence according to any one of SEQ ID NO:28, 29, 31 to 33, or 35 to 38.

In yet another aspect, the present invention provides an isolated polynucleotide comprising a sequence provided as SEQ ID NO:30.

In another aspect, the present invention provides an isolated polynucleotide comprising a sequence provided as SEQ ID NO:34.

In a further aspect, the present invention provides an antisense polynucleotide which hybridizes under high stringency conditions to a polynucleotide of the present invention.

In a further aspect, the present invention provides a vector comprising the polynucleotide according to the present invention.

Preferably, the polynucleotide is operably linked to a promoter.

The vectors may be nonviral (synthetic) or viral, as well as plasmid, or phage vectors provided with an origin of replication, and preferably a promoter for the expression of the polynucleotide molecule and, optionally, a regulator of the promoter. The vector may contain one or more selectable markers, for example, an ampicillin resistance gene in the case of a bacterial plasmid or a neomycin resistance gene for an animal expression vector. Other selectable markers may be used in accordance with the application at hand. The vector may be used in vitro, for example, for the production of RNA or used to transfect or transform a host cell.

In another aspect, the present invention provides a host cell transfected or transformed with a vector according to the present invention.

Preferably, the host cell is an animal cell. More preferably, the host cell is a mammalian cell.

In a further aspect, the invention provides a method of identifying and/or characterising the developmental position of mesenchymal cells, particularly during embryogenesis, the method comprising exposing a test sample including mesenchymal cell mRNA to a suitably-labelled nucleic acid probe with specifically hybridizes to a polynucleotide of the present invention and detecting hybridisation of said probe to said mRNA. Preferably, the test sample is a suitably prepared histological section.

In a further aspect, the present invention provides antibodies which specifically bind to a polypeptide of the present invention, as well as the use of the antibodies to block the ability of the polypeptide to stimulate cell growth and/or division.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In order that the present invention may be more clearly understood, preferred forms will-be described with reference to the following examples and drawings.

### BRIEF DESCRIPTION OF FIGURES

FIG. 1 shows a diagrammatic representation of endochondral bone formation in the foetus. In the early stages of embryogenesis, mesenchymal cells in the limb bud condense (A-C) to form a cartilaginous anlage (D). In the diaphysis of the anlage, chondrocytes hypertrophy and a boundary is formed between them and the surrounding undifferentiated stacked cells (E). Blood vessels invade the nondifferentiated cellular region of the anlage (F). A primitive marrow cavity is formed and the remaining cartilage establishes the epiphyseal growth plates (G). Secondary ossification centres arise concomitantly with vascularisation of the epiphysis allowing longitudinal growth (G) and (H) (Adapted from Cancedda et al. 1995).

FIG. 2 shows histochemical and immunohistochemical staining of proteoglycans and type II collagen in sections of 12-week-old human foetal distal phalanges to demonstrate their respective distribution in the tissues as well as the morphology of the endochondral ossification process. A(×16), D(×50), G(×100)=Masson Trichrome staining of collagens of dermis, connective tissue, blood vessels and blood cells of the foetal joint B(×16), E(×50), H(×100)=Toluidine Blue staining showing proteoglycan distribution in the epiphyseal hyaline cartilage. C(×16), F(×50), I(×100)=type II collagen immunostaining of hyaline cartilage complementary to proteoglycan distribution (Toluidine Blue). Note the invasion by blood vessels and resorption of cartilage matrix corresponding to early endochondral ossification of the central metaphysial shaft.

FIG. 3 is a diagrammatic representation of the cartilaginous (non-ossified) tip of deer antler showing the three main cellular regions designated as A, B and C corresponding to the PC (prechondrocyte), MC (mature chondrocyte) and HC (hypertrophic chondrocyte) phenotypes respectively. Panel A shows the tissue sampled only included the central cartilage core thereby excluding fibrous periosteum and regions considered to have undergone intramembranous ossification. Panel B shows cells from each of these three regions (A, B, C) were processed separately for cell culture studies and their total RNA extracted; also whole cartilaginous tip sections were used for histological, immunohistochemical, and in situ hybridisation studies, as well as total RNA was extracted from the whole cartilaginous tip.

FIG. 4 shows histochemical and immunohistochemical staining of cartilage sections n from region B (mature chondrocytes) of deer antler cartilage. Panel A(×50), B(×100)=Note Toluidine Blue staining of proteoglycan in cartilage matrix between vascular channels (unstained). Panels C(×50) and D(×100) show immunostaining for type II collagen of region B cartilage which is seen to be complementary to proteoglycan staining with Toluidine Blue (Panels A and B).

FIG. 5

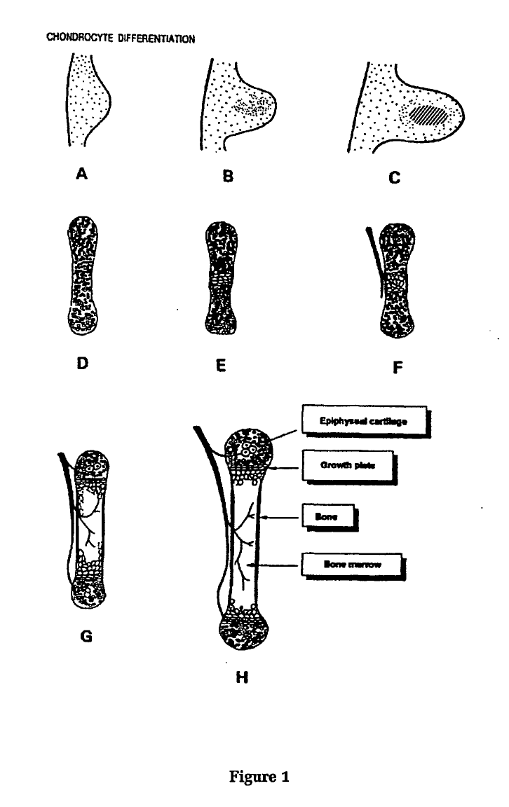

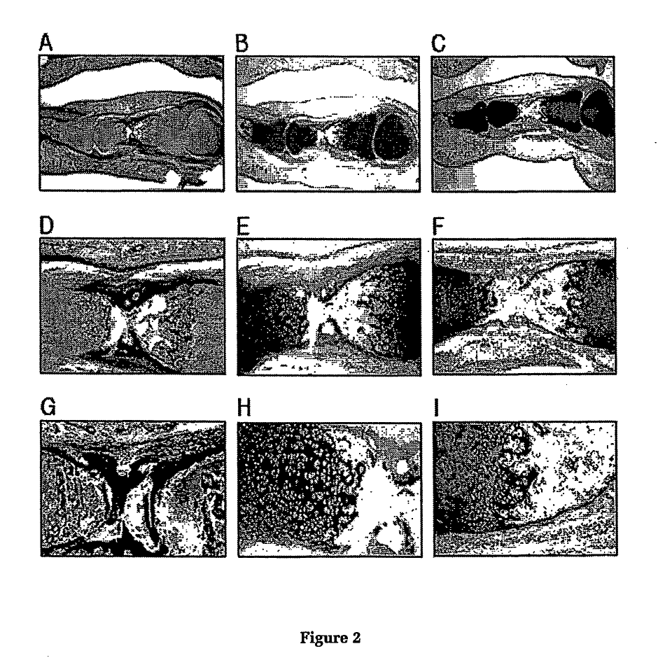

(i) DACC-2. Length: 1.426 kb. Underline indicates 93% homology of a 1.404 kb overlap with Human alpha1 type II collagen cDNA.

(ii) DACC-3. Length: 0.957 kb. Underline indicates 89% homology of a 0.876 kb overlap with Human ribosomal protein S2 (RPS2) cDNA.

(iii) DACC-4. Length: 0.532 kb. Underline indicates 92% homology of a 0.486 kb overlap with Human ribosomal protein L23a (RPL23A) cDNA.

(iv) DACC-5. Length: 1.224 kb. Underline indicates 89% homology of a 1.189 kb overlap with Human non-histone chromosomal protein (HMG-14) cDNA.

(v) DACC-6. Length: 1.163 kb. Underline indicates 91% homology of a 1.145 kb overlap with Human mRNA for KIAA1075 protein (tensin2) cDNA.

(vi) DACC-7. Length: 1.506 kb. Underline indicates 70% homology of a 1.506 kb (entire length) overlap with Human mRNA similar to RIKEN cDNA 0610011N22 gene (LOC133957, Genbank BC015349).

(vii) DACC-8. Length: 1.088 kb. Underline indicates 83% homology of a 1.086 kb overlap with Human SPARC/osteonectin cDNA.

(viiia) DACC-9 (5′end). Length: 0.410 kb. Underline indicates 89% homology of a 0.359 kb overlap with Human mRNA similar to HEAT-SHOCK 20 KDA LIKE-PROTEIN P20 (LOC126393, Genbank AK056951) and Human sequence 109 from Patent WO9954460 (Genbank AX013767) cDNA.

(viiib) DACC-9 (3′end). Length: 0.588 kb. Underline indicates 79% homology of a 0.584 kb overlap with Human mRNA similar to HEAT-SHOCK 20 KDA LIKE-PROTEIN P20 (LOC126393, Genbank AK056951) and Human sequence 109 from Patent WO9954460 (Genbank AX013767) cDNA.

(ix) DACC-10. Length: 1.625 kb. Underline indicates 90% homology of a 1.578 kb overlap with Human procollagen alpha2(V) cDNA.

(x) DACC-11. Length: 1.508 kb. Underline indicates 95% homology of a 1;508 kb overlap with Human prepro-alpha1(I) collagen cDNA.

FIG. 6 shows the incorporation of ³H-thymidine (counts per minute/microgram DNA) into DNA synthesised by vector alone (mock) and vector with DACC-7 transfected (DACC-7) lapine chondrocytes grown in pellet culture as described previously (Goomer et al. 2000). Note the higher incorporation of radioactivity into synthesised DNA of DACC-7 transfected cells.

FIG. 7 shows in situ hybridisation for DACC-7 mRNA on sections of 12-week-old human foetal knee joints showing expression of this gene product in epiphyseal hyaline cartilage but low expression in the meniscal cells. A(×16), C(×50), D(×100) DACC-7 mRNA, B(×16)=negative control.

FIG. 8 shows in situ hybridisation for type II collagen mRNA of sections of 12-week-old human foetal knee joints. A(×16), B(buffer only control), C(×50), D(×100). Note expression of type II collagen mRNA in both hyaline epiphyseal cartilage as well as the fibrocartilaginous meniscus (contrast with immunostaining where no type II collagen protein in meniscus was observed).

FIG. 9 shows in situ hybridisation for DACC-7 mRNA in sections of 12 and 14-week-old human foetal knee joint epiphyseal cartilage showing decreased expression of message in the 14-week-old relative to 12-week-old specimen. A(×200), C(×400)=12-week-old joint B(×200), D(×400)=14-week-old joint.

FIG. 10 shows a photomicrograph of sagittal histological section of the anterior region of a 12-week-old human foetal spinal column. Panel A: Toluidine Blue stained section showing disc and adjacent cartilaginous vertebral bodies, the notochordal cell cluster of the nucleus pulposus (NP) and the alignment of fibrocytes of the annulus fibrosis (AF) (×100). Panel B: Toluidine Blue stained section of disc and adjacent cartilaginous vertebral bodies showing NP and AF at higher magnification (×200). Panel C: Toluidine Blue stained section showing demarcation of cells in the cartilage anlage of the vertebral body and the adjacent fibrous AF(×400). Panel D: Higher power photomicrograph of the NP showing the notochordal cells and cells of the inner AF which will develop into the transitional zone (×400). Panel E: In situ hybridisation for DACC-7 expression by cells of the cartilage anlage and the transition to the AF using an antisense probe. Note the stronger shining of chondrocytes than fibrocytes (×400). Panel F: In situ hybridisation for DACC-7 expression by cells of the NP using an antisense probe. Note the strong staining of notochordal cells (×400).

FIG. 11 shows a photomicrograph of sagittal histological section of the anterior region of a 12-week-old human foetal spinal column. Panel A: In situ hybridisation for type II collagen expression by disc cells and the chondrocytes of the cartilage anlage of the vertebral body using a sense probe (×50). Panel B: In situ hybridisation for type II collagen expression by disc cells and the chondrocytes of the cartilage anlage of the vertebral body using a antisense probe showing expression in disc cells and cells of the adjacent cartilaginous vertebral bodies (×50). Panel C: In situ hybridisation for type II collagen expression by disc cells and the chondrocytes of the cartilage anlage of the vertebral body using a sense probe (×400). Panel D: In situ hybridisation for type II collagen expression by disc cells and the chondrocytes of the cartilage anlage of the vertebral body using an antisense probe showing demarcation of cells in the cartilage anlage of the vertebral body and the adjacent fibrous AF(×400). Panel E: In situ hybridisation for DACC-7 expression by the notochordal cell cluster of the nucleus pulposus (NP) using a sense probe (×400). Panel F: In situ hybridisation for DACC-7 expression by cells of the NP using an anti-sense probe. Note the strong staining of notochordal cells (×400).

FIG. 12 shows a photomicrograph of coronal histological sections of 14-week-old human foetal finger joint showing articulating surfaces and epiphyseal cartilage. Panel A: Toluidine Blue stained section showing proteoglycan distribution in the extracellular matrix of all cartilages and hypertrophic chondrocytes at the edge of the metaphysis (×50). Panel B: Toluidine Blue stained section showing proteoglycan distribution in cartilages of the articulating surfaces and epiphysis (×100). Panel C: In situ hybridisation for type II collagen expression by chondrocytes in serial sections of Panel B using a sense probe (×100). Panel D: In situ hybridisation for type II collagen expression by chondrocytes in serial sections of Panel B using an antisense probe (×100). Panel E: In situ hybridisation for DACC-7 expression by chondrocytes in serial sections of Panel B using a sense probe (×100). Panel F: In situ hybridisation for DACC-7 expression by chondrocytes in serial sections of Panel B using an antisense probe (×100).

FIG. 13 shows a photomicrograph of sagittal histological section of fragments of degenerate tibial plateau articular cartilage from a human OA joint. Panel A: Toluidine Blue stained section showing distribution of proteoglycans (×200). Panel B: Toluidine Blue stained section showing distribution of proteoglycans (×400). Panel C: In situ hybridisation of the OA cartilage cells for expression of type II collagen using a sense probe (×200). Panel D: In situ hybridisation of the OA cartilage cells for expression of type II collagen using an antisense probe (×200). Panel E: In situ hybridisation for DACC-7 expression by chondrocytes in OA cartilage using a sense probe (×200). Panel F: In situ hybridisation for DACC-7 expression by chondrocytes in OA cartilage using an antisense probe (×200). Panel G: In situ hybridisation for DACC-7 expression by chondrocytes in OA cartilage using a sense probe (×400). Panel H: In situ hybridisation for DACC-7 expression by chondrocytes in OA cartilage using an antisense probe (×400).

FIG. 14 shows a photomicrograph of horizontal histological sections of region B of fallow deer antler showing mature and hypertrophic chondrocytes assembled in a cartilaginous matrix surrounding the endothelium of vascular channels. Panel A: Toluidine Blue stained section (×200). Panel B: Toluidine Blue stained section (×400). Panel C: In situ hybridisation for type II collagen expression by antler chondrocytes using the sense probe (×400). Panel D: In situ hybridisation for type II collagen by antler chondrocytes using an antisense probe (×400). Panel E: In situ hybridisation for DACC-7 expression by antler chondrocytes using a sense probe (×200). Panel F: In situ hybridisation for DACC-7 expression by antler chondrocytes using an antisense probe (×200). Panel G: In situ hybridisation for DACC-7 expression by antler chondrocytes using a sense probe (×400). Panel H: In situ hybridisation for DACC-7 expression by antler chondrocytes using an antisense probe (×400).

FIG. 15 shows the predicted amino acid sequence, size and pI for DACC-7. The amino acid usage, identity and similarity with human (LOC133957) and mouse (RIKEN 0610011N22) homologs of DACC-7 are also shown.

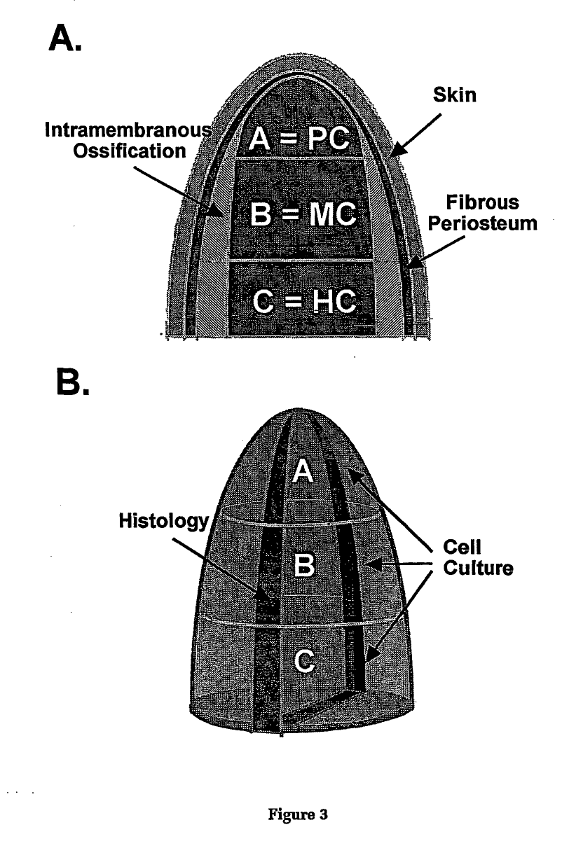

FIG. 16 shows the kinetics of stimulation of ³⁵S-PG synthesis in alginate beads of DAC cells from the three antler zones A, B, C. shown in FIG. 3. *B>A=C(p<0.05).

FIG. 17 shows the kinetics of DNA synthesis (as ³H-thymidine incorporation) by DAC cells from zones A, B, C cultured in alginate beads. *B>A=C(p<0.05).

FIG. 18 shows the kinetics of DNA synthesis by ovine articular chondrocytes cultured in the presence of bovine serum albumin (BSA), 10% foetal bovine serum (FBS) or conditioned media from alginate bead cultures of DAC cells from zones A or B from two different animals (2, 3).

FIG. 19 shows the kinetics of stimulation of ³⁵S-PG synthesis by ovine femoral condylar chondrocytes incubated for 24 h with various amounts of conditioned media (CM) from alginate bead cultures of DAC cells from zones A (□), B ( [symbol] ), C (▪). *A=B<C(p<0.05). #=relative to foetal bovine serum (FBS) alone.

FIG. 20 shows the kinetics of stimulation of ³⁵S-PG synthesis by ovine tibial plateau chondrocytes incubated for 24 h with various amounts of conditioned media (CM) from alginate bead cultures of DAC cells from zones A (□), B ( [symbol] ), C(▪). *A=B>C(p<0.05).

FIG. 21 shows the kinetics of DNA synthesis (³H-thymidine incorporation) by ovine chondrocytes incubated for 24 h with various amounts conditioned media (CM from alginate bead cultures of DAC cells from zones A (□), B ( [symbol] ), C (▪). *A=B>C(p<0.05). #=relative to foetal bovine serum (FBS) (p<0.05).

FIG. 22 shows the mitochondrial activity in ovine chondrocytes [determined using the MTT assay] after 24 h incubation with various concentrations of conditioned media (CM) from alginate bead cultures of DAC cells from zones A (□), B ( [symbol] ), C (▪). *B=C(p<0.05). #=relative to FBS (P<0.05).

FIG. 23 shows the kinetics of stimulation of ³⁵S-PG synthesis by ovine condylar chondrocytes incubated with conditioned media (CM) collected for up to 7 days from monolayer cultures of DAC cells (all zone) from different animals (F4, F5, R6.1, R6.2). (▪) 1 day, (□) 3 day ( [symbol] ) 5 day, ( [symbol] ) 7 day. #=relative to FBS alone (p<0.05).

FIG. 24 shows the kinetics of stimulation of ³⁵S-PG synthesis by lapine cartilage explants incubated with conditioned media (CM) collected for up 7 days from monolayer cultures of DAC cells from different animals (F4, F5, R6.1, R6.2). (▪) 1 day, (□) 3 day, ( [symbol] ) 5 days ( [symbol] ) 7 day. #=relative to FBS (p<0.05).

FIG. 25 shows the kinetics of DNA synthesis (³H-thymidine incorporation) by confluent murine 3T3 fibroblasts incubated with conditioned media (CM) from alginate bead cultures from different zones (A), (B), (C) from DAC or monolayer cultures (F4, F5, R6.1, R6.2). #=relative to FBS alone (p<0.05).

FIG. 26 shows a two-dimensional pH 5-8 gradient gel electrophoretogram of concentrated conditioned media from alginate cultures of deer antler chondrocytes which was collected over the first 24 h of culture. The spots circled in red were not present in 7 d cultures of the same cells which corresponded to loss of stimulatory activity of the culture media. Proteins 1 and 2 within these red circles were submitted for Q-TOF MS/MS mass spectrometry. Both proteins were identified as transthyretin on the basis of their partial amino acid sequences.

FIG. 27 shows restriction enzymes chosen for construction of a full length DACC-7 cDNA. The restriction enzymes used were EcoRI, SacI and KpnI. These enzymes were chosen on the basis of location in overlapping regions and order of restriction enzyme sites within the multiple cloning region of the plasmid.

Key to the Sequence Listing:

SEQ ID NO:1—Deer polypeptide sequence encoded by DACC-7.

SEQ ID NO:2—Human polypeptide orthologous to SEQ ID NO:1 (Accession No. XP_{—}059677)

SEQ ID NO:3—Mouse polypeptide orthologous to SEQ ID NO:1 (Accession No. NP_{—}077163).

SEQ ID NO:4—Deer polypeptide sequence encoded by DACC-2.

SEQ ID NO:5—Human polypeptide orthologous to SEQ ID NO:4 (Accession No. P02458).

SEQ ID NO:6—Mouse polypeptide orthologous to SEQ ID NO:4 (Accession No. B41182).

SEQ ID NO:7—Deer polypeptide sequence encoded by DACC-3.

SEQ ID NO:8—Human polypeptide orthologous to SEQ ID NO:7 (Accession No. P15880).

SEQ ID NO:9—Mouse polypeptide orthologous to SEQ ID NO:7 (Accession No. P25444).

SEQ ID NO:10—Deer polypeptide sequence encoded by DACC-4.

SEQ ID NO:11—Human polypeptide orthologous to SEQ ID NO:10 (Accession No. NP_{—}000975).

SEQ ID NO:12—Rat polypeptide orthologous to SEQ ID NO:10 (Accession No. CAA46336).

SEQ ID NO:13—Deer polypeptide sequence encoded by DACC-5.

SEQ ID NO:14—Human polypeptide orthologous to SEQ ID NO:13 (Accession No. XP_{—}049753).

SEQ ID NO:15—Deer polypeptide sequence encoded by DACC-6.

SEQ ID NO:16—Human polypeptide orthologous to SEQ ID NO:15 (Accession No. XP_{—}029631).

SEQ ID NO:17—Human polypeptide orthologous to protein encoded by full length cDNA comprising SEQ ID NO:34 (human osteonectin) (Accession No. P09486).

SEQ ID NO:18—Deer polypeptide sequence encoded by DACC-9.

SEQ ID NO:19—Human polypeptide orthologous to SEQ ID NO:18 (Accession No. XP_{—}059039).

SEQ ID NO:20—Rat polypeptide orthologous to SEQ ID NO:18 (Accession No. P97541).

SEQ ID NO:21—Deer polypeptide sequence encoded by DACC-10.

SEQ ID NO:22—Human polypeptide orthologous to SEQ ID NO:21 (Accession No. NP_{—}000384).

SEQ ID NO:23—Mouse polypeptide orthologous to SEQ ID NO:21 (Accession No. NP_{—}031763).

SEQ ID NO:24—Deer polypeptide sequence encoded by DACC-11.

SEQ ID NO:25—Human polypeptide orthologous to SEQ ID NO:24 (Accession No. AAB94054).

SEQ ID NO: 26—Mouse polypeptide orthologous to SEQ ID NO:24 (Accession No. P11087).

SEQ ID NO:27—Human transthyretin (Accession No. P02766).

SEQ ID NO:28—Deer cDNA sequence of clone DACC-2.

SEQ ID NO:29—Deer cDNA sequence of clone DACC-3.

SEQ ID NO:30—Deer cDNA sequence of clone DACC-4.

SEQ ID NO:31—Deer cDNA sequence of clone DACC-5.

SEQ ID NO:32—Deer cDNA sequence of clone DACC-6.

SEQ ID NO:33—Deer cDNA sequence of clone DACC-7.

SEQ ID NO:34—Deer cDNA sequence of clone DACC-8.

SEQ ID NO:35—Deer cDNA sequence of 5′ end of clone DACC-9.

SEQ ID NO:36—Deer cDNA sequence of 3′ end of clone DACC-9.

SEQ ID NO:37—Deer cDNA sequence of clone DACC-10.

SEQ ID NO:38—Deer cDNA sequence of clone DACC-11.

SEQ ID NO:39—Oligonucleotide primer.

SEQ ID NO:40—Oligonucleotide primer.

SEQ ID NO:41—N-terminal sequence of deer transthyretin protein fragment.

SEQ ID NO:42—N-terminal sequence of deer transthyretin protein fragment.

### DETAILED DESCRIPTION OF THE INVENTION

General Molecular Biology

Unless otherwise indicated, the recombinant DNA techniques utilised in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (Editors), Current Protocols in Molecular Biology, Greene Pub. Associates and Wiley-Interscience (1988, including all updates until present) and are incorporated herein by reference.

Polypeptides

By “substantially purified” we mean a polypeptide that has been separated from the lipids, nucleic acids, other polypeptides, and other contaminating molecules with which it is associated in its native state.

The % identity of a polypeptide is determined by GAP (Needleman and Wunsch, 1970) analysis (GCG program) with a gap creation penalty=5, and a gap extension penalty=0.3. The query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. Even more preferably, the query sequence is at least 100 amino acids in length and the GAP analysis aligns the two sequences over a region of at least 100 amino acids. More preferably, the query sequence is at least 250 amino acids in length and the GAP analysis aligns the two sequences over a region of at least 250 amino acids. Even more preferably, the query sequence is at least 500 amino acids in length and the GAP analysis aligns the two sequences over a region of at least 500 amino acids.

As used herein a “biologically active fragment” of a polypeptide used in the methods of the present invention is a portion of the polypeptide which maintains the ability to stimulate animal cell growth and/or division.

Polypeptides useful for the methods of the present invention can either be naturally occurring or mutants and/or fragments thereof.

Amino acid sequence mutants can be prepared by introducing appropriate nucleotide changes into DNA, or by in vitro synthesis of the desired polypeptide. Such mutants include, for example, deletions, insertions or substitutions of residues within the amino acid sequence. A combination of deletion, insertion and substitution can be made to arrive at the final construct, provided that the final protein product possesses the desired characteristics.

In designing amino acid sequence mutants, the location of the mutation site and the nature of the mutation will depend on characteristiC(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 30 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as the active and/or binding sitE(s). Other sites of interest are those in which particular residues obtained from various species are identical; These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

TABLE 1

| Original | Exemplary |
|---|---|
| Residue | Substitutions |
| Ala (A) | val; Leu; ile |
| Arg (R) | lys |
| Asn (N) | gln; his; |
| Asp (D) | glu |
| Cys (C) | ser |
| Gln (Q) | asn; his |
| Glu (E) | asp |
| Gly (G) | pro |
| His (H) | asn; gln |
| Ile (I) | leu; val; ala; norleucine |
| Leu (L) | norleucine, ile; val; |
| | met; ala; phe |
| Lys (K) | arg |
| Met (M) | leu; phe; |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe |
| | ala; norleucine |

Furthermore, if desired, unnatural amino acids or chemical amino acid analogues can be introduced as a substitution or addition into the polypeptide. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are polypeptides which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide.

Polypeptides can be produced in a variety of ways, including production and recovery of natural proteins, production and recovery of recombinant proteins, and chemical synthesis of the proteins. In one embodiment, an isolated polypeptide of the present invention is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit protein production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Polynucleotides

By “isolated polynucleotide” we mean a polynucleotide separated from the polynucleotide sequences with which it is associated or linked in its native state. Furthermore, the term “polynucleotide” is used interchangeably herein with the term “nucleic acid molecule”.

The % identity of a polynucleotide is determined by GAP (Needleman and Wunsch, 1970) analysis (GCG program) with a gap creation penalty=5, and a gap extension penalty=0.3. The query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides.

As used herein, high stringency conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 g/ml), 0.1% SDS and 10% dextran sulfate at 42° C. in 0.2×SSC and 0.1% SDS.

Polynucleotides may possess one or more mutations which are deletions, insertions, or substitutions of nucleotide residues. Mutants can be either naturally occurring (that is to say, isolated from a natural source) or synthetic (for example, by performing site-directed mutagenesis on the nucleic acid). It is thus apparent that polynucleotides can be either naturally occurring or recombinant.

Oligonucleotides of the present invention can be RNA, DNA, or derivatives of either. The minimum size of such oligonucleotides is the size required for the formation of a stable hybrid between an oligonucleotide and a complementary sequence on a nucleic acid molecule of the present invention. The present invention includes oligonucleotides that can be used as, for example, probes to identify nucleic acid molecules, primers to produce nucleic acid molecules or used to regulate the production of polypeptides as disclosed herein (e.g., as antisense-, triplex formation-, ribozyme- and/or RNA drug-based reagents). Oligonucleotide used as a probe are typically conjugated with a label such as a radioisotope, an enzyme, biotin, a fluorescent molecule or a chemiluminescent molecule.

Catalytic Nucleic Acids

The term catalytic nucleic acid refers to a DNA molecule or DNA-containing molecule (also known in the art as a “deoxyribozyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T and U, as well as derivatives thereof. Derivatives of these bases are well known in the art

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this invention are the hammerhead ribozyme (Haseloff and Gerlach 1988, Perriman et al., 1992) and the hairpin ribozyme (Shippy et al., 1999).

The ribozymes of this invention and DNA encoding the ribozymes can be chemically synthesized using methods well known in the art The ribozymes can also be prepared from a DNA molecule (that upon transcription, yields an RNA molecule) operably linked to an RNA polymerase promoter, e.g., the promoter for T7 RNA polymerase or SP6 RNA polymerase. Accordingly, also provided by this invention is a nucleic acid molecule, i.e., DNA or cDNA, coding for the ribozymes of this invention. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase. Alternatively, the ribozyme can be modified to the phosphothio analog for use in liposome delivery systems. This modification also renders the ribozyme resistant to endonuclease activity.

dsRNA

dsRNA is particularly useful for specifically inhibiting the production of a particular protein. Although not wishing to be limited by theory, Dougherty and Parks (1995) have provided a model for the mechanism by which dsRNA can be used to reduce protein production. This model has recently been modified and expanded by Waterhouse et al. (1998). This technology relies on the presence of dsRNA molecules that contain a sequence that is essentially identical to the mRNA of the gene of interest, in this case an mRNA encoding a polypeptide useful in the methods of the present invention. Conveniently, the dsRNA can be produced in a single open reading frame in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present invention is well within the capacity of a person skilled in the art, particularly considering Dougherty and Parks (1995), Waterhouse et al. (1998), WO 0.99/32619, WO 99/53050, WO 99/49029, and WO 01/34815.

Recombinant Vectors

One embodiment of the present invention includes a recombinant vector, which includes at least one isolated nucleic acid molecule encoding a polypeptide useful for the methods of the present invention, inserted into any vector capable of delivering the nucleic acid molecule into a host cell. Such a vector contains heterologous nucleic acid sequences, that is nucleic acid sequences that are not naturally found adjacent to nucleic acid molecules encoding a polypeptide useful for the methods of the present invention and that preferably are derived from a species other than the species from which the nucleic acid moleculE(s) are derived. The vector can be either RNA or DNA, either prokaryotic or eukaryotic, and typically is a virus or a plasmid.

One type of recombinant vector comprises a nucleic acid molecule encoding a polypeptide useful for the methods of the present invention operably linked to an expression vector. The phrase operably linked refers to insertion of a nucleic acid molecule into an expression vector in a manner such that the molecule is able to be expressed when transformed into a host cell. As used herein, an expression vector is a DNA or RNA vector that is capable of transforming a host cell and of effecting expression of a specified nucleic acid molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, endoparasite, arthropod, other animal, and plant cells. Preferred expression vectors useful for the methods of the present invention can direct gene expression in bacterial; yeast, arthropod and mammalian cells and more preferably in the cell types disclosed herein. Most preferably, vectors useful for the methods of the present invention can direct gene expression in mammalian cells.

Expression vectors of the present invention contain regulatory sequences such as transcription control sequences, translation control sequences, origins of replication, and other regulatory sequences that are compatible with the recombinant cell and that control the expression of nucleic acid molecules useful for the methods of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, arthropod and mammalian cells, such as, but not limited to, tac, lac, trp, trc, oxy-pro, omp/lpp, rrnB, bacteriophage lambda, bacteriophage T7, T71ac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells. Additional suitable transcription control sequences include tissue-specific promoters and enhancers as well as lymphokine-inducible promoters (e.g., promoters inducible by interferons or interleukins). Transcription control sequences of the present invention are most preferably naturally occurring transcription control sequences naturally associated with mammals.

Recombinant molecules of the present invention may also (a) contain secretory signals (i.e., signal segment nucleic acid sequences) to enable an expressed polypeptide useful for the methods of the present invention to be secreted from the cell that produces the polypeptide and/or (b) contain fusion sequences which lead to the expression of fusion proteins. Examples of suitable signal segments include any signal segment capable of directing the secretion of the fusion protein. Preferred signal segments include, but are not limited to, tissue plasminogen activator (t-PA), interferon, interleukin, growth hormone, histocompatibility and viral envelope glycoprotein signal segments, as well as natural signal sequences. Suitable fusion segments encoded by fusion segment nucleic acids are disclosed herein. In addition, a nucleic acid molecule useful for the methods of the present invention can be joined to a fusion segment that directs the encoded protein to the proteosome, such as a ubiquitin fusion segment. Recombinant molecules may also include intervening and/or untranslated sequences surrounding and/or within the nucleic acid sequences.

Host Cells

Another embodiment of the present invention includes a recombinant cell comprising a host cell transformed with one or more recombinant molecules useful for the methods of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide of the present invention. Host cells can be either untransformed cells or cells that are already transformed with at least one nucleic acid molecule (e.g., nucleic acid molecules encoding one or more proteins of the present invention). Host cells useful for the methods of the present invention either can be endogenously (i.e., naturally) capable of producing the expressed protein or can be capable of producing such proteins after being transformed with an expression vector as disclosed herein. Host cells of the present invention can be any cell capable of producing at least one protein useful for the methods of the present invention, and include bacterial, fungal (including yeast), parasite, arthropod, plant and animal cells. Most preferably, the host cell is a mammalian cell.

Suitable prokaryotes include, but are not limited to, eubacteria, such as Gram-negative or Gram-positive organisms, for example, Escherichia coli, Bacilli such as B. subtilis or B. thuringiensis, Pseudomonas species such as P. aeruginosa, Salmonella typhimurium or Serratia marcescens.

Eukaryotic microbes such as filamentous fungi or yeast are suitable hosts for expressing the protein(s) of the present invention. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as e.g. K. lactis; filamentous fungi such as, e.g. Neurospora, or Penicillium; and Aspergillus hosts such as A. nidulans and A. niger.

Suitable higher eukaryotic host cells can be cultured vertebrate, invertebrate or plant cells. Insect host cells from species such as Spodoptera frugiperda, Aedes aegypti, Aedes albopictus, Drosophila melanogaster; and Bombyx mori can be used. Plant cell cultures of cotton, corn, potato, soybean, tomato, and tobacco can be utilised as hosts. Typically, plant cells are transfected by incubation with certain strains for the bacterium Agrobacterium tumefaciens.

Propagation of animal cells in culture (tissue culture) has become a routine procedure in recent years. Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK ATCC CCL 10); Chinese hamster ovary cells/-DHFR(CHO); mouse sertoli cells, monkey kidney cells (CV1 ATCC CCL 70); human cervical carcinoma cells (HELA, ATCC CCL 2); canine kidney cells (MDCK ATCC CCL 34), and a human hepatoma cell line (Hep G2). Preferred host cells are human embryonic kidney 293 and Chinese hamster ovary cells.

The host cell may also be selected from mammalian foetal cells, particularly human foetal cells. Especially preferred are chondrocytes including human chondrocytes, or other mesenchymal cells including human mesenchymal stem cells. Such transformed or transfected host cells may be used for, for example, xenotransplantation (i.e. where the host cell is of other mammalian origin) or autotransplantation (i.e. where the host cell originates from the recipient) to a human subject

Host cells are transfected and preferably transformed with expression or cloning vectors of this invention and cultured in conventional nutrient media modified as appropriate for inducing promoters, selecting transformants, or amplifying the genes encoding the desired sequences. Transformation means introducing DNA into an organism so that the DNA is replicable, either as an extrachromosomal element or by chromosomal integration. Depending on the host cell used, transformation is done using standard techniques appropriate to such cells.

Recombinant DNA technologies can be used to improve the expression of transformed polynucleotide molecules by manipulating, for example, the number of copies of the polynucleotide molecules within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules useful for the methods of the present invention include, but are not limited to, operably linking polynucleotide molecules to high-copy number plasmids, integration of the polynucleotide molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules of the present invention to correspond to the codon usage of the host cell, and the deletion of sequences that destabilize transcripts. The activity of an expressed recombinant protein of the present invention maybe improved by fragmenting, modifying, or derivatizing polynucleotide molecules encoding such a protein.

Gene Therapy

The polynucleotides, polypeptides, agonists and antagonists that are polypeptides, may be employed in accordance with the present invention by expression of such polypeptides in treatment modalities often referred to as “gene therapy”. Thus, for example, cells from a patient may be engineered with a polynucleotide, such as a DNA or RNA, to encode a polypeptide ex vivo. The engineered cells can then be provided to a patient to be treated with the polypeptide. In this embodiment, cells may be engineered ex vivo, for example, by the use of a retroviral plasmid vector containing RNA encoding a polypeptide useful for the methods of the present invention can be used to transform stem cells or differentiated stem cells. Such methods are well-known in the art and their use in the present invention will be apparent from the teachings herein.

Further, cells may be engineered in vivo for expression of a polypeptide in vivo by procedures known in the art For example, a polynucleotide useful for a method of the present invention may be engineered for expression in a replication defective retroviral vector or adenoviral vector or other vector (e.g., poxvirus vectors). The expression construct may then be isolated. A packaging cell is transduced with a plasmid vector containing RNA encoding a polypeptide useful for a method of the present invention, such that the packaging cell now produces infectious viral particles containing the gene of interest These producer cells may be administered to a patient for engineering cells in vivo and expression of the polypeptide in vivo. These and other methods for administering a polypeptide of the present invention should be apparent to those skilled in the art from the teachings of the present invention.

Retroviruses from which the retroviral plasmid vectors hereinabove-mentioned may be derived include, but are not limited to, Moloney Murine Leukemia Virus, Spleen Necrosis Virus, Rous Sarcoma Virus, Harvey Sarcoma Virus, Avian Leukosis Virus, Gibbon Ape Leukemia Virus, Human Immunodeficiency Virus, Adenovirus, Myeloproliferative Sarcoma Virus, and Mammary Tumor Virus. In a preferred embodiment, the retroviral plasmid vector is derived from Moloney Murine Leukemia Virus.

Such vectors will include one or more promoters for expressing the polypeptide. Suitable promoters which may be employed include, but are not limited to, the retroviral LTR; the SV40 promoter; and the human cytomegalovirus (CMV) promoter. Cellular promoters such as eukaryotic cellular promoters including, but not limited to, the histone, RNA polymerase III, and β-actin promoters, can also be used. Additional viral promoters which may be employed include, but are not limited to, adenovirus promoters, thymidine kinase (TK) promoters, and B19 parvovirus promoters. The selection of a suitable promoter will be apparent to those skilled in the art from the teachings contained herein.

The nucleic acid sequence encoding the polypeptide useful for a method of the present invention will be placed under the control of a suitable promoter. Suitable promoters which may be employed include, but are not limited to, adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs (including the modified retroviral LTRs herein above described); the β-actin promoter; and human growth hormone promoters. The promoter may also be the native promoter which controls the gene encoding the polypeptide.

The retroviral plasmid vector is employed to transduce packaging cell lines to form producer cell lines. Examples of packaging cells which may be transfected include, but are not limited to, the PE501, PA317, Y-2, Y-AM, PA12, T19-14×, VT-19-17-H2, YCRE, YCRIP, GP+E-86, GP+envAm12, and DAN cell lines as described in Miller (1990). The vector may be transduced into the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line will generate infectious retroviral vector particles, which include the nucleic acid sequencE(s) encoding the polypeptides. Such retroviral vector particles may then be employed to transduce eukaryotic cells, either in vitro or in vivo. The transduced eukaryotic cells will express the nucleic acid sequencE(s) encoding the polypeptide. Eukaryotic cells which may be transduced include, but are not limited to, mesenchemymal cells, chondrocytes, embryonic stem cells, embryonic carcinoma cells, as well as hematopoietic stem cells, hepatocytes, fibroblasts, myoblasts, keratinocytes, endothelial cells, and bronchial epithelial cells.

Genetic therapies in accordance with the present invention may involve a transient (temporary) presence of the gene therapy polynucleotide in the patient or the permanent introduction of a polynucleotide into the patient.

Genetic therapies, like the direct administration of agents discussed above, in accordance with the present invention may be used alone or in conjunction with other therapeutic modalities.

Compositions and Administration

Compositions useful for a method of the present invention comprise an acceptable carrier. Typically, the carrier will also be considered as a “pharmaceutically acceptable carrier”, meaning that it is suitable to be administered to an mammal, preferably a human. Suitable carriers include isotonic saline solutions, for example phosphate-buffered saline.

The composition of the invention may be administered by direct injection. The composition may be formulated for, as examples, parenteral, intramuscular, intravenous, subcutaneous, intraocular, oral or transdermal administration. Typically, each protein (for example) may be administered at a dose of from 0.01 to 30 mg/kg body weight, preferably from 0.1 to 10 mg/kg, more preferably from 0.1 to 1 mg/kg body weight. The routes of administration and dosages described are intended only as a guide since a skilled practitioner will be able to determine readily the optimum route of administration and dosage for any particular, compound, animal and condition.

Polynucleotides/vectors encoding polypeptide components for use in affecting viral infections may be administered directly as a naked nucleic acid construct, preferably further comprising flanking sequences homologous to the host cell genome. When the polynucleotides/vectors are administered as a naked nucleic acid, the amount of nucleic acid administered may typically be in the range of from 1 μg to 10 mg, preferably from 100 μg to 1 mg. Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

One embodiment of the present invention is a controlled release formulation that is capable of slowly releasing a composition useful for a method of the present invention into an animal. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Preferred controlled release formulations are biodegradable (i.e., bioerodible).

Methods of Screening for Modulators of Polypeptide Activity

As used herein a “lead compound” is an agent identified by the methods of the present invention which is subject to trials with the goal of ultimately being formulated in, for example, a composition and sold as an agent for stimulating cell growth and/or division.

Known screening techniques can be used to identify agents which modulate the activity, or production of, a polypeptide of the present invention which stimulates cell growth and/or division. For instance, a candidate agents can be exposed to a cell in the presence or absence of the polypeptide, and the resulting effects on cell growth and/or division analysed, through standard techniques such as measuring cell numbers or DNA synthesis, to determine if the candidate agent directly effects the activity of the polypeptide,

Another method for screening for agonists/antagonists involves mixing the polypeptide with a binding partner (which is capable of binding to the polypeptide) and measuring their binding to each other in the presence or absence of a potential agonist/antagonist The polypeptide or the binding partner can be detectably labeled using known labels such as those selected from the group consisting of: radioisotopes, fluorophores and chromophores. This binding assay may be in the form of an ELISA plate assay. There are other binding formats known to those of skill in the art, including coprecipitation, centrifugation and surface plasmon resonance.

One potential antagonist is a small molecule which binds to the polypeptide. Examples of small molecules include, but are not limited to, small peptides, peptide-like molecules, plant secondary metabolites or synthetic organic chemicals.

As described herein, suitable antisense polynucleotide and dsRNA molecules can be designed based on the sequences of a polynucleotide encoding the polypeptide. Such antisense polynucleotide and dsRNA molecules can be used as agents for modulating cell growth and/or division when a cell has transformed with the antisense polynucleotide or dsRNA molecule.

Such antisense polynucleotides and dsRNA molecules can also be screened for use as an agent using the methods of the present invention. For instance, a polynucleotide encoding the polypeptide of interest can be expressed in a cell system, or a cell-free expression system, resulting in the production of the polypeptide. Candidate antisense polynucleotides and dsRNA molecules designed based on the can be incorporated into the system and the resulting affects on transcribed mRNA levels or polypeptide levels or activity, can readily be measured using techniques known in the art.

Suitable inhibitors of a polypeptide's ability to stimulate cell growth and/or division are compounds that interact directly with a protein's active site, thereby inhibiting activity.

Effective amounts and dosing regimens for the application of agents identified by the methods of the present invention can readily be determined using techniques known to those skilled in the art.

Phage Libraries for Candidate Agent Screening

Phage libraries can be constructed which when infected into host E. coli produce random peptide sequences of approximately 10 to 15 amino acids. Specifically, the phage library can be mixed in low dilutions with permissive E. coli in low melting point LB agar which is then-poured on top of LB agar plates. After incubating the plates at 37° C. for a period of time, small clear plaques in a lawn of E. coli will form which represents active phage growth and lysis of the E. coli. A representative of these phages can be absorbed to nylon filters by placing dry filters onto the agar plates. The filters can be marked for orientation, removed, and placed in washing solutions to block any remaining absorbent sites. The filters can then be placed in a solution containing, for example, a radioactively labeled polypeptide useful for the methods of the present invention (e.g., a polypeptide having an amino acid sequence comprising SEQ ID NO:1). After a specified incubation period, the filters can be thoroughly washed and developed for autoradiography. This allows plaques containing the phage that bind to the radioactive polypeptide to be detected. These phages can be further cloned and then retested for their ability to bind to the polypeptide as before. Once the phages have been purified, the binding sequence contained within the phage can be determined by standard DNA sequencing techniques. Once the DNA sequence is known, synthetic peptides can be generated which represents these sequences.

The effective peptidE(s) can be synthesized in large quantities for use in in vivo models and eventually as an agent for modulating cell growth and/or division. It should be emphasized that synthetic peptide production is relatively non-labor intensive, easily manufactured, quality controlled and thus, large quantities of the desired product can be produced rather cheaply.

Hybrid Screening Techniques

In yet another embodiment of the invention, the polypeptides can be used as “bait proteins” in a two-hybrid assay or three-hybrid assay (see, for example, U.S. Pat. No. 5,283,317 and WO94/10300), to identify other proteins, which bind to or interact with the polypeptide and are involved in modulating cell growth and/or division.

The two-hybrid system is based on the modular nature of most transcription factors, which consist of separable DNA-binding and activation domains. Briefly, the assay utilizes two different DNA constructs. In one construct, the gene that codes for the polypeptide of interest is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the polypeptide of interest

Protein-Structure Based Design of Candidate Agents

Crystals of a polypeptide useful for the methods of the present invention could be grown by a number of techniques including batch crystallation, vapour diffusion (either by sitting drop or hanging drop) and by microdialysis. Seeding of the crystals in some instances could be required to obtain X-ray quality crystals. Standard micro and/or macro seeding of crystals may therefore be used. Once a crystal is grown, X-ray diffraction data can be collected using standard techniques.

Once the three-dimensional structure of the polypeptide is determined, a potential antagonist or agonist can be examined through the use of computer modeling using a docking program such as GRAM, DOCK, or AUTODOCK (Dunbrack et al., 1997). This procedure can include computer fitting of potential ligands to the polypeptide to ascertain how well the shape and the chemical structure of the potential ligand will complement or interfere with the activity of the polypeptide. Computer programs can also be employed to estimate the attraction, repulsion, and steric hindrance of the ligand to the polypeptide. Generally the tighter the fit (e.g., the lower the steric hindrance, and/or the greater the attractive force) the more potent the potential agent will be since these properties are consistent with a tighter binding constant. Furthermore, the more specificity in the design of a potential agent the more likely that the agent will not interfere with other proteins. This will minimize potential side-effects due to unwanted interactions with other proteins.

Initially a potential compound could be obtained, for example, by screening a random peptide library produced by a recombinant bacteriophage as described above, or a chemical library. A compound selected in this manner could be then be systematically modified by computer modeling programs until one or more promising potential compounds are identified.

Such computer modeling allows the selection of a finite number of rational chemical modifications, as opposed to the countless number of essentially random chemical modifications that could be made, and of which any one might lead to a useful agent Each chemical modification requires additional chemical steps, which while being reasonable for the synthesis of a finite number of compounds, quickly becomes overwhelming if all possible modifications needed to be synthesized. Thus through the use of the three-dimensional structure and computer modeling, a large number of these compounds can be rapidly screened on the computer monitor screen, and a few likely candidates can be determined without the laborious synthesis of untold numbers of compounds.

The prospective agent can be placed into any standard binding assay to test its effect

Methods

General

Radionucleotides

Alpha linked radioactive phosphorus [α³²P] 2′-deoxycytidine 5′-triphosphate (dCTP), gamma linked [g³²P] 2′-deoxyadenosine 5′-triphosphate (dATP), [α³²P] 2′-deoxyuridine (dUTP) and [α³⁵S] dATP nucleotides were obtained from Dupont NEN® (Wilmington, Del., USA).

Restriction Enzymes

All restriction enzymes used were obtained from Roche (Roche Molecular Systems, Inc., NJ, USA).

Primers

All primers were commercially obtained from Bresatec Limited (SA, Australia) and were received in desiccated form. Pellets were resuspended in sterile water (Baxter, NSW) to a concentration of 1 mg/mL and stored at −70° C. Working solutions at 100 ng/mL were diluted from this stock concentration and stored at 4° C.

Polymerase Chain Reaction (PCR)

The reagents used for PCR were obtained from three main sources. For most PCR reactions, MgCl₂ solution of 25 mM, 10×Taq polymerase were obtained from Perkin-Elmer (Roche Molecular Systems, Inc., NJ, USA). Where greater sensitivity of PCR was needed an Advantages cDNA PCR kit (CLONTECH Laboratories, Inc., USA) or a PLATINUM® Taq DNA polymerase High Fidelity (Gibco BRL, Life Technologies) was used. A Perkin Elmer Cetus DNA thermal cycler machine was used and the number of cycles applied was dependent on the type of polymerase used and the nature of the reaction. The most common cycles used were 94° C. for 5 min; followed by 35 cycles of 94° C. for 1 min, 55° C. for 39 s, 72° C. for 1 min.

Agarose Gel Electrophoresis

All agarose gels were made using 1×TAE (40 mM Tris-acetate, 1 mM EDTA pH 8.0) for both the gel and running buffer. 1% agarose/TAE gels were made using agarose type I (Sigma Chemical Co., St Louis, Mo., USA). Loading buffer for all samples consisted of 0.25% bromophenol blue and 40% (w/v) sucrose in water. A concentration of 0.5 μg/mL of ethidium bromide was used for each gel.

Two types of horizontal gel apparatus were used. For 30 ml gels, a HORIZON® 58 Gel tank (BRL, Life Technologies Inc, Gaithersburg, Md., USA) was used. An Extra-wide Minigel system model D2 (Owl Scientific Plastics Inc, Cambridge, Mass., USA) was used for 70 mL gels in analysing larger numbers of samples. Samples were electrophoresed using a LKB, Bromma power pack 2197 (Uppsala, Sweden) or an EPS 600 power pack (Pharmacia Biotech, Sweden) at 80 to 120 V and a time range of 20 to 60 min.

Purification and Concentration of DNA

To purify and concentrate DNA after restriction digestion or from agarose gels, a QIAquick® Gel extraction Kit (QIAGEN Pty Ltd, Vic, Australia). DNA bands of interest on agarose gels were isolated in 1.5 mL microcentrifuge tubes. The gel slice was then incubated at 50° C. until complete melting of the agarose and processed as per kit instructions. For DNA to be purified from restriction enzymes, no incubation at 50° C. was required, instead was processed directly as per kit instructions. The purified DNA samples were stored at 4° C.

Preparation of Plasmid DNA

Minipreparation of Plasmid DNA

For small amounts of plasmid DNA, a Wizard® Plus Minipreps DNA Purification System (Promega Corp., NSW, Australia) was used. This system came as a kit, providing a reliable method for good quality plasmid DNA. Three microlitres of bacterial culture in LB with the appropriate supplements was inoculated from a colony or pure culture and incubated at 37° C. overnight with shaking. One point-five microlitres of overnight bacterial culture was placed in a 1.5 mL microfuge tube and spun in a microcentrifuge for 30 s, after which the supernatant was discarded. The cell pellet was then processed as per kit instructions. The DNA was eluted in 50 μL of 1×TE(1 M Tris/0.5 M EDTA, pH 8.0). The quality of the DNA was analysed by test digestion of 5 μL with appropriate restriction enzymes and running on an agarose gel.

Midipreparation of Plasmid DNA

For larger amounts of plasmid DNA and to prepare DNA for sequencing, a QIAGEN® Plasmid Midi kit was used. 25 mL of LB with appropriate supplements was inoculated with a pure bacterial colony and incubated with shaking at 37° C. overnight The overnight culture was transferred into a 250 mL centrifuge bottle and spun in a Beckmann® XL-90 (Beckmann Instruments, Inc., CA, USA) or Sorvall® RC 5C Plus (Dupont Australia Ltd., Sydney, Australia) ultracentrifuge at 8,000 rpm for 10 min to pellet the bacteria The bacterial pellet was then processed as per kit instructions. The DNA pellet was redissolved in 200 μL of 1×TE. Spectrophotometer readings (on a Beckmnann Du®-68 machine) were taken to determine the yield of plasmid DNA. DNA concentration was calculated using the formula: 1.0 unit of optical density at 260 nm is equivalent to 50 μg/mL dsDNA.

DNA Sequencing

High purity double stranded DNA template for sequencing was generated by the above procedure. This template was sent to SUPAMAC (Sydney University and Prince Alfred Macromolecular Analysis Centre, Sydney, Australia) or AGRF (Australian Genome Research Facility, Brisbane, Australia) where the template was sequenced by dye-terminator chemistry. With this method, 4 dye-labelled dideoxy nucleotides replace standard dideoxy nucleotides, incorporating into the DNA as the terminating base. Universal primers T7, SP6, T3, and −21M13 (Forward and Reverse) were used in the cycle sequencing reaction. The fluorescent signal for each base was tracked to produce an electropherogram file, displaying different bases of the sequence as peaks, where individual peaks were labelled with one of four different colours corresponding to the four bases (A, G, C, and T). This file of raw data was obtained for analysis. The sequence data was analysed using the Sequencher® program (version 3, Genes Codes Corp., Ann Arbor, Mich., USA).

RNA Techniques

All reagents were made using diethylpyrocarbonate (DEPC) treated water. Dedicated glassware and pipette tips were used, and gloves were worn at all times to minimise the risk of contamination by RNases.

RNA Probes

To create an RNA probe, it was necessary to clone the cDNA product into a suitable vector (such as pGEM T-Easy®) that contained RNA polymerase binding sites (such as SP6 and T7), allowing single stranded RNA to be manufactured. This was transformed into host bacteria (as described later) and plasmid was obtained by miniprep (as described previously).

Radioactively Labelled cRNA Probe Preparation

Two micrograms of linearised DNA template containing the insert cloned in a suitable vector was combined in a screw-topped tube with 4 μL of 5×transcription buffer (200 mM Tris HCl, pH7.5; 30 mM MgCl₂; 25 mM NaCl), 2 μL of 0.2 M dithiothreitol (DTT), 1 μL of rRNAsin® RNase inhibitor (Promega Corp., Madison, Wis., USA), 4 μL of ATP, CTP, GTP (Pharmacia LKB Biotechnology, Boronia, Australia) mixture was added. This mixture was vortexed and spun down. Then, 2.5 μL of α³²P-dUTP and 2.5 μL of appropriate RNA polymerase was added to the mixture and incubated at 37° C. for 1 h. The DNA template was degraded with 5 μL of mixture containing 200 units of DNaseI (GibcoBRL Life Technologies), 9 μL of DEPC water and 2.5 units of rRNAsin at 37° C. for 10 min.

The radiolabelled riboprobe was purified using an Elutip-D column as per manufacturer's instructions (Schleicher and Schell, Dassel, Germany). The radioactive product was eluted in 300 μL of high salt buffer (1 M NaCl; 0.01 M Tris, pH 8.0; 1 mM EDTA, pH 8.0). Two microlitres were removed from the 300 μL and used to measure the radioactivity of the probe on a [symbol] 0 counter (Tricarb™ Liquid Scintillation Analyser 1600TR, Packard Instruments Co., Canberra, Australia). Only those with a measured radioactivity of at least 50,000 cpm/μL were used for hybridisation. The rest was immediately frozen at −70° C. and was used within 24 h.

RNA Preparation

Cell and Tissue Specimen Preparation

Human foetal cartilage tissue was provided by Dr. Bernie Tuch (Prince of Wales Hospital, Sydney, Australia) and Dr. Sue Craig (Royal North Shore Hospital, Sydney, Australia). Their collection and use for this study was approved by the Royal North Shore Hospital Human Research Ethics Committee (HREC). Cartilage samples of adult deer antler (whole and regions), adult deer articular cartilage, 6 week old sheep growth plate cartilage, 6 week old sheep articular cartilage, 6 week old sheep sternal cartilage, adult human articular cartilage, 4 weeks to term foetal male deer epiphyseal cartilage, 4 weeks to term foetal male deer intervertebrate disc cartilage, 4 weeks to term foetal male deer rib cartilage, 4 weeks to term foetal male deer sternal cartilage, and 4 weeks to term foetal male deer calvaria cartilage (were provided by Mr Denis White of ADP Pharmaceutical Pty Limited, Goulburn, NSW, Australia) were taken for RNA analysis. These samples were either collected by snap freezing in liquid nitrogen (as with the whole deer antler, skin removed) or were enzymatically digested first to release cells, the chondrocytes collected by centrifugation then snap frozen in liquid nitrogen. The enzymatic digestion procedure was the preferred method for preparation of RNA as extraction directly from snap frozen tissue gave very low RNA yields.

A typical procedure was performed as follows: Immediately after sacrifice (or in the case of deer antler, after harvesting from the animal after administering local anaesthetic (Lignocaine) to front and back veins) specimens were transported to the laboratory in plastic bags maintained at 4° C. on ice. The specimens were thoroughly sprayed with 70% (v/v) ethanol and surrounding tissue (in particular, mesenchymal) was carefully removed under sterile conditions to obtain only target cartilage. The deer antler cartilage (DAC) regions were discernible by the pre-chondrocyte tissue observed as white, soft cartilage with no blood vessels; the mature chondrocyte tissue observed as soft cartilage with blood vessels; and the hypertrophic chondrocyte tissue observed as hard mineralised cartilage full of blood vessels. The outer rim of cartilage (intramembranous ossification) was discarded in each DAC region.

The cartilage was digested for 2h at 37° C. in 0.1% (w/v) pronase (Boehringer Mannheim Australia Pty Ltd, Castle Hill, NSW, Australia) in Hams F12 media (Trace Biosciences Pty Ltd, Castle Hill, NSW, Australia) supplemented with 10% (v/v) foetal bovine serum (Trace Biosciences), 76 mM NaHCO₃, 20 mM HEPES (Sigma Chemical Company, St Louis, Mich., USA) and 80 units per mL gentamycin (Delta West Pty Ltd, WA, Australia). This was then replaced with media containing 0.04% (w/v) collagenase (Sigma) for digestion overnight at 37° C. For DAC, the digestion procedure was 0.125% (w/v) trypsin (Sigma) in 1:1 DMEM (Sigma)/Hams F12 (DMEM:F12) media supplemented with 78 mM NaHCO₃, 20 mM HEPES, 80 units per mL gentamycin at 4° C. overnight, then 37° C. for 1 h. This was replaced with media containing 0.04% (w/v) collagenase and supplemented with 10% (v/v) foetal bovine serum at 37° C. for 3-4 h, vortexing for 10 sec every 30 min. Cells were collected through a sterile 70 μm Cell Strainer (Becton Dickinson, Franklin Lakes, N.J., USA) and pelleted for RNA extraction.

Extraction of Total RNA

Cell pellets (or in the case of whole deer antler, tissue samples) were removed from the −70° C. freezer and placed on dry ice. The tissue sample of whole deer antler was homogenised first in mortars filled with liquid nitrogen. The cell pellet (10×10⁶ cells) or 50 mg tissue sample was sonicated after adding 1 mL of TRI Reagents (Molecular Research Center, Inc., Cincinnati, Ohio, USA). TRI Reagent® was used as it has a higher recovery of undegraded mRNAs than other RNA extraction methods, which was essential for this analysis. The total RNA was then extracted from samples using the manufacture's protocol (TRI Reagent—RNA, DNA, and protein isolation reagent Manufacturer's protocol (1995), Molecular Research Center). The final dried total RNA pellet was resuspended into 50 μL of DEPC treated water and stored at −70° C.

Quantification of RNA

Spectrophotometer readings (on a Beckmann Du®-68 machine) were taken to determine the yield of RNA. RNA concentration was calculated using the formula: 1.0 unit of optical density at 260 nm is equivalent to 40 μg/mL RNA.

Northern Blot Analysis

Northern Blot Preparation

Total RNA samples (5 μg) were vacuum dried and resuspended into 15 μL of blue juice mix loading buffer, consisting of 20% (v/v) formaldehyde, 40% (v/v) deionised formamide, 1×MOPS (200 mM MOPS (Sigma), 50 mM Na acetate, 10 mM disodium EDTA, pH 7.0) and 12% (v/v) “blue juice” (50% (v/v) glycerol (Ajax Chemicals, Auburn, NSW, Australia), 1% (v/v) EDTA, 0.4% (v/v) bromophenol blue (International Biotechnologies Inc., New Haven, Conn., USA)). The samples were denatured at 65° C. for 3 minutes then were fractionated by electrophoresis at 110 V for 4-4.5 h on a 1% (w/v) agarose (2.2 M formaldehyde) gel. 0.24-9.5 kb RNA ladder (GibcoBRL Life Technologies, Gaithersburg, Md., USA) was also included. The gel was stained with ethidium bromide and transilluminated with ultraviolet light to visualise the 28S and 18 S rRNA. After photographing, the gel was rinsed in 20×SSC (3 M NaCl and 0.3 M Na citrate) for 10 min. The gel was then turned upside down onto a 3 MM Whatman paper which was used as a wick Any air bubbles were rolled out and a Genescreen® nylon membrane (DuPont, NEN, Boston, Mass., USA) of the same dimension was placed on the top of the gel to transfer the total RNA from the gel to the membrane overnight The nylon membrane was then carefully removed and exposed to UV light to crosslink the RNA to the membrane by using an energy level of 120 mJ in a UV Stratalinker® 1800 (Stratagene Corp., La Jolla, Calif., USA). The membrane was sealed in a plastic bag while the membrane was still moist.

Northern Blot Hybridisation

The blot was hybridised using the Hybaid® hybridisation bottle system with the Hybaid® hybridisation oven (Hybaid, Middlesex, United Kingdom) as this system gave sensitive and reproducible results. Before hybridising with the radiolabelled probe, the blot was soaked with 2×SSC (0.3 M NaCl and 0.03 M Na citrate) then prehybridised with 10 mL prehybridisation buffer containing 50% (v/v) deionised formamide; 0.8 M NaCl; 1 mM EDTA, pH 7.4; 50 mM PO₄, pH 6.5; 2% (w/v) SDS; 2.5× Denhardts solution (100× Denhardts solution consisting of 2% (v/v) Ficoll (Sigma), 2% (w/v) polyvinylpyrrolidone (Sigma) and 2% (w/v) bovine serum albumin); 100 mg/mL sheared salmon sperm DNA (Sigma); 200 mg/mL tRNA (last two reagents were denatured by heating to 95° C. for 5 min prior to addition) at 65° C. for 3 h with continuous rotation. This temperature (65° C.) was used for prehybridisation, hybridisation and washing to ensure high stringency conditions for annealing of probe to target RNA.

The radiolabelled cRNA probe (5×10⁶ counts/mL) was thawed quickly at room temperature and injected directly into the hybridisation bottle containing the prehybridisation buffer. Hybridisation was carried out with continuous rotation at 65° C. overnight Following hybridisation, the blot was washed twice in 100 mL of a buffer containing 0.1×SSC and 1% (w/v) SDS at 65° C. continuous rotation for 15 min. After washing, the moist blot was sealed in a plastic bag and exposed to a phosphorimager screen for between 24 h and 7 days. Scanning of the image was performed using the ImageQuant software program (Molecular Dynamics, USA).

cRNA Probes

Collagen Type II (HC22)

The cDNA was kindly supplied by Dr F Ramirez from the Brookdale Center for Molecular Biology, Mt Sinai School of Medicine, New York. The cDNA was 3.185% which encodes exons 21 to 52 of the human collagen type α-alpha1(II). The cDNA was subcloned into EcoR1 site of pBluescriptIISK (Stratagene). Antisense DIG and radioactively labelled cRNA probes were made by linearising the insert with BamHI and T7 RNA polymerase. Sense DIG and radioactively labelled cRNA probes were made by linearising the insert with HindIII and using T3 RNA polymerase.

Collagen Type IX (pKTh123)

The cDNA was kindly supplied by Dr Y. Ninomiya from the Department of Anatomy and Cellular Biology, Harvard Medical School, Boston, Mass., USA. The cDNA was 0.6 kb which encodes two-thirds of COL2 region through to half of NC2 region of human collagen type alpha1(IX). The cDNA was subcloned into EcoRI site of pBluescript (Stratagene). The insert was linearised with KpnI and T3 RNA polymerase was used to make antisense radioactively labelled cRNA probes.

Collagen Type X (NC1)

The cDNA was kindly supplied by Dr J. Bateman from the Department of Paediatrics, University of Melbourne, Victoria, Australia. The cDNA was approximately 0.7 kb which encodes the NC1 domain of human collagen type alpha1(X). The cDNA was inserted into the HindIII/SacI sites of pGEM7ZF(+) (Promega). The template was linearised with HindIII and SP6 RNA polymerase was used to make antisense radioactively labelled cRNA probes.

Aggrecan Probe (rpg4.16)

The cDNA was obtained from lgt11 library constructed from Swarm rat chondrosarcoma mRNA, kindly supplied by Dr K. Doege from the Research Department, Shriners Hospital, Portland, Oreg., USA (GenBank accession number J03485). The cDNA was approximately 1.6 kb which encodes the hyaluronic-acid binding region (G1 through half of G2). The cDNA was subcloned into EcoRI site of pBluescript (Stratagene). The insert was linearised with KpnI and T3 RNA polymerase was used to make antisense radioactively labelled cRNA probes.

Decorin Probe (P2)

The cDNA was kindly supplied by Dr Larry W Fisher from the Bone Research Branch, NIDR, Bethesda, USA. The cDNA was made from mRNA isolated from human bone cells and inserted into the EcoR1 site of pBluescript SK (Stratagene). The 1.6 kb insert contained the full sequence for coding human bone decorin. The template was linearised with BamH1 and T7 RNA polymerase was used to make antisense radioactively labelled cRNA probes.

Unknown Gene Product (DACC7)

A hybrid riboprobe (HC22pBluescriptIISK) was designed to screen a deer antler cDNA library (biased for highly expressed population) for collagen-like and abundantly expressed genes. All screened sequences were identified and sequenced, as described later. BLAST and FASTA analysis identified one unique insert (DACC-7) and found to be approximately 1 kb in length. Gene-specific primers were then designed from this sequence for 5′ RACE to obtain the 5′end of the DACC7 gene, which was sequenced and cloned as described later. The full sequence (1.474 kb) for DACC7 in pBK-CMV (Stratagene) was linearised with EcoRI and T7 RNA polymerase was used to make antisense DIG-labelled RNA probes. For sense DIG-labelled cRNA probes, the insert was linearised with-XbaI and using T3 RNA polymerase.

In Situ Hybridisation

DIG-labelling cRNA Probe Preparation

The DIG-Chem-Link labelling and Detection Set was purchased from Roche (Roche, Australia). The cDNA template was linearised with the appropriate restriction enzyme and 1 μg cDNA template was dried under vacuum. To the dried cDNA template, the following was added: 2 μL of 10×transcription buffer (400 mM Tris-HCl, pH 8.0; 60 mM MgCl₂; 100 mM dithiothreitol (DTT) and 20 mM spermidine); 13 μL of DEPC-treated water; 2 μL of 2.5 mM Nucleotide mix (10 mM rATP, 10 mM rCTP, 10 mM rGTP, 10 mM rUTP, pH 7.5); 2 μL of appropriate RNA polymerase (T7) and 1 μL of RNase Inhibitor. The mixture was briefly centrifuged then incubated for 2 hours at 37° C. The cDNA template was removed from the mixture after 2 hours incubation by directly adding 2 μL of DNaseI I and incubated at 37° C. for 15 minutes. In vitro transcription was stopped by adding 2 μL of 0.2 M EDTA (pH 8.0) solution The cRNA probe was then purified using Quick Spin Columns (Roche) as per manufacturer's instructions. The cRNA probe was eluted in 50 μL STE buffer (10 mM Tris, pH 8.0, 1 mM EDTA, 100 mM NaCl). The yield was measured by spectrophotometry, as described previously. The cRNA probe was then labelled with DIG using the DIG-Chem-Link labelling reagent as per kit instructions and stored at −70° C.

In Situ Hybridisation

The HC22 cRNA probe and DACC7 cRNA probe (1.474-kb unique sequence) expression localisation were compared by in situ hybridisation. The paraffin embedded tissue sections were deparaffinised in xylene and rehydrated in decreasing concentration ethanol solutions. The slides were immersed into xylene twice for 3 min and twice in 100% ethanol for three min. They were placed in 95% ethanol for 3 min and 70% ethanol for 3 min. Finally, the slides were immersed into DEPC-treated water for 3 min to complete rehydrating the tissue sections. The sections were then treated with 200 mM HCl at room temperature for 10 minutes to inactivate endogenous alkaline peroxidase and uncover the RNA from proteins. The slides were then washed 5 times in DEPC-treated water to remove the HCl. The sections were then incubated with agitation in 0.25% (v/v) acetic anhydride/0.1 M triethanolamine HCl/0.9% (w/v) NaCl buffer (pH 8.0) at room temperature for 10 min to bind positively charged molecules and protects RNA. The slides were again washed 5 times in DEPC-treated water to remove the acetic anhydride. The slides were initially placed in 95% ethanol, followed by 100% ethanol to dehydrate the tissue sections. Seventy microlitres of standard hybridisation buffer with 50% formamide (formamide, 50% (v/v); 5×SSC (0.1 M NaCl, 0.8 M NaCitrate, pH 7.0); 2% blocking reagent (Roche Kit) was placed on the slides to prehybridise at 55° C. for 2h in a humid chamber. After prehybridisation was complete, 65 μL of standard hybridisation buffer with 50% formamide containing 400 ng/mL of DIG-labelled cRNA probe was denatured at 80° C. for 5 min, then placed on the slides with coverslips. The sections were placed in a humid chamber and hybridisation was carried out overnight at 55° C.

The coverslips were carefully soaked off the slides by soaking for 30 min with 2×SSC at room temperature. Stringent washes were 55° C. for 1 h with 2×SSC, then twice at 55° C. for 30 min with 0.1×SSC. The slides were then equilibrated in TBST (Tris buffered saline with 0.3% Tween-20 (Sigma), pH 7.5) for 5 min in the Sequenza Immunostaining System (Shandon, UK), before incubating in 100 μL of 1:50 diluted antibody conjugate (rabbit F(ab) anti-DIG, alkaline phosphatase-coupled, Dako #D5105) in 0.5% (w/v) blocking reagent/TBST for 30 min at room temperature. The unbound antibody conjugate was removed by washing 5 min with TBST at room temperature. The slides were then removed from the Sequenza system and a Pap pen (Dako #S2002) was used to create a hydrophobic region around the tissue sections. The colour-substrate solution (5-Bromo-4-Chloro-3-Indoxyl Phosphate (BCIP)/Nitro Blue Tetrazolium Chloride (NBT) (Dako #K0598)) was added to slides to initiate colour development for the desired mRNA signal. The mRNA hybridised with the probe formed purple particles in the tissue sections. After the desired purple dots appeared on the slides and the colour reaction was stopped by washing the slides for 2 min with 50 m-L of DEPC-treated water. The slides were then mounted with Aquaperm Mounting Medium (IMMUNON™ Thermo, Shandon, Pa., USA), then a coverslip placed with Euckitt (O'Kindler GmbH and Co., Freiberg, Germany) and stored in the dark until analysed.

cDNA Expression Library Generation and Screening

cDNA Library Generation

An amplified lambda cDNA library was prepared from the first antler growth of a 2 year old Red deer stag (Cervus elaphus) using the ZAP-cDNA®/Gigapack® III Gold Cloning kit (Stratagene). All reagents were included in the kit unless otherwise stated and the kit protocol was strictly followed. This kit allows construction of directional cDNA libraries, therefore doubles the number of clones detectable by screening. It was designed for optimal library construction, including in vivo excision, eliminating subcloning procedures and the high-efficiency lambda system, increasing the size of the library, along with size exclusion providing a true representative cDNA library of the original population of mRNA. The representation has not been altered by PCR amplification, and only a single amplification of the library was performed. In brief, 375 μg of total RNA was extracted from the deer antler, devoid of skin, as described previously. A total of 5.175 μg of polyA RNA was extracted from this total RNA sample using a Dynabeads® mRNA Purification kit (Dynal Pty Ltd, Carlton South, Victoria, Australia). This kit purifies polyA RNA from total RNA using oligo (dT)₂₅ magnetic beads, so that ribosomal and transfer RNA were not included in the library. First strand cDNA was synthesised from this mRNA using the ZAP-cDNA® Synthesis kit (Stratagene). The double stranded cDNA was ligated into the lambda Uni-ZAP® XR vector using EcoRI (5′ end) and XhoI (3′ end) sites. This vector accommodates a DNA insert up to 10 kb in length.

The lambda library was then packaged using the Gigapack® III Gold Cloning kit (Stratagene) and the packaged recombinant lambda phage plated using the E. coli cell line XL1-Blue MRF. At this stage, titering of the primary library identified a recombinant titre of 7.98×10⁷ plaque forming units per μg vector arms. As primary libraries can be unstable, the library was amplified to obtain a more stable, higher titre stock The amplified library titred at 1.308×10⁹/mL. Detailed methodologies can be obtained from both the ZAP-cDNA® Synthesis kit and the Gigapack® III Gold Cloning kit (Stratagene).

Preparation of cDNA Library Filters for Screening

For screening the cDNA library, large 135 mm plates were used to achieve-approximately 50,000 plaques per plate. Once the plaques had formed, the plates were maintained at 4° C. for approximately 2 h to allow the top agarose to harden before filter lifting. Colony/Plaque Screen™ membranes (DuPont) were labelled on the tab and placed face down on the plate for 2 min, during which time the orientation holes were marked on the bottom of the plate. When duplicate plaque lifts were performed, then the second filter was in contact with the plate for a duration of 5 min to allow efficient transfer. Using a plate lid as a dish, 3MM paper covering the bottom was saturated with a denaturing solution (1.5 M NaCl, 0.5 M NaOH) and the filter was placed plaque side up in this solution for 2 min. The filter was then dragged along the lip of the tray to remove excess solution and placed in a second tray saturated with a neutalising solution (1.5 M NaCl; 0.5 M Tris HCl, pH 8.0) for 5 min. This process was repeated with a rinse solution (0.2 M Tris HCl, pH 7.5; 2×SSC) for 30 sec. The filter was then blotted between sheets of 3MM paper and exposed to an energy level of 120 mJ in a UV Stratalinker 1800 (Stratagene) to crosslink the DNA to the filter. While still moist, the filter was sealed in a plastic bag and stored until ready to hybridise.

Screening a cDNA Library with Riboprobes

cDNA filters were prehybridised either back to back or between mesh in the hybridisation bottle system (Hybaid) when more than 2 filters screened at one time. Prehybridisation, hybridisation and washing were performed as for Northern blot hybridisation as described previously.

Hybridisation marks on the filter image corresponding to plaques were cored and a secondary screening was performed. Clones surviving the second screening underwent a final tertiary screening before consideration for further characterisation. Any clones that survived this screening procedure were in vivo excised.

In Vivo Excision

The design of the Uni-ZAP® XR vector (Stratagene) allowed in vivo excision and recirculation of the cloned cDNA insert contained within the lambda vector arms to form a plasmid containing the cloned insert. As a plasmid, the cloned cDNA could be stored as a glycerol stock, and mini DNA preparations could be performed. Thus, any cored plaque of interest was in vivo excised for further characterisation. The methodology for in vivo excision could be found in the protocol for the ZAP-cDNA® Synthesis kit (Stratagene) which also contained the required reagents.

Mini-Preparation of Plasmid DNA

Mini-preparation of plasmid DNA was prepared by using the Wizard® Plus Miniprep DNA Purification system (Promega) as described previously. Because of the poor yields of the plasmid DNA it was necessary to transform the plasmid into another host, DH5α, to obtain better quality DNA (as described later). Clones were sequenced and also reassessed using PCR techniques to help characterise the clones (as described later). Clones of interest were selected for midiprep DNA extraction as described previously.

5′ RACE (Rapid Amplification of cDNA Ends), Cloning and Sequencing 5′ RACE

5′ RACE is a procedure for amplification of nucleic acid sequences from a messenger RNA template, between a defined internal site and unknown sequences at the 5′ end of the mRNA. This technique was used to obtain the 5′ end of the DACC7 gene using sequence information provided from the partial 3′ DACC7 clone obtained from screening of the cDNA library to generate DACC7GSP1 and nested DACC7GSP2 gene-specific primers for 5′ RACE.

A 5′ RACE System of Rapid Amplification of cDNA ends, Version 2.0 kit (Gibco BRL, Life Technologies) using 4 protocols was applied on 2 year old Red deer stag DAC RNA as per kit instructions. Gene specific primer 1s' (GSP1s) were designed based on kit instructions and the 5′ end of the cDNA library clone sequences. For example, DACC-7 GSP1 (primer for 1^{st} strand synthesis), was a 20-mer with a melting temperature of 63° C. and consisted of 5′ GTT CCA CAC GTC ACC ACA GT 3′ (SEQ ID NO: 39).

Advantage® cDNA PCR kit (CLONTECH Laboratories, Inc., USA) was used in Protocol 4 of the 5′ RACE System using the following cycles: 94° C. for 1 min; a step cycle of 94° C. for 0.5 min, 60° C. for 1 min and 72° C. for 5 min for 35 cycles; followed by 72° C. for 7 min to allow final extension. The Abridged Anchor Primer and a nested GSP2 were used in the PCR. GSP2s were nested primers in reference to the GSP1s, designed from the cDNA library clone sequences as per kit instructions. For example, DACC-7 GSP2 (primer for PCR) was a 24-mer with a melting temperature of 60° C. and consisted of 5′ CGT ATC GTG CTT AAA TAT GTC AGT 3′ (SEQ ID NO: 40).

Cloning Techniques

Cloning of PCR Products

Cloning of PCR products into pGEM-T Easy® Vector was achieved using a pGEM-T Easy® Vector Systems Kit (Promega). A 1:1 insert: vector molar ratio was used and the ligation reaction was incubated at 4° C. overnight as kit instructions.

Transformation of Cloned PCR Products into JM109 Competent Cells

The cloned PCR products were transformed into JM109 High efficiency competent cells (Promega) as per kit instructions.

Restriction Cloning of DNA

Non-PCR DNA products to be cloned were restriction digested with appropriate enzymes to create overhanging “sticky ends” that were compatible with overhanging ends of similar digested vector. Each restriction digest was gel purified before undergoing the ligation reaction.

For each insert to be ligated, both 1:1 and 3:1 insert:vector molar ratio reactions were carried out using the formula:

ng of vector×sizE(kb) of insert×insert:vector molar ratio=ng of insert sizE(kb) of vector

For every 10 μl of reaction, 1 μL of 10×ligation buffer (Roche Molecular Systems), 1 μof T4 DNA ligase (Roche Molecular Systems) and 1 μL of glycogen (which increases the probability of T4 ligase molecules coming into contact with overhanging DNA ends) were combined with the appropriate amounts of vector and insert in 0.5 mL microcentrifuge tubes. After brief mixing and spinning in a microfuge the ligation reaction was incubated at 4° C. overnight.

Preparation of Competent Cells

To prepare competent cells for transformation, 50 mL of Luria-Bertaini (LB) medium (10 g tryptone, 5 g yeast extract, 10 g NaCl) was inoculated with 0.5-1.0 ml overnight culture (DH5a E. coli strain) in a 250 mL conical flask and cultured for 3-4 h at 37° C. with shaking until the OD₆₀₀ reached 0.5. The cells were chilled on ice for 20 min before spinning at 3,000 rpm for 10 min to pellet the cells. 5 mL cold (4° C.) CaCl₂ was added to reused the bacteria. The cells could be used immediately for transformation or aliquoted and stored at −70° C.

Transformation of Ligation Reactions by Electroporation

For each transformation reaction, 2 μL of overnight ligation reaction was combined with DH5a competent cells in a sterile 1.5 mL microtube and mixed by flicking. The mixture was incubated on ice for 20 min, after which they were heat-shocked at 42° C. in a water bath for 45 s and immediately returned to ice for 2 min. LB medium was added to the tubes and incubated at 37° C. with shaking for 1.5 h. The transformations were then plated onto LB plates containing 100 μg/mL ampicillin and incubated at 37° C. overnight

Cloning of a Putative Full Length DACC7

The expression vector pBK-CMV (Stratagene) is a useful vector for recombinant protein expression. The vector allows expression in both eukaryotic and prokaryotic systems. Eukaryotic expression is driven by the cytomegalovirus (CMV) immediate early promoter. Stable clone selection in eukaryotic cells is made possible with G418 by the presence of the neo-mycin-and kanamycin-resistance gene, which is driven by the SV40 early promoter with thymidine kinase (TK) transcription termination and polyadenylation signals.

The expression vector pBK-CMV was modified to remove the prokaryotic lac promoter and lacZ translation start site, since this results in increased eukaryotic expression, essential for protein function studies. This construct was named pBK-CMV.2. A full length DACC7 contig was pieced together using carefully chosen restriction enzymes as outlined in FIG. 27 using methods described previously. This was cloned into pBK-CMV.2 but was not fused in frame to the lacZ gene and contained non-coding 3′end sequence. The cloning steps and transformations were carried out as outlined previously into DH5α or JM109 competent cells. To generate plasmid DNA after each cloning step, a miniprep was carried out and restriction digested to obtain plasmid DNA for the next cloning step.

Sequencing of DNA

Miniprep plasmid preparations (described previously) of cloned PCR products were sequenced using T7 and SP6 primers. Sequencing was done by AGRF.

Histochemical and Immunohistdchemical Methods

Histological Staining

Deer antler cartilaginous tips were divided into the 3 zones shown in FIG. 21 and each zone subdivided into two equal parts. One-half was immediately fixed in 10% neutral buffered formalin, the other in Histochoice fixative. The fixed tissues were embedded in paraffin and 5 μm histological sections cut and mounted using standard techniques. The formalin-fixed sections were processed and stained with haematoxylin and eosin or 1% (w/v) Toluidine Blue at pH 1.0 and 2.5 respectively, then counter-stained with fast red dye, as described in detail by Little et al. (1997).

Immunohistochemical Staining

For these studies the antler tissues fixed in Histochoice (Amresco #H102-IL, OH, USA) were used. Histochoice is a fixative which does not contain formaldehyde, thereby eliminating the need for recovery of the target and predigestion of paraffin sections. The immunolocalisation of type II collagen was undertaken essentially as described previously (Little et al. 1997) but with the following modification. Glass mounted cut sections were incubated at 4° C. for 16 h and treated with a commercially available monoclonal antibody (Anti-human type II collagen, purified mouse IgG1, Clone: II-4C11, titre: 500 μg/mL, 1:50 dilution (ICN Biomedicals, OH, USA)). A biotinylated secondary antibody (anti-mouse/rabbit immunoglobulin (Dako LSAB₂, K1015) was added for 30 min at 20° C. then peroxidase-labelled streptavidin (Dako LSAB+peroxidase K0690) for 30 min at 20° C. Staining was completed following incubation with Nova Red (Vector Laboratory SK-4800) substrate solution and rinsing.

Tissue and Cell Preparations for Cell Culture Studies

Deer Antler Cartilage (DAC) Used for Alginate Bead Cultures

The cartilaginous tips from 3 mature fallow deer stags (Dama dama, designated F1, F2, F3) were collected during the maximal growth period under local anaesthetic (Lignocaine) as described previously for RNA preparation. A section of the cartilage centre was removed for histological examination as shown in FIG. 3. The remaining deer antler cartilage (DAC) was separated into 3 zones (A, B, C) as shown in FIG. 3, corresponding to the prechondrocytes region (zone A), mature proliferating chondrocyte region (zone B), and hypertrophic chondrocyte region (zone C). The predominant chondrocyte populate in these zones were confirmed by the corresponding histological assessment The DAC zones were discernible morphologically as the prechondrocyte tissue which was observed as a white, soft cartilage with no blood vessels; the mature chondrocyte tissue observed as soft cartilage with blood vessels; and the hypertrophic chondrocyte tissue which showed encroaching mineralisation and blood vessels invasion. Since the 3 zones merged with each, pure cell population from each could not be obtained. The outer rim of cartilage in each DAC zone was discarded, DAC cells from the 3 zones (A, B, and C) were released by enzymatic digestion as described previously for RNA preparation. Their viability was determined by dye exclusion using a haemocytometer.

DAC Used for Monolayer Cultures

Antler specimens were collected from 2 fallow deer (F4, F5) and red deer (Cervus elaphus) designated deer 6-antler 1 (R6.1), red deer 6-antler 2 (R6.2). Tips of these specimens were dissected as shown in FIG. 3 and cells released as described previously.

Sheep Articular Chondrocytes (SAC) Used for Monolayer Cultures

Sheep articular chondrocytes (SAC) were obtained from the stifle joints of 4-year-old purebred Merino sheep. Joints were transported to the laboratories on ice within 4 h of sacrifice, were opened under sterile laboratory conditions and full-depth articular cartilage was sliced from the tibial plateaux (TP) and the femoral condyles (FC) including the trocheal groove using a #11 blade. Each cartilage area (TP or FC) was enzyme digested with 0.1% (w/v) pronase (Boehringer Mannheim Australia Pty. Ltd., Castle Hill, NSW, Australia) in DMEM:F12 media containing 10% (v/v) FBS at 37° C. for 2 h then changed to 0.04% (w/v) collagenase in DMEM:F12/10% (v/v) FBS for digestion overnight at 37° C. to release the cells. Cells were collected through a sterile 70 μm cell strainer and viability determined by dye exclusion using a haemocytometer.

Rabbit Ear Chondrocytes (REC) Used for Monolayer Cultures

Rabbit ears were dissected from a New Zealand male rabbits and cartilage obtained by meticulously removing the skin and periosteum under sterile conditions in a laminar flow cabinet The diced ear cartilage was enzyme digested with 0.125% (w/v) trypsin in DMEM:F12 at 4° C. overnight, then 37° C. for 1 h. This was replaced with media containing 0.04% (w/v) collagenase and supplemented with 10% (v/v) FBS at 37° C. for 5 h, vortexing for 10 sec every 30 min. REC were collected through a sterile 70 μm cell strainer and viability determined by dye exclusion using a haemocytometer.

REC Used for Explant Cultures

REC was collected as described above, except that there was no digestion step, instead the prepared cartilage was diced into explants (approx. 1 mm²) and used directly for culture experiments.

Cell Culture Methods

DAC Alginate Bead Cultures

DAC bead cultures were prepared essentially as described by Häuselmann et al. (1994). Briefly, for each zone (A, B, C) DAC cells obtained after collagenase digestion were centrifuged and washed twice with DMEM:F12. The cell pellets were re-suspended at a density of 3×10⁶ cells/mL in alginate solution which contains 1.2% (w/v) sodium alginate (Sigma) dissolved in 0.15M NaCl (Ajax Chemicals, Auburn, NSW, Australia). The cell suspension was slowly expressed through a 23-gauge-needle and the droplets formed allowed to fall into a 100 mM CaCl₂ (May and Baker Australia Pty. Ltd., Australia) solution. The beads (20,000 cells/bead) were allowed to polymerise in this solution for 10 min. They were then transferred to a 48 (Costar, Cambridge, Mass., USA), (10 beads/well) or 96 (Greiner, Maybachstrasse, Frickenhausen, Germany), (2 beads/well) well plates and covered with DMEM:F12/10% (v/v) FBS medium. After 24 h incubation at 37° C. in an atmosphere of 5% CO₂/95% air with 75% humidity, DAC conditioned media (DAC-CM) was collected from each well.

DAC Monolayer Cultures

DAC cells prepared as described previously were seeded into 75 cm² flasks culture flasks at 2×10⁶ cells/mL by incubating in DMEM:F12 media with and without 10% FBS at 37° C. in an atmosphere of 5% CO₂/95% air with 75% humidity. DAC CM was collected from each primary culture (i.e. media was replaced but the cells were not subcultured) at specified time points. DAC-CM samples were prepared from specimens F4, F5, R6.1 and R6.2 and collected on days 1, 3, 5, 7, 9, 11, 13 and 18 post-culture initiation.

SAC Monolayer Cultures

SAC were cultured as monolayers at 1×10⁵ cells/mL in 75 cm² flasks (Corning) with DMEM:F12 media containing 10% FBS at 37° C. in an atmosphere of 5% CO₂/95% air with 75% humidity. Once confluence was reached, SAC were treated with various concentrations of DAC-CM obtained from DAC bead culture experiments from zones A, B and C collected after 24 h. DAC-CM concentrations used were 1, 3, 10, 30, 100% (v/v) or control media [DMEM:F12/10% (v/v) FBS]. These experiments were used to determine DAC cell zonal synthesis of DNA and total proteoglycan (PG) synthesis.

REC Monolayer Cultures

REC were cultured as monolayers at 5×10⁴ cells/ml in 75 cm² culture flasks with DMEM:F12 media containing 10% (v/v) FBS at 37° C. in an atmosphere of 5% CO₂/95% air with 75% humidity. Once confluence was reached, REC were treated with 50% DAC-CM from DAC bead or monolayer cultures, i.e. A, B and C collected after 24 h and F4, F5, R6.1, R6.2 media colleted 1 d, 3 d, 5 d, 7 d, 9 d, 11 d, 13 d and 18 d post-culture initiation.

REC Explant Cultures

Diced (˜1 mm×1 mm) explants of REC were cultured (4 explants/well) with DMEM:F12 media containing 10% (v/v) FBS at 37° C. in an atmosphere of 5% CO₂/95% air with 75% humidity. The media was removed and REC cells were treated with DAC-CM from DAC bead culture obtained from regions A, B and C per 1 d or from CM from cultures from F4, F5, R6.1, R6.2 collected at 1 d, 3 d, 5 d, 7 d, 9 d, 11 d, 13 d and 18 d post-culture initiation.

Mouse Fibroblast Cell Line

A 3T3 Swiss Albino P137 contact inhibited cell line (CSL, Victoria, Australia, ATCC CCL 92) was used for the growth factor assay, as described by Klagsburn et al. (1977). 3T3 cells were cultured in 96-well plates (5×10⁴ cells/mL, 1×10⁴ cells/well) in DMEM:F12/10% (v/v) FBS at 37° C. in an atmosphere of 5% CO₂/95% air with 75% humidity. The media was removed and 3T3 cells were treated with DAC-CM from DAC bead culture obtained from regions A, B and C per 1 d or from CM from cultures from F4, F5, R6.1, R6.2 collected at 1 d, 3 d, 5 d, 7 d, 9 d, 11 d, 13 d and 18 d post-culture initiation.

Assay for Biosynthesis of Proteoglycans

DAC Alginate Bead Cultures

Alginate beads from each DAC zone (A, B and C) were placed in 48-well plates (10 bead/well) and incubated with DMEM:F12 media containing Na₂³⁵SO₄ (Amersham, Cardiff, UK) added (5 μCi/well) for 8 h, 24 h, 48 h and 72 h. At the termination of the incubations media and alginate beads were processed separately (4 replicates) at each time-period. Alginate beads and their respective media were individually digested with papain (Sigma) (50 μh/mL in PBS containing 10 mM EDTA and 5 mM cysteine) at 60° C. for 2 h and unincorporated ³⁵SO₄ was removed using BaSO₄ precipitation as previously described by Collier and Ghosh (1989). Briefly, an aliquot of the papain digested sample (400 μL) was mixed with a solution of −0.1 M NaSO₄ containing 25 mg/mL chondroitin sulphate (Sigma Chemical Co.) (200 μL). To this solution was added 100 μL of 0.4M BaCl₂. The sample was vortexed, centrifuged (2500×g) and a 400 μL aliquot of the supernatant collected and the above described precipitation repeated. A 250 μaliquot of the supernatant from the second precipitation was collected and 50 μL of 1.1 mg/mL chondroitin sulphate in 0.3M BaCl₂ added. The sample was vortexed, 50 μL of 0.2M Na₂SO₄ solution was added and the sample vortexed again and centrifuged as before. A 100 μaliquot of this supernatant was collected, mixed with 5 mL scintillant (Emulsifer Safe®, Canberra Packard, Gladesville, NSW, Australia) and the radioactivity determined by liquid scintillation spectrophotometry (Model 1500 Liquid Scintillation Analyser, Canberra Packard) and the disintegrations per minute (DPM) determined for 2 min. The DPM of each 100 μL sample were multiplied by the dilution factors inherent in the assay to give a total DPM per sample.

Effects of DAC-CM on SAC Synthesis of Proteoglycans in Monolayer Cultures

Cells isolated from the TP or FC of sheep joints were cultured in 24-well plates (Nunc, Denmark), 60,000 cells/well. DAC-CM from zones A, B or C region at concentrations 1, 3, 10, 30, 50 and 100% (v/v) or control (DMEM:F12/10% (v/v) FBS) all containing Na₂³⁵SO₄ (5 μCi/well) were added to the wells. After 48 h incubation the media and cells were collected separately, papain digested and ³⁵S-labelled PGs isolated-and counted, as described previously.

Effects of DAC-CM on REC Synthesis of Proteoglycans in Explant Cultures

REC explants were placed in 24-well plates (4 explants/well). To some wells DAC-CM diluted to a concentration of 50% (v/v) with DMEM:F12/10% (v/v) FBS, and Na₂³⁵SO₄ (5 μCi/well) were added. Control wells contained only DMEM:F12/10% FBS and Na₂³⁵SO₄ (5 μCi/well). After 48 h incubation the media and explants were collected separately, papain digested and ³⁵S-labelled PGs isolated and counted as described previously.

Assay for DNA Synthesis

DAC Alginate Bead Cultures

DNA synthesis of DAC cells in alginate beads were determined using the assay described by Hutadilok et al. (1991) with the modification that the beads were dissolved as described by Häuselmann et al. (1994). Briefly, for each DAC zone (A, B, C), alginate beads (2 beads/well) were placed in 96-well plates. After 24 h incubation, media was changed and ³H-thymidine added (0.5 μCi/well). After 8, 24, 48 and 72 h incubation with ³H-thymidine (5 replicates), media was discarded, beads dissolved in NaCl (Häselmann et al. 1994) and cells collected using a cell harvester (Titertek Plus) onto glass filter paper (ICN Biomedicals, Costa Mesa, Calif., USA). The incorporated radioactivity into DNA was determined by liquid scintillation spectrophotometry (Model 1500 Liquid Scintillation Analyser) by mixing 3 mL scintillant with the glass filter paper and DPM counted for 2 min. Results were expressed as DPM/well (mean±sem).

SAC Monolayer Cultures

SAC from the TP or FC were cultured in 96-well plates (15,000 cells/well). DAC-CM from zones A, B or C region at concentrations 1, 3, 10, 30, 50 and 100% (v/v) or controls containing no DAC-CM [DMEM:F12/10% (v/v) FBS] plus ³H-thymidine (0.5 μCi/well) were added to each well. After 24 h incubation, ³H-thymidine-labelled DNA was determined as described previously.

REC Monolayer Cultures

REC were cultured in 96-well plates (10,000 cells/well) with media containing DAC-CM at 50% (v/v) concentration or controls containing no DAC-CM [DMEM:F12/10% (v/v) FBS] plus ³H-thymidine (0.5 μCi/well). After 24 h incubation, ³H-thymidine-labelled DNA was determined as described previously.

3T3 Mouse Fibroblast Cultures

3T3 cells were incubated with media containing DAC-CM from zones A, B or C at 50% (v/v) concentration or with control containing no DAC-CM [DMEM:F12/10% (v/v) FBS]. ³H-thymidine (0.25 μCi/well) was added to each well and after 3 h incubation, media was removed, cells were harvested and ³H-thymidine-labelled DNA levels determined as described previously.

Metabolic Activity of Cells Using the MTT (3-[4,5-dimethylthiazol-2-yl]-2,5-diphenyl-tetrazolium bromide) Assay

SAC Monolayer Cultures

Cellular metabolism of SAC (mitochondrial dehydrogenase activity) in the absence and presence of DAC-CM was determined using the assay method described by Mosmann (1983) but with the following modifications: SAC TP or FC were incubated for 24 h in 96-well (15,000 cells/well) with DMEM:F12/10% FBS or 1, 3, 10, 30, 50 and 100% (v/v) of DAC-CM from each zone. MTT (10 μL, 5 mg/mL in PBS) was added to each well and the plates were incubated for a further 2 h at 37° C. Media was removed and 100 μL (w/v) SDS in 55 mM Na-citrate/150 mM NaCl was added to each well to dissolve the crystals. Colour development in wells was then read in the Thermomax microplate reader (Molecular Devices, Menlo Park, Calif., USA) set at a wavelength of 562 nm.

Statistics

The Student's t-Test was used to determine whether two means from individual samples were significantly different, where p<0.05.

Proteomics

This part of the analysis was facilitated by access to the Australian Proteome Analysis Facility established under the Australian Government's Major National Research at Macquarie University.

Samples of conditioned media from alginate bead cultures from antler of F4 and F5 fallow deer were collected at 24 h and 7 d (168 h) after initiation of cultures. Each supernatant sample was submitted to amino acid analysis to determine the protein content of each sample. This analysis showed that sample 1 (F4-24 h) had 1.49 mg/ml, sample 2 (F4-168 h) had 1.14 mg/ml, sample 3 (F5-24 h) had 1.15 mg/ml and sample 4 (F5-168 h) had 0.61 mg/ml of protein. Samples underwent TCA precipitation to purify proteins, then were solubilised with sonication for 30 s. Endonuclease was added and samples were then centrifuged at 20,000×g for 10 min. Samples were then loaded onto gels for Isoelectric Focusing (IEF). For the range pH3-6 and pH5-8 gradient strips were loaded via in-gel rehydration; for pH6-11 gradient strips were cup loaded at the anode. For first dimension IEF, 95,000 Vh separating gel gradient 8-18% T large format polyacrylamide slab gels were used, while for second dimension electrophoresis, 6 h @ 3 mA/gel 14 h @ 15 mA/gel conditions were employed. Gels were stained with SYPRO Ruby fluorescent stain, scanned to produce a digital image and the resultant sample images were compared using Z3 Image Analysis Software (Compugen). The triplicate images from each of the culture supernatants were used to compile a raw master reference gel composite. The 3 composite gels generated for each sample were then used to compare protein profiles between culture supernatants. This was done for pH3-6, pH5-8 and pH6-11 gradients. The acquired image analysis data was then used to identify potential targets for a 16 h protein tryptic digest at 37° C. The resulting peptides were purified using a ZipTip to concentrate and desalt the sample. The samples were then analysed by ESI-TOF MS/MS using a Micromass Q-TOF MS equipped with a nanospray source and data manually acquired using borosilicate capillaries. Data was acquired over the m/z range 400-1800 to select peptides for MS/MS analysis. After peptides were selected, the MS was switched to MS/MS mode and data collected over the m/z range 50-2000 with variable collision energy settings.

Results

cDNA Sequences Overexpressed in Antler Cartilage Cells

The present invention is based on the unexpected and surprising discovery that chondrocytes of rapidly growing cartilage of regenerating deer antler express unique genes products which are not expressed in articular cartilage or epiphyseal growth plate chondrocytes of adult or full-term foetal deer, ovine or human cartilages. Of even greater surprise is the finding that several of these gene transcripts, seen as separate band on Northern Blot analysis, are only expressed in the early stage of chondrogenesis in human foetal tissues.

In the use of various collagens as antigens for treatment of arthritic diseases by oral tolerance which is mediated by T-cells, type II collagen from bovine origins is known to be less effective than type II collagen derived from the chick (Cremer et al. 1992; Zhang et al. 1990; Hart et al. 1993; Myers et al. 1993; Weiner et al. 1994; Barnett et al. 1996; Trentham et al. 1993; Sieper et al. 1996).

The present invention has demonstrated the cloning of a novel gene using deer antler cartilage as the starting material. A full-length clone was obtained by screening a cDNA library and by applying the technique of 5′ RACE. The pattern of expression of the gene was examined in human tissues at the mRNA level and the human chromosomal localisation of this novel gene was also established.

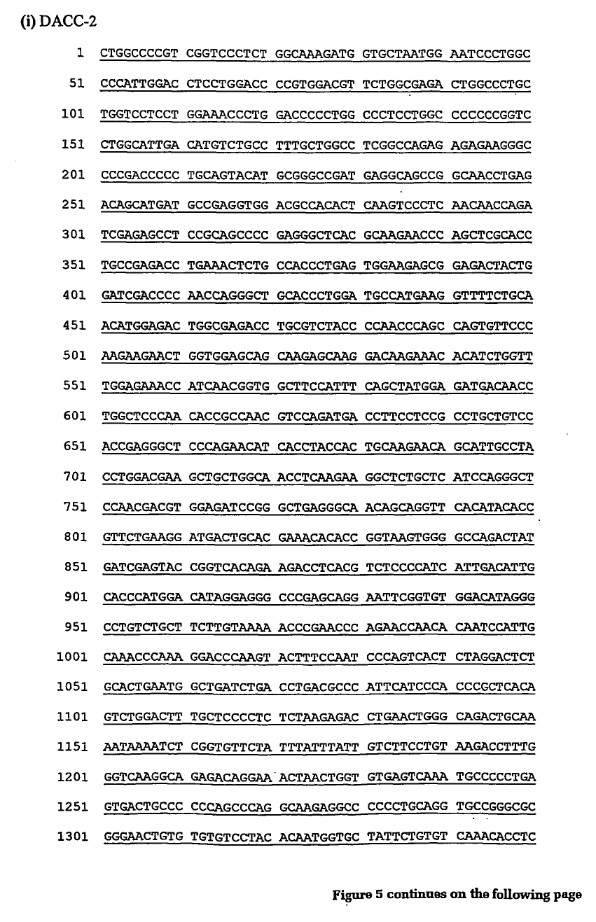

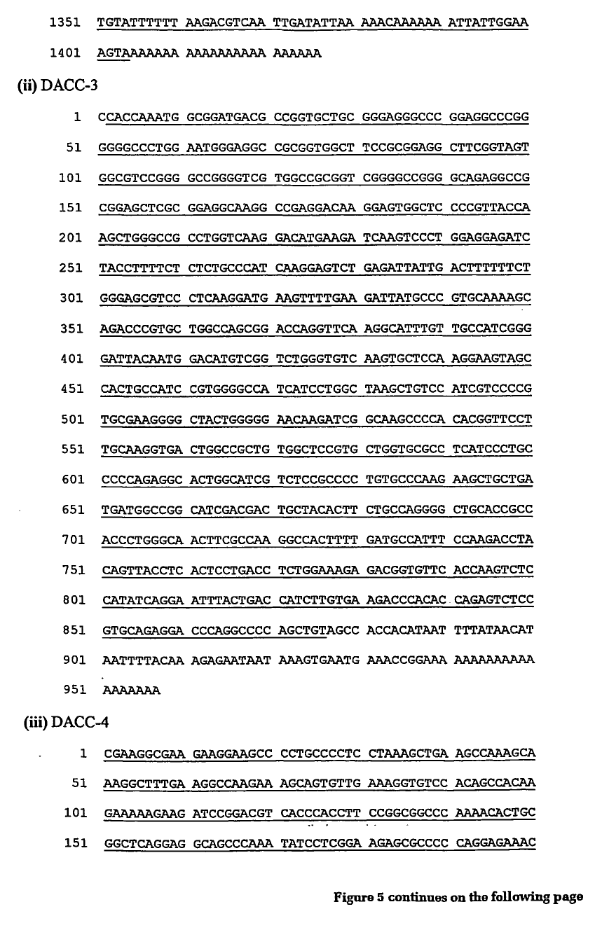

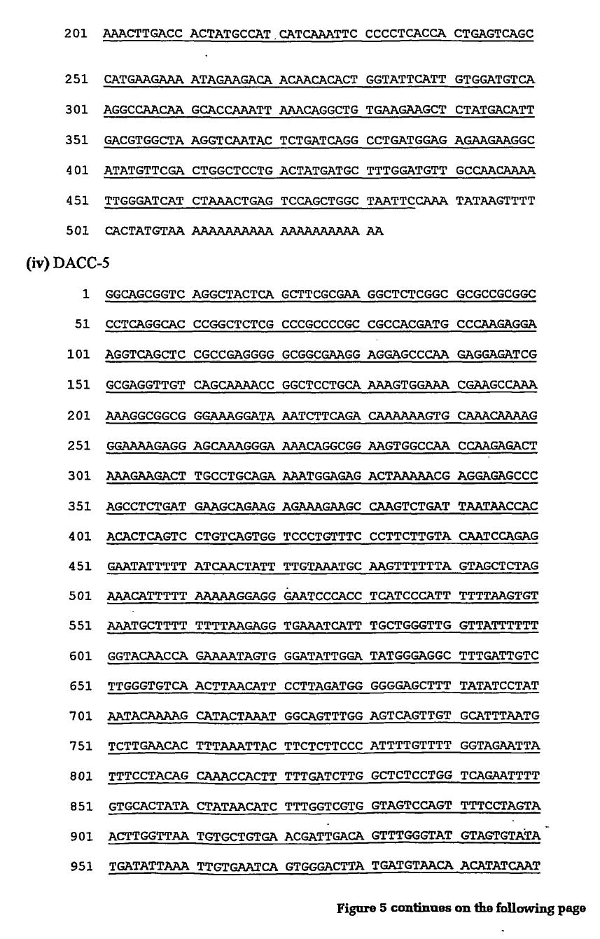

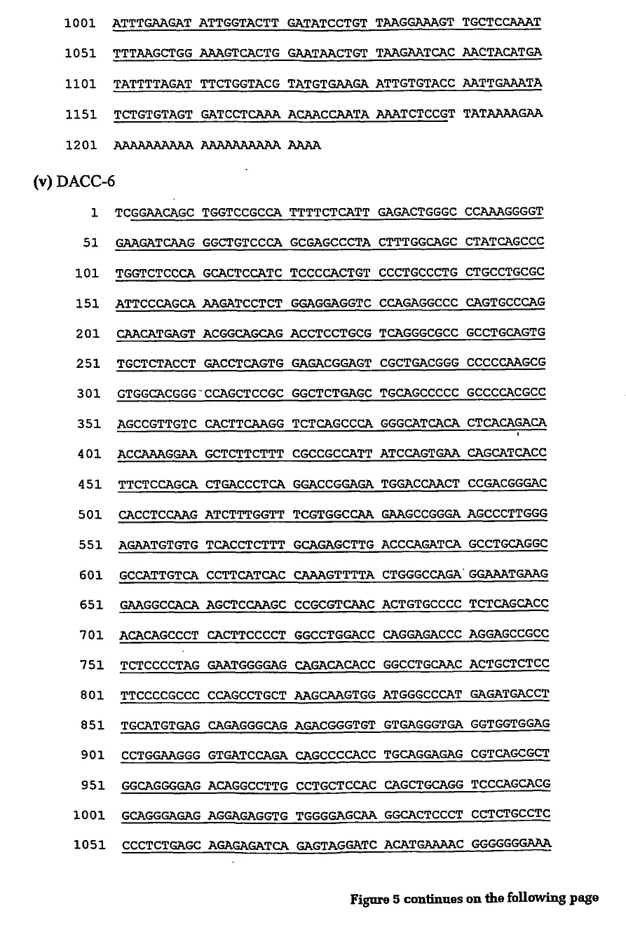

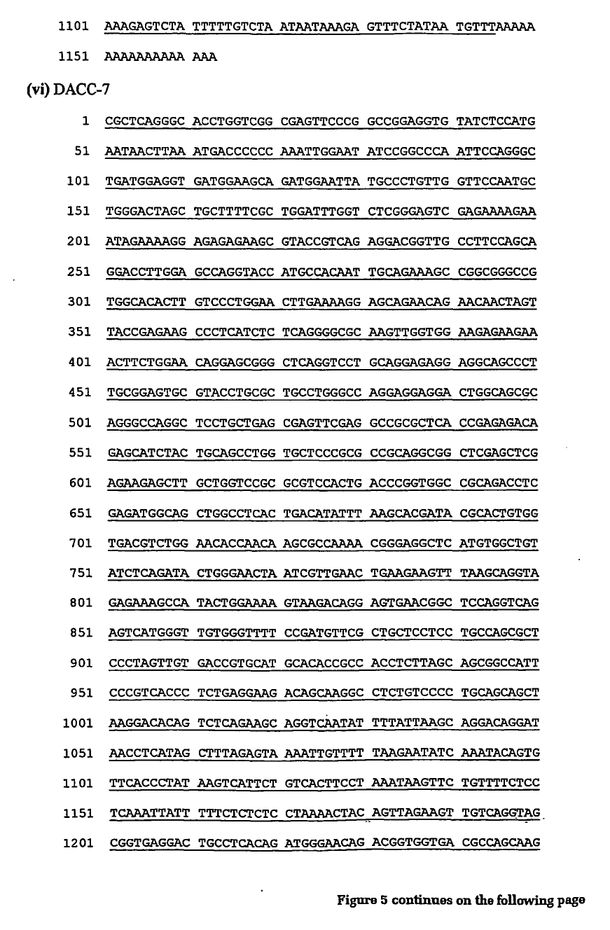

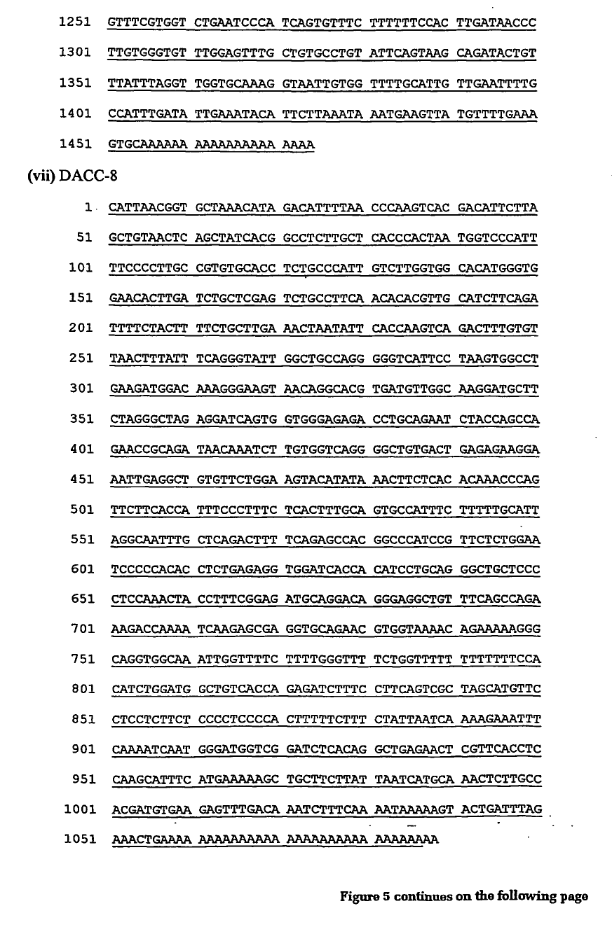

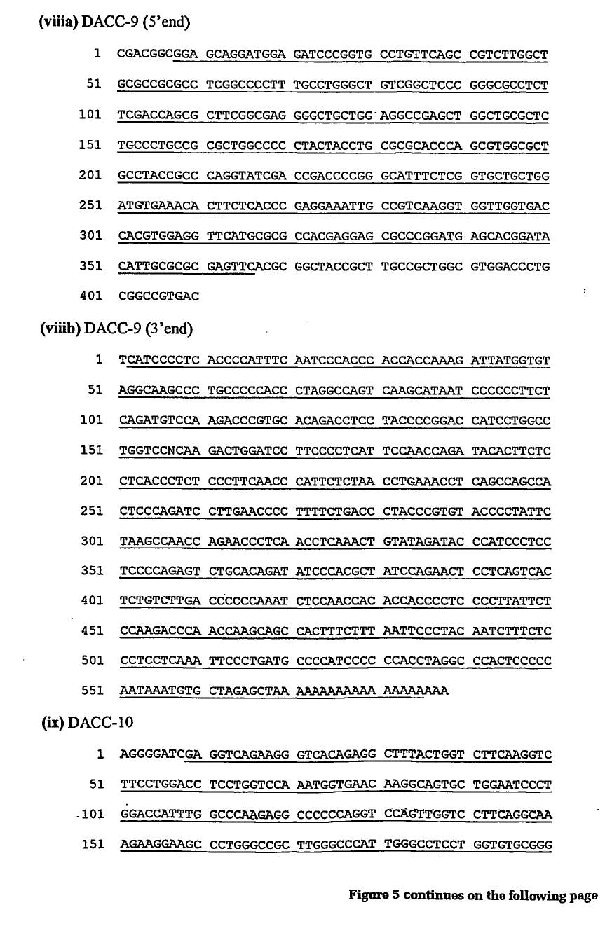

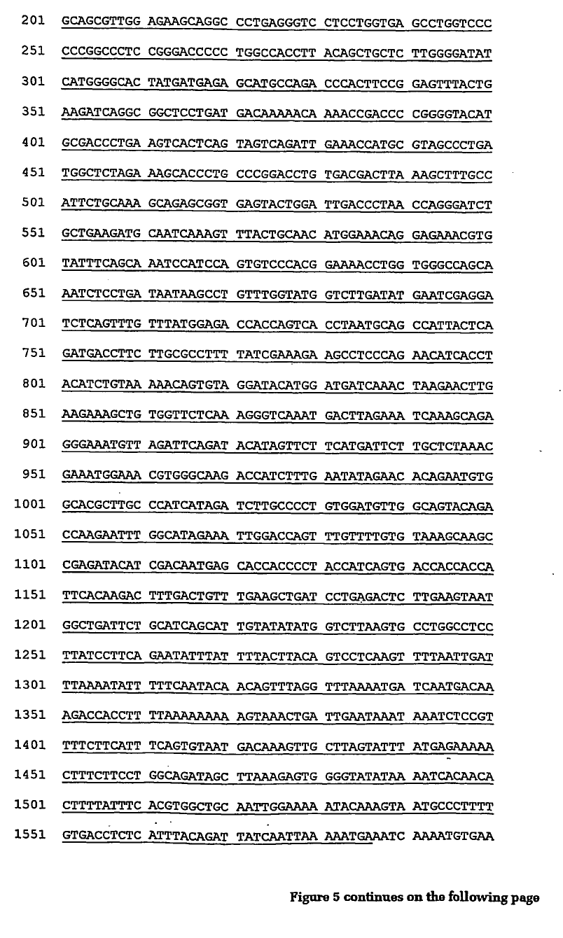

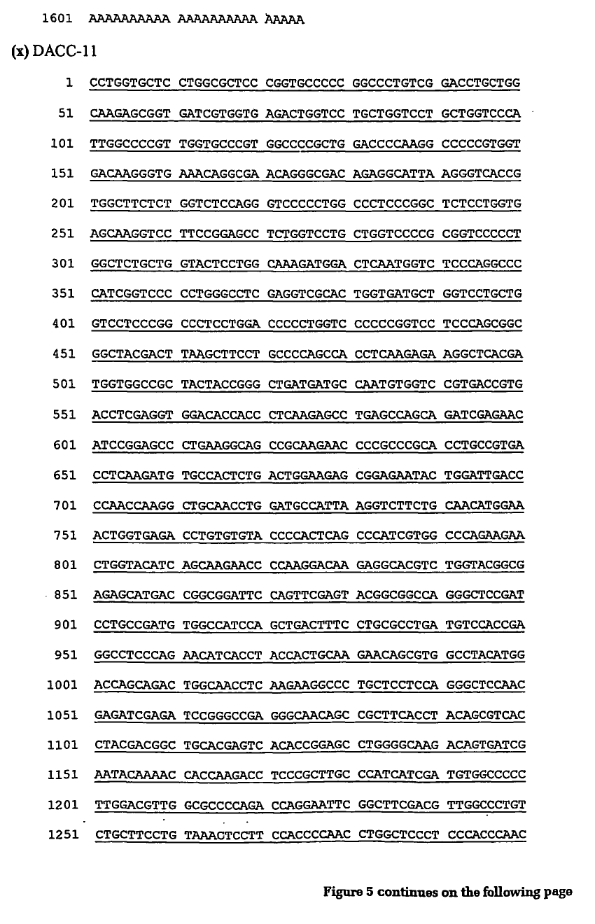

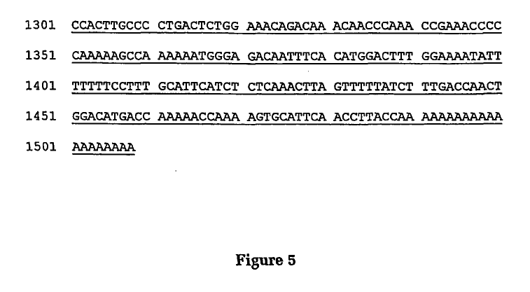

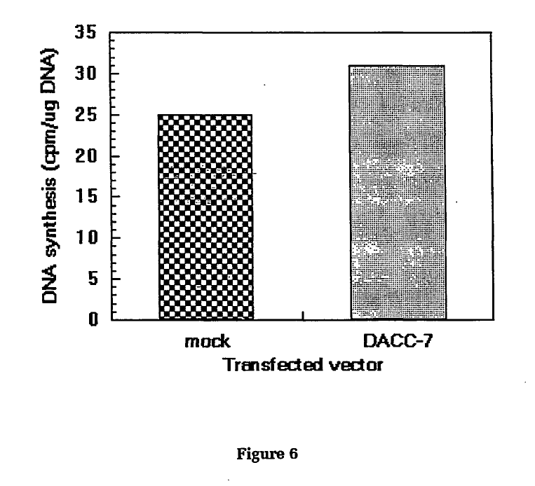

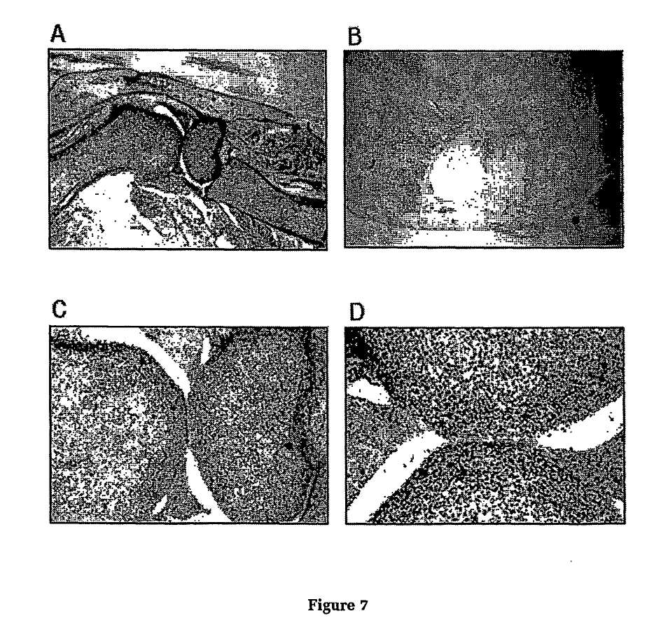

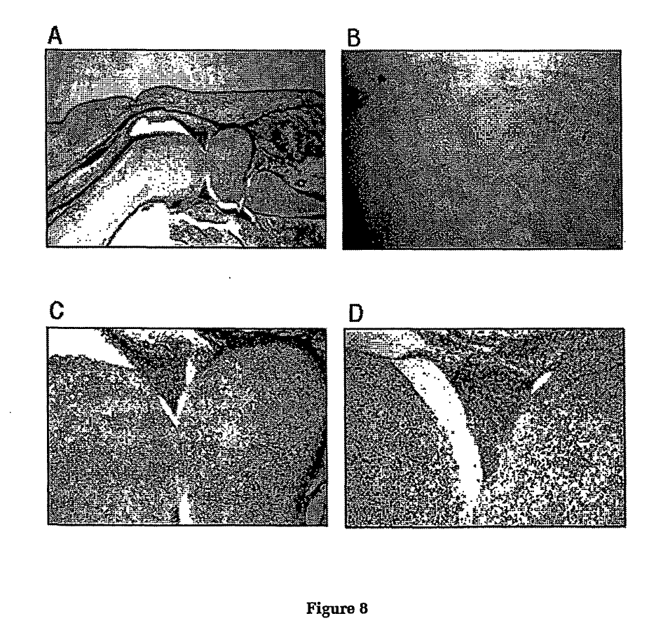

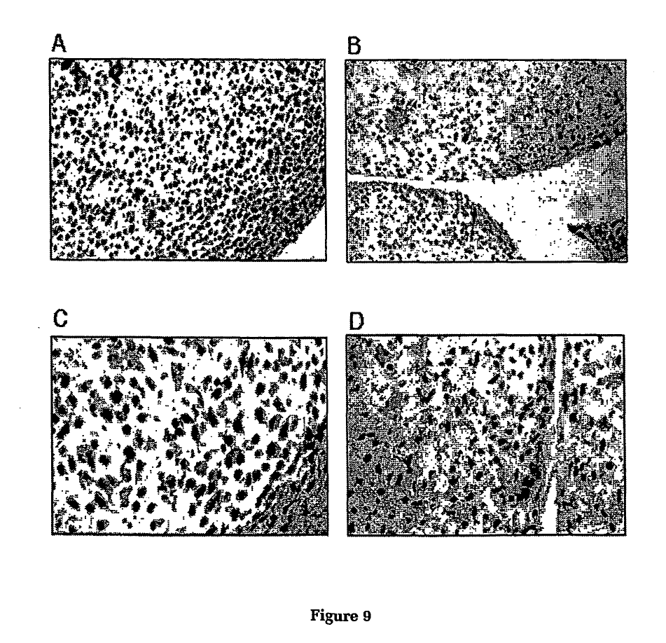

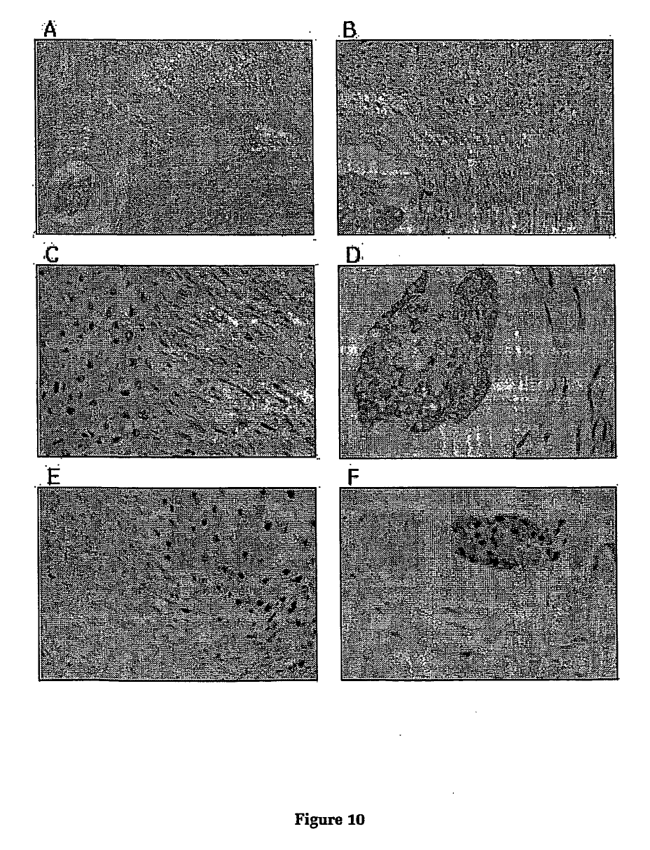

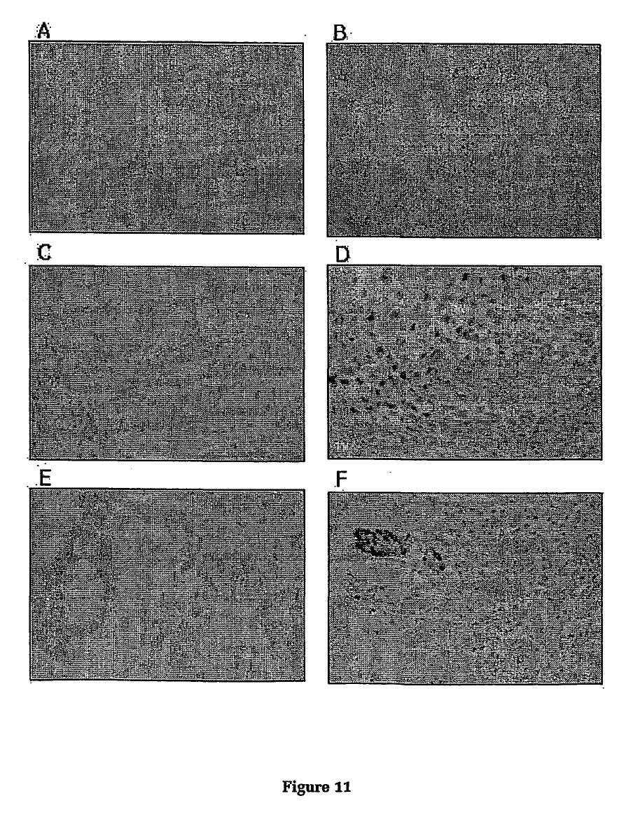

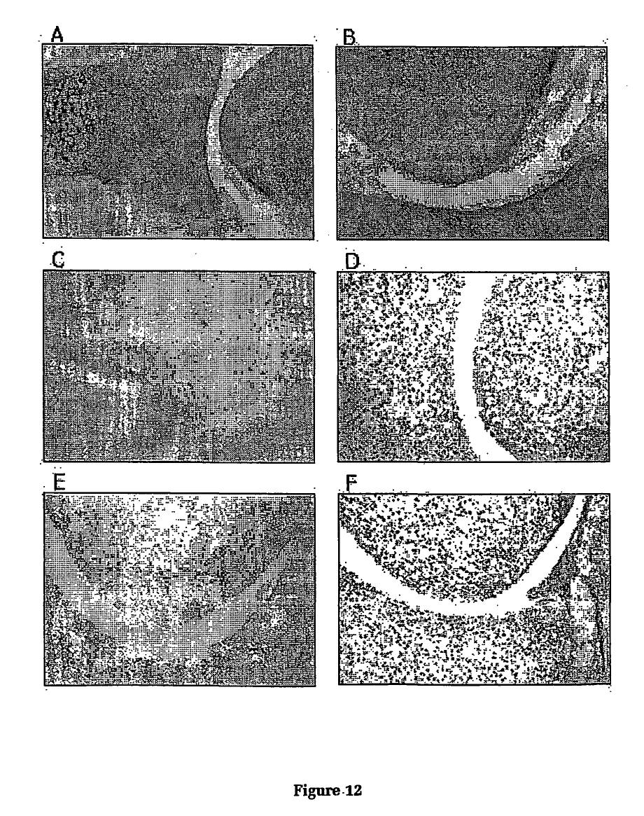

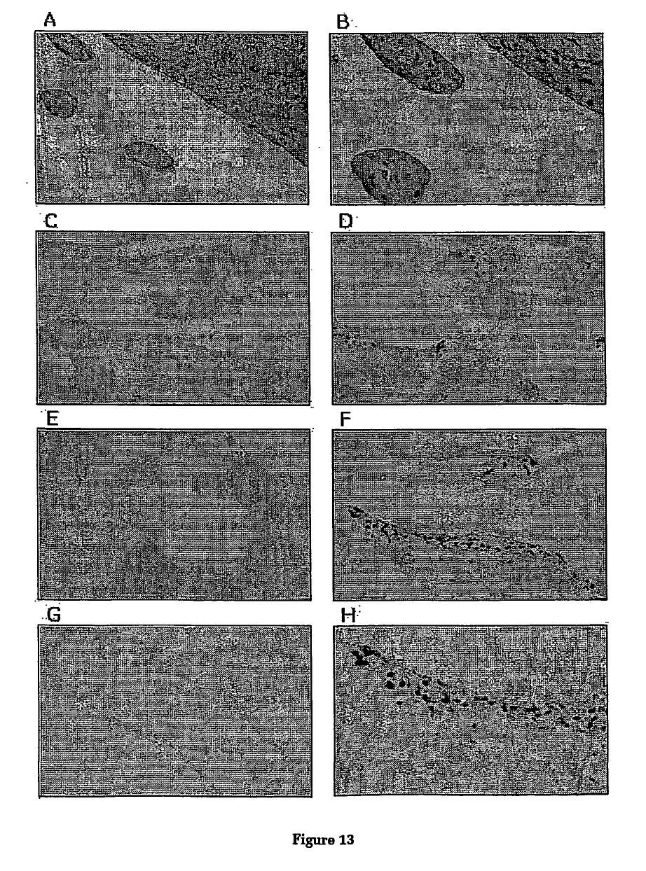

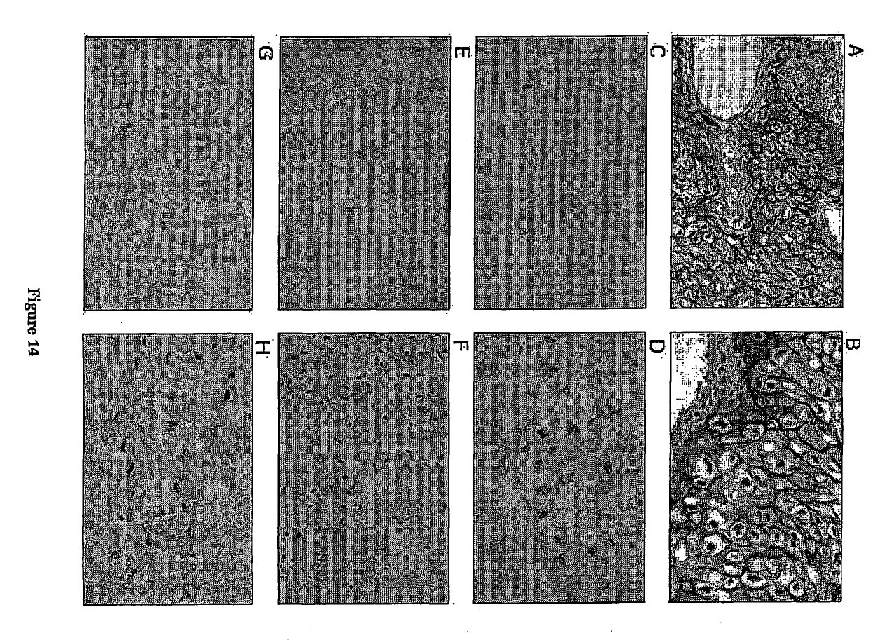

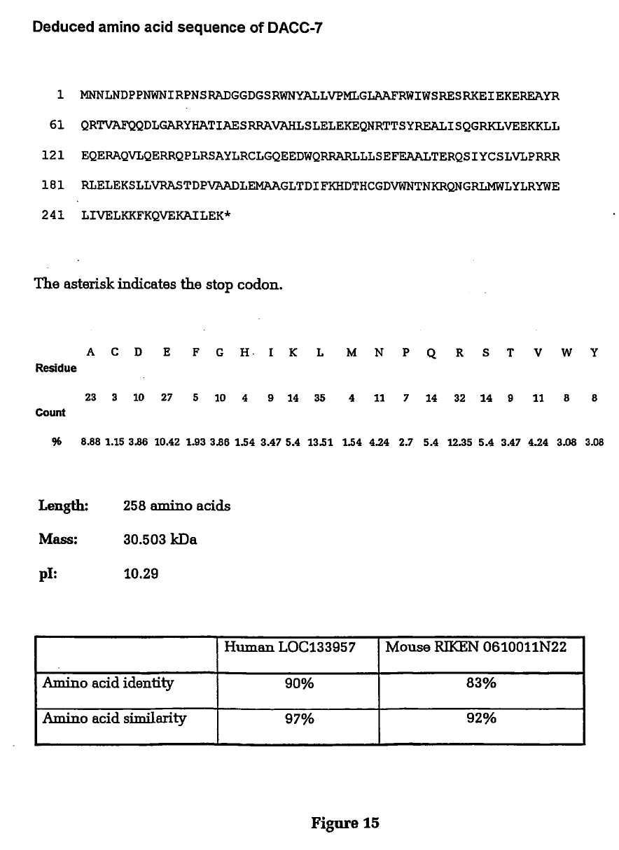

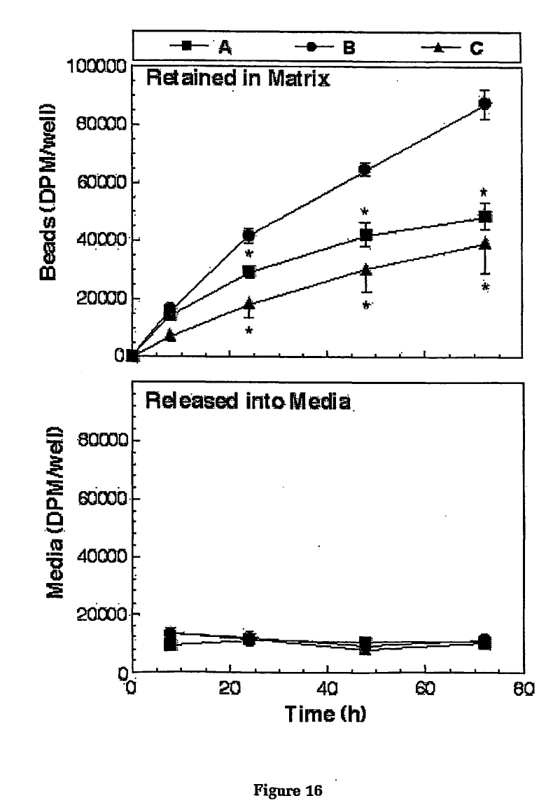

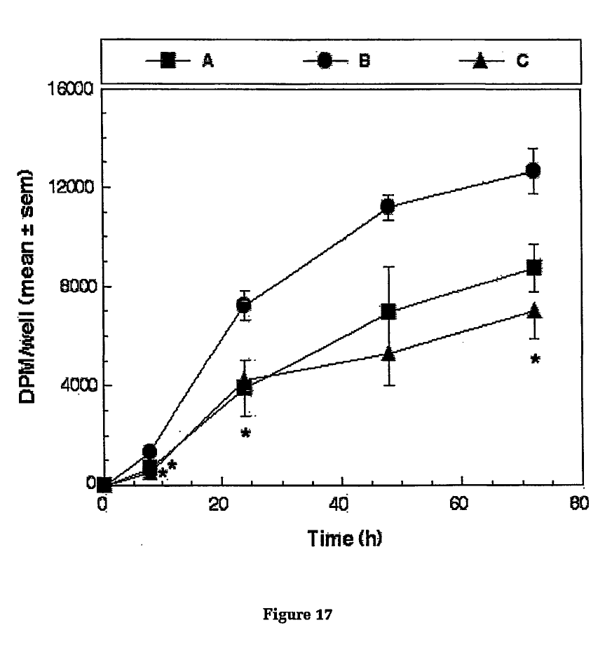

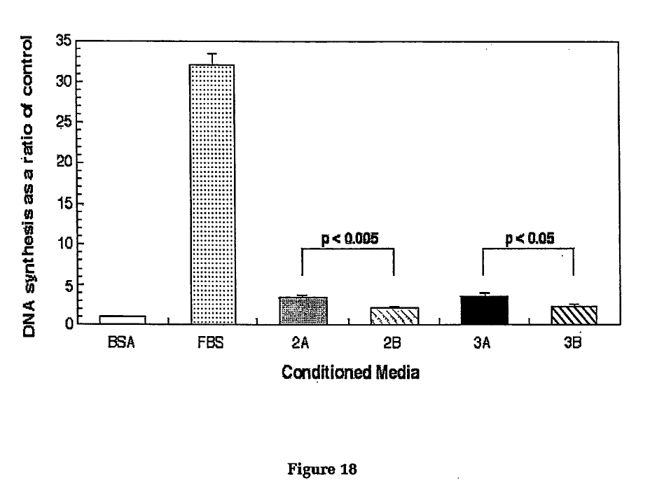

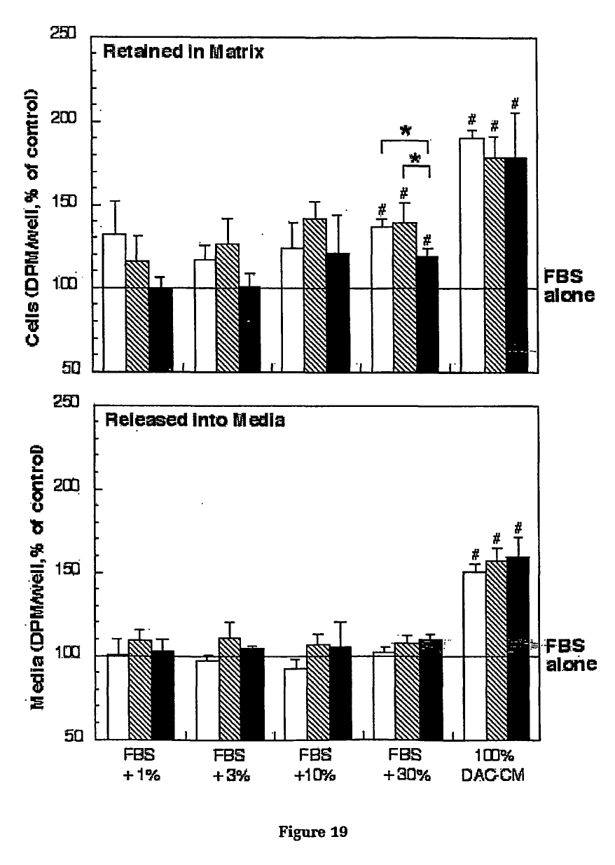

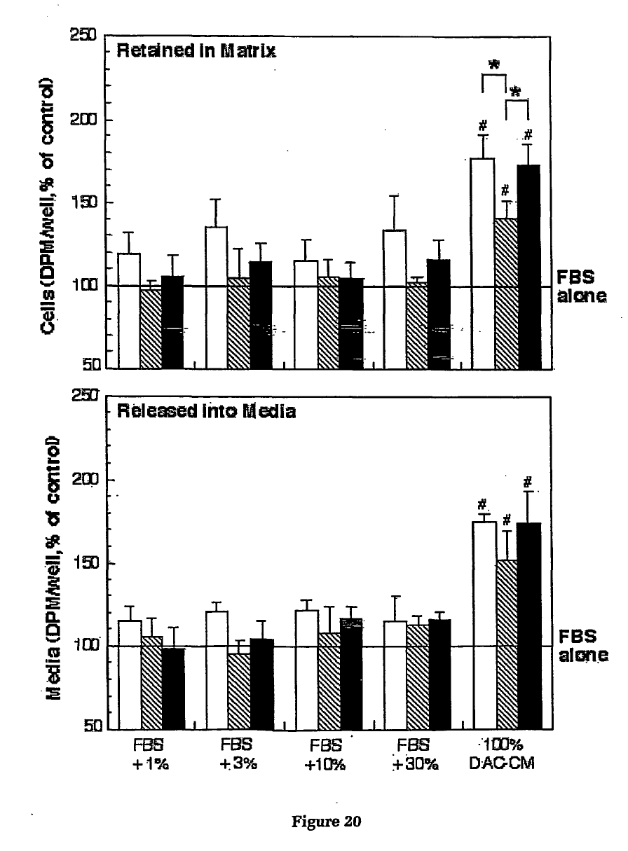

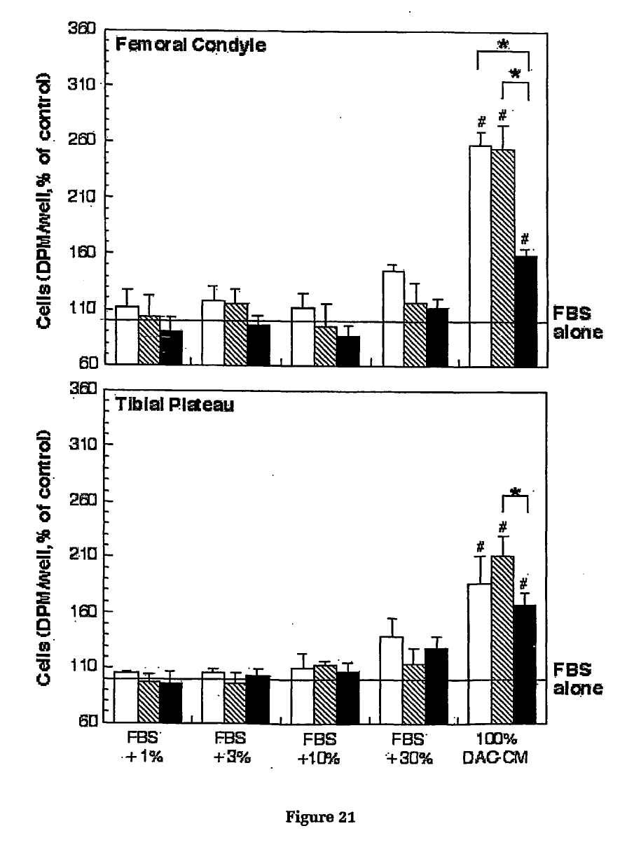

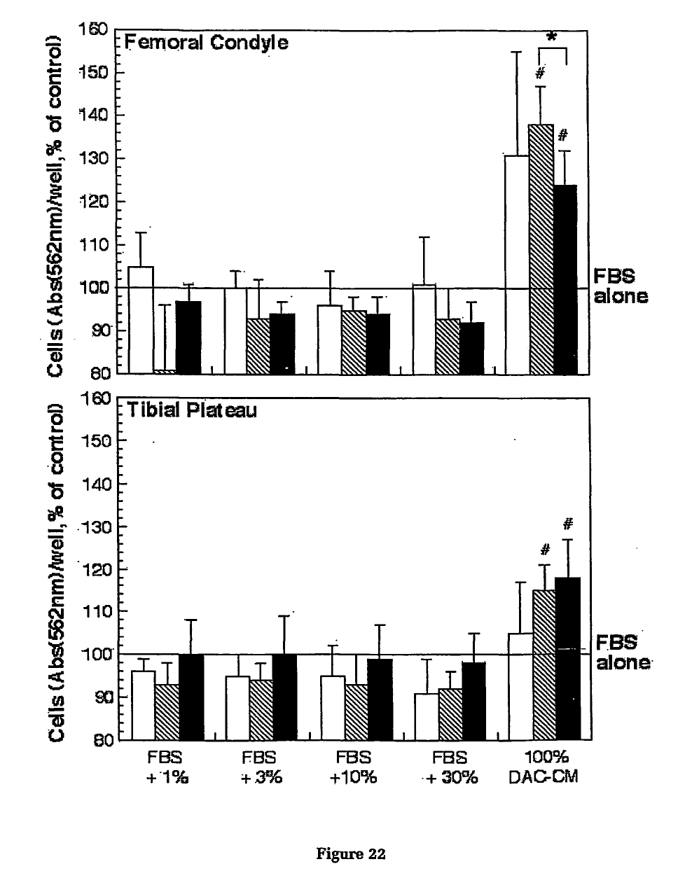

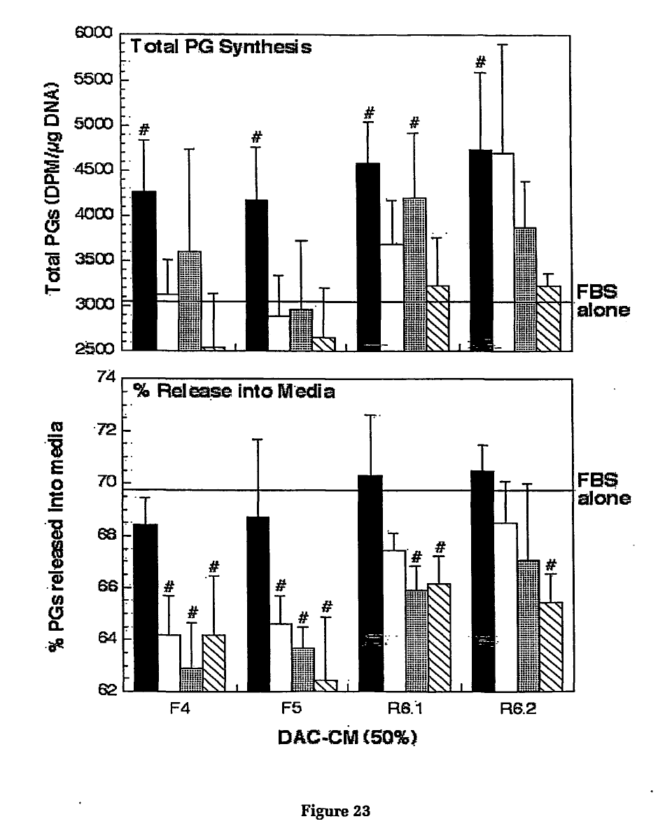

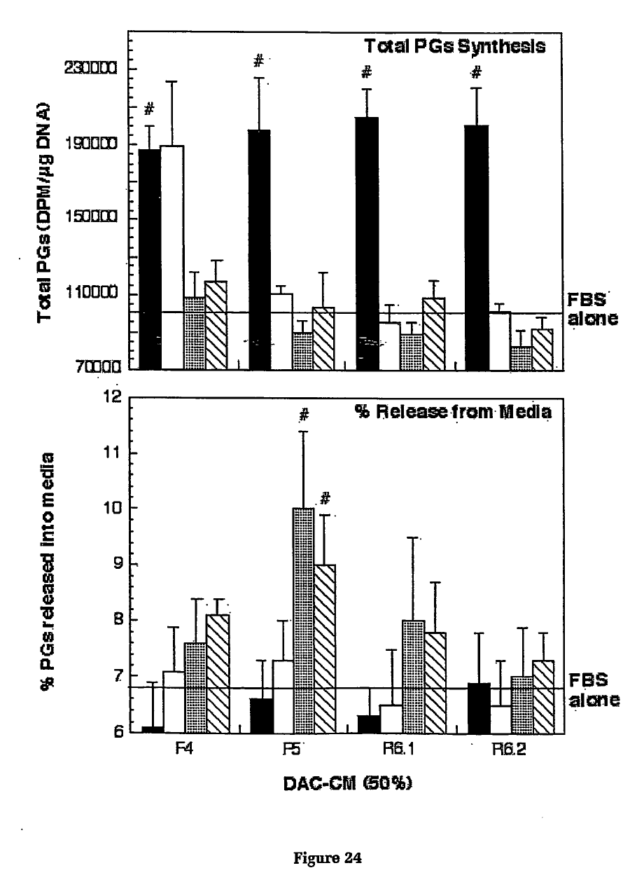

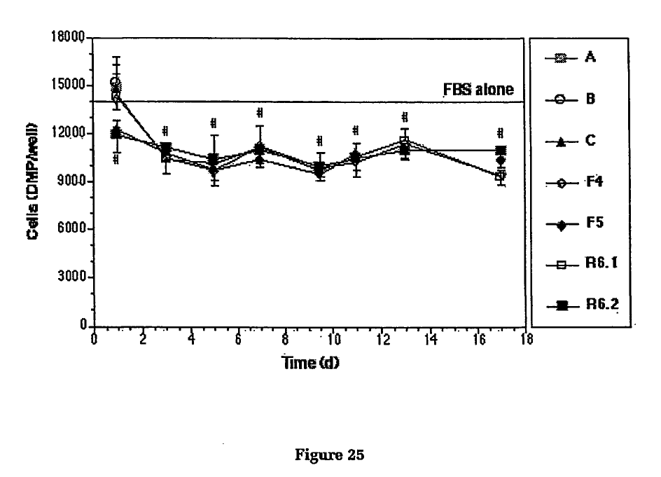

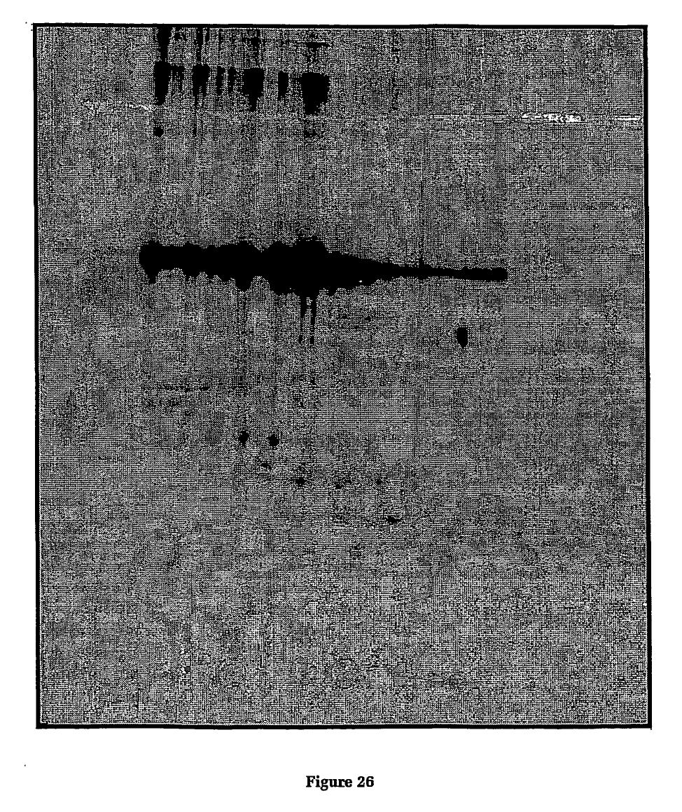

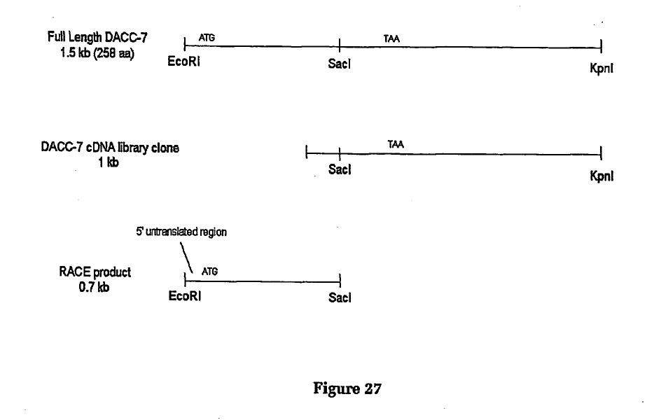

A lambda phage library containing clones ligated via EcoRI and XhoI ends in lambda Uni-ZAP XR (as described previously) was made from deer antler cartilage (DAC). This DAC cDNA library was screened for highly expressed cDNAs. Starting probe material for screening the deer antler cartilage cDNA library was generated-by creating a hybrid cDNA template consisting of pBluescriptSK and a partial collagen sequence (HC22). This cDNA template was using to make a ³²P radiolabelled RNA probe. The library was screened using this probe as described previously. After primary screening, 15 clones were selected as positive by identification of corresponding radioactive dots on the phosphorimager. After secondary screening, 14 out of the original 15 remained positive. Tertiary screening confirmed that all 14 clones from secondary screening were positive, and single isolated positive phage clones was selected for in vivo excision to release the plasmid. Plasmid DNA was then obtained by miniprep. The miniprep DNA was digested with EcoRI and XhoI to release the inserts and run on a 1% agarose gel. The clones were found to range in size between 0.5 kb and 1.5 kb. These were sequenced for further identification using universal forward (5′ sequencing primer) and reverse (3′ sequencing primer) primers. Sequence homology analysis revealed that the 14 clones could be grouped into 9 clusters (as shown in FIG. 5), representing homology with Human α1 type II collagen, Human prepro-alpha1 (I) collagen, Human procollagen alpha 2(V), Human KIAA1075 protein (tensin2), Human SPARC/osteonectin, Human ribosomal protein S2 (RPS2), Human ribosomal protein L23a, Human non-histone chromosomal protein (HMG-14), and Human LOC133957 protein of unknown function (Genbank BC015349).

Because the SOLR E. coli host yielded low quantities of plasmid DNA, DAC clones were transformed into E. coli DH5α host, which was a better host for plasmid DNA production. The 14 clones were fully sequenced (except for DACC-9) by primer walking and open reading frames were found.

DACC-7

Sequence alignments with human LOC133957 and mouse RIKEN 0610011N22 (Genbank BC003345, of unknown function) showed that the DACC-7 clone was very unlikely to be full length based on comparison of size and sequence.

Using the technique of 5′ RACE as described previously, the 5′ end of DACC-7 was obtained. RNA was made from deer antler cartilage tissue and checked for integrity on a denaturing agarose gel. A 5′ RACE kit (CLONTECH) was used on deer antler cartilage RNA (as described previously) in attempt to obtain 5′ DACC-7. The primers used for 5′ RACE were made based on sequence information from the 5′ end of the DACC-7 deer antler cartilage library clone as shown in FIG. 5. After the first round of PCR amplification, only a faint band could be seen in each lane. Gel purification and a second round of PCR were necessary to see clearer bands. The sizes of these broad bands were approximately 0.6 kb but were difficult to determine at this stage. After further gel purification, the PCR products were cloned into pGEM T-Easy® vector (Promega) as described in the methods and transformed into high efficiency competent E. coli cells JM109 (Promega). Plasmid DNA was isolated by miniprep, digested with EcoRI to release the insert and run on a 1% gel. One distinct insert was identified, approximately 0.7 kb in size. A midiprep plasmid preparation of this product was sequenced using universal forward (T7) and reverse (SP6) primers. The sequencing information showed that the 0.7 kb RACE product was determined to be 0.729 kb by sequencing. This overlapped 0.287 kb of the DACC7 deer antler cartilage library clone and extended the sequence 5′ by 0.442 kb, making DACC-7 gene product approximately 1.5 kb in length. The first methionine (ATG) start site that produced the longest open reading frame was 48 bp from the 5′ end of the DACC-7 gene product Thus a 5′ untranslated region of 47 bp was identified.

Generating a full length DACC-7 construct in a eukaryotic expression vector, such as pBK CMV, was necessary for future expression of the DACC-7 protein. Using suitably located single-cut restriction enzymes and a procedure as described in the methods (FIG. 27), the 0.7 kb RACE product and the library clone were joined together to generate a putative DACC-7 cDNA of 1.5 kb (including 5′ untranslated region) with an open reading frame of 258 aa. Directional cloning using non-complementary sticky ends ensured correct orientation of each insert.

A contig of the sequences of the 0.7 kb RACE product and the 1 kb DACC-7 library clone was analysed for amino acid sequence homology with human (LOC133957) and mouse (RIKEN 0610011N22) homologs (FIG. 15). Comparison of the sizes and sequences of DACC-7 with human (LOC133957) ortholog strongly suggest that the DACC-7 contig is full length. A website called NCBI Entrez Genome map view (http://www.ncbi.nlm.nih.gov/cgi-bin/Entrez/maps), which provides information on gene clusters localised to the human genome, has a chromosomal localisation of the human LOC133957gene-Chr.5,gi|17444086: 171999-185824. This location corresponds to the region 5p15.33. The 1.5 kb full length DACC-7 cDNA contains an open reading frame of 0.777 kb (258 aa) that is shorter than the human (LOC133957, 0.783 kb, 260 aa) or mouse (RIKEN 0610011N22, 0.783 kb, 260 aa) homologs, with 2 deleted amino acids at the 3′ end (131aa and 132aa). Comparison of the DACC-7 open reading frame with human (LOC133957) and mouse (RIKEN 0610011N22) homolog sequence has shown that the DACC-7 sequence obtained is very likely to be full length. As shown in FIG. 15, there is a reasonably high homology of DACC-7 with human LOC133957 and mouse RIKEN 061001N22, demonstrating that these are species homologs of DACC-7.

Examination of the DACC7 amino acid sequence revealed that DACC-7 sequence had potential a N-glycosylation site (N—X—S or N—X-T where X is any amino acid except proline) at 98aa . . . 100aa. Based on the amino acid usage (FIG. 15), the polypeptide backbone of the DACC-7 protein was predicted to be 30 kDa. The presence of a N-glycosylation site suggests the size of DACC-7 protein to be larger in vivo. A signal peptide was detected by SMART database (identifies domains, http://smart.embl-heidelberg.de/) at 1aa . . . 46aa and is thus likely to be a secreted protein, directed out of the cell. The DACC-7 protein was determined to be a basic protein from the pI value (FIG. 15). Thus DACC-7 protein could potentially bind to proteoglycans, a major constituent of the extracellular matrix (a negatively charged environment).

DACC-2

The polypeptide sequence encoded by this cDNA sequence shares up to 98% sequence identity with known vertebrate collagen alpha 1(II) chain precursors which includes human (Su et al. 1989: Accession No. PO₂₄₅₈) and mouse sequences (Metsaranta et al., 1991: Accession No. B41182). Type II collagen fibrils are known as a major structural protein forming extracellular matrix structures of connective tissues, such as cartilage, nucleus pulposus and vitreous body. It maintain the shape and to resist the deformation of the tissues.

The most closely relate gene family to vertebrate collagen alpha 1(II) chain precursors are Type I collagen which are approximately 68% identical the polypeptide sequence encoded by DACC-2.

DACC-3

The polypeptide sequence encoded by this cDNA sequence shares up to 98% sequence identity with known 40S ribosomal protein S2(S4) (LLREP3 protein) which includes human (Slynn et al. 1990: Accession No; P15880) and mouse sequences (Heller et al. 1988: Accession No. P25444). RPS2 is known to function as both a ribosomal protein (component of the 40S subunit) for mRNA binding and is required during oogenesis (as demonstrated by a sterile female RPS2 mutant fly model).

The most closely relate gene family to vertebrate 40S ribosomal protein S2(S4) is the human ortholog of the mouse wisZ protein which is approximately 76% identical the polypeptide sequence encoded by DACC-3.

DACC-4

The polypeptide sequence encoded by this cDNA sequence shares up to 100% sequence identity with known ribosomal protein L23a which includes human (Wool et al. 1995: Accession No. NP_{—}000975) and rat sequences (Suzuki and Wool, 1993: Accession No. CAA46336). L23a is a ribosomal protein that is a component of the 60S subunit The protein may be one of the target molecules involved in mediating growth inhibition by interferon.

The most closely relate gene family to vertebrate ribosomal protein L23a is the 60S ribosomal protein which is approximately 83% identical the polypeptide sequence encoded by DACC-4.

DACC-5

The polypeptide sequence encoded by this cDNA sequence shares up to 81% sequence identity with known human high-mobility group (non-histone chromosomal) protein 14 (Accession No. XP_{—}049753). HMG-14 which binds to the inner side of the nucleosomal DNA, potentially altering the interaction between the DNA and the histone octamer. Like HMG-14, it may be involved in the process that maintains transcribable genes in a unique chromatin conformation.

DACC-6

The polypeptide sequence encoded by this cDNA sequence shares up to 98% sequence identity with tensin2 (Accession No. XP_{—}029631). Tensin2 positively regulates cell migration. The tensin family role is in regulating cell motility.

The most closely relate gene family to this protein is tensin which is approximately 65% identical the polypeptide sequence encoded by DACC4.

DACC-8

DACC-8 appears to be non-coding, however, shares a high degree of sequence identity the mRNA encoding osteonectin (Lankat-Buttgereit et al., 1988). Osteonectin appears to regulate cell growth through interactions with the extracellular matrix and cytokines. Osteonectin binds calcium and copper, several types of collagen, albumin, thrombospondin, PDGF and cell membranes. Osteonectin is expressed at high levels in tissues undergoing morphogenesis, remodelling and wound repair.

The most closely relate gene family to vertebrate osteonectin is the human SPARC-like 1 protein which is approximately 57% identical to human osteonectin.

DACC-9

Two sequences were obtained, one to the 5′ end and another to the 3′ end. The polypeptide sequence encoded by the 5′ end cDNA sequence shares up to 90% sequence identity with known heat-shock 20 kD like-protein P20 which includes human (XP_{—}059039) and rat sequences (Inaguma et al. 1996: Accession No. P97541). HEAT-SHOCK 20 KD LIKE-PROTEIN P20 (belongs to the small heat shock protein (HSP20) family) which is related to stress proteins.

The most closely relate gene family to vertebrate heat-shock 20 kD like-protein P20 is the crystallin proteins which are approximately 46% identical the polypeptide sequence encoded by DACC-9.

DACC-10

The polypeptide sequence encoded by this cDNA sequence shares up to 95% sequence identity with known alpha 2 type V collagen preproproteins which includes human (Myers et al. 1985: Accession No. NP 000384) and mouse sequences (Andrikopoulos et al. 1992: Accession No. NP_{—}031763). Collagen alpha 2 type V is a subunit of type V collagen trimers. It is a minor connective tissue component which binds to DNA, Heparan sulphate, thrombospondin, heparin, and insulin. It is suggested to play an important role in collagen fibrillogenesis.

The most closely relate gene family to vertebrate alpha 2 V type collagen preproproteins is alpha 1 type II collagen which is approximately 62% identical the polypeptide sequence encoded by DACC-10.

DACC-11

The polypeptide sequence encoded by this cDNA sequence shares up to 97% sequence identity with known pro alpha 1(I) collagen which includes human (Chu et al. 1985: Accession No. AAB94054) and mouse sequences (Li et al. 1995: Accession No. P11087). Collagen alpha 1 type I is a subunit of type I collagen. It forms the fibrils of skin, tendon, ligaments and bones, giving strength to connective tissues.

The most closely relate gene family to vertebrate pro alpha 1(I) collagen is alpha 1 type II collagen which is approximately 70% identical the polypeptide sequence encoded by DACC-11.

Expression of DACC-7

One aspect of the present invention provides a method of identifying and/or characterising the developmental position of mesenchymal cells, particularly during embryogenesis, the method comprising exposing a test sample including mesenchymal cell mRNA to a suitably-labelled nucleic acid probe with specifically hybridizes to a polynucleotide of the present invention and detecting hybridisation of said probe to said mRNA. Preferably, the test sample is a suitably prepared histological section.

One example of a method according to this aspect comprises the use of a 1.5 kb RNA probe prepared from clone DACC-7 according to standard techniques to identify chondrocytes and notochordal cells in active states of growth and differentiation. FIGS. 7-9 show histological sections of 12-14-week-old human foetal knee joints and spines subjected to in-situ hybridisation using the DACC-7 derived RNA probe illustrating strong expression by chondrocytes in growing cartilage. Similar studies with the DACC-7 probe using histological sections of human foetal spinal columns have demonstrated that notochordal cells and chondrocytes in the nucleus pulposus of the foetal disc also strongly express the gene product but fibrochondrocytes of the disc annulus fibrosis were less active (FIGS. 10 and 11). These observation was complemented by in-situ hybridisation using the same histological sections but a type II collagen RNA probe where uniform staining of chondrocytes and weaker staining for fibrochondrocytes of the annulus fibrosis was noted (FIGS. 10 and 11). A comparison of the intensity of cellular staining of histological sections made from joints of 12-(FIG. 9) and 14-week-old (FIG. 12) human foetuses with the DACC-7 derived RNA probe suggested that the expression of this gene product was similar in both age groups. In addition to the chondrocytes of growing foetal cartilage it was also demonstrated that the DACC-7 riboprobe was able to identify chondrocytes located in fibrillated cartilage from human osteoarthritic joints which were involved in attempted repair and regeneration of the extracellular matrix. These cells exhibited enhanced expression of DACC-7 as well as type II collagen, the chondrocyte phenotype protein, as illustrated by the sections shown in FIG. 13. In contrast, it was found that the resting chondrocytes present in normal young ovine cartilages of the medial and lateral tibial plateaux failed to exhibit staining for the presence of DACC-7 expression confirming that DACC-7 expression is a marker for cell undergoing proliferation during the active phases of cartilage growth and repair.

As would be expected from the origin of the gene probe, chondrocytes and particularly hypertrophic chondrocytes in the cartilaginous region of the growing deer antler also showed strong expression of DACC-7 and type II collagen gene expression by these same cells (FIG. 14).

Growth Promoting Factors in Conditioned Media Obtained From Deer Antler Cartilage Cells

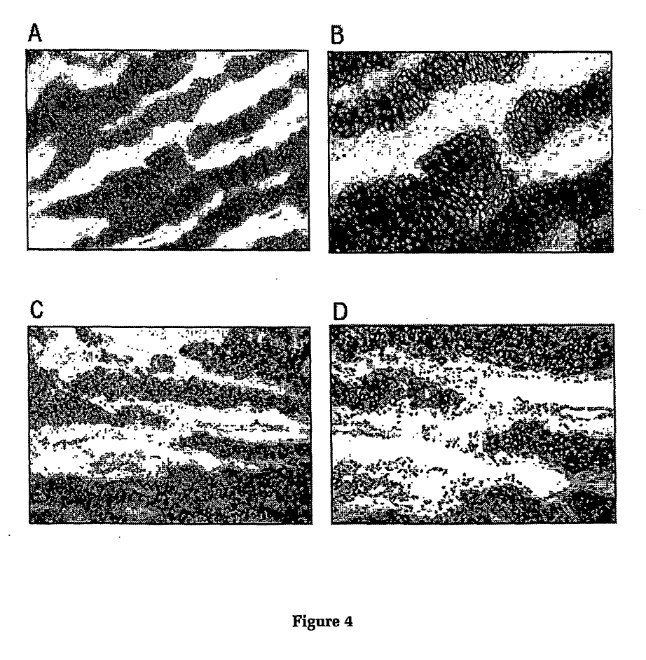

Histological examination of the tissue sections obtained from DAC regions A, B, C (FIG. 3) showed negligible staining for PGs in region A using Toluidine Blue but strong staining for PGs in sections from zones B and C (FIG. 4). Blood vessels were present in each zone but the cell morphology evident in sections from zones B and C were of typical chondrocytes, zone A cells appeared more fibroblastic in appearance corresponding to pre-chondroblasts as described by Frasier et al. (1975). Sections of DAC from the proximate end of C zone showed the presence of hypertrophic chondrocytes accompanied by early mineralisation and increased vascular invasion (FIG. 4).

DAC cells in alginate beads exhibited high incorporation of ³⁵S into PGs. Zone B, a region which is composed of mature chondrocyte-like cells and abundant cartilage matrix, showed statistically higher rates of PG synthesis than cells from zones A and C (p<0.05) (FIG. 16). Over the 72 h incubation period negligible amounts of ³⁵S-PGs were released into the media (FIG. 16) confirming that minimal proteolytic modification of PGs were occurring in this culture system. Furthermore, studies of the mRNA obtained from these DAC cells using Northern blot analysis and human aggrecan cRNA riboprobe confirmed that DAC cells maintained their phenotypic expression during these experiments (data not shown).

Cells from DAC zone B were also shown by their incorporation of ³H-thymidine into DNA to be the more proliferative than cells from the other two zones (p<0.05) (FIG. 17) when they were cultured in the presence of 10% foetal bovine serum (FBS). Conditioned media (CM) collected from the alginate bead cultures of DAC cells when added to cultures of ovine articular chondrocytes in the absence of FBS induced a small stimulation of mitosis (FIG. 18). However, it was noteworthy that CM from cells from zone A, the prechondroblast zone, was more potent than from zone B (p<0.05) (FIG. 18). Under the same conditions DNA synthesis in SAC cultured in the presence of 10% FBS was increased by 30-35% (FIG. 18).

It was found that foetal bovine serum could act synergistically with the growth factors produced by DAC cells since it augmented cell mitosis and synthesis of PGs. This was illustrated by the data shown in FIG. 19 where it can be seen that replacing the FBS with FBS supplemented with 30% or 100% DAC-CM substantially increased ³⁵S-PG synthesis by ovine femoral chondrocytes. The amounts of PGs synthesised by using 100% DAC-CM which also contained 10% FBS was almost double that produced by 10% FBS alone (FIG. 19). Condition media collected from cultures of regions A and B were shown to be more effective than from region C (FIG. 19). Although similar profiles were obtained for cultures of chondrocytes obtained from the ovine tibial plateau (FIG. 20), the cells from this joint region were found to be less responsive to DAC-CM than the femoral-chondrocytes (FIG. 20).

The ability of DAC-CM to stimulate ³⁵S-PG synthesis by ovine chondrocytes in the presence of FBS was also reflected in enhanced mitotic activity. As shown in FIG. 21, DNA synthesis was more than doubled when either femoral or tibial SAC were cultured with 100% DAC-CM. Again cells from zones A and B produced higher amounts of growth factors than from DAC zone C (p<0.05) and femoral sheep chondrocytes were more responsive to these factors than tibial chondrocytes.

The enhanced metabolic activity of SAC in the presence of DAC-CM was also reflected in increased mitochondrial activity using the MIT assay (FIG. 22).

In all the previous experiments the conditioned media used was collected from the DAC cells maintained in culture for 24 h. In order to determine how long the growth factor(s) were elaborated by DAC cells CM was collected 1, 3, 5 and 7 days post-monolayer culture initiation. As is evident from FIG. 23 the stimulatory effect of CM on ³⁵S-PG synthesis was more pronounced when collected from DAC cultures in the first 1-2 days irrespective of their origin.

A similar outcome was obtained using rabbit ear cartilage explant cultures as the target tissue but, as would be expected, far less ³⁵S-PGs were released into the media than in monolayer culture, the majority of ³⁵S-PGs being retained in the matrix (FIG. 24).

The selective effects of the stimulatory factors produced by DAC cells in culture on chondrocytes were illustrated in the experiments using fibroblasts as target cells.

As is evident from FIG. 25 addition of CM from DAC alginate bead cultures or DAC monolayer cultures to 3T3 fibroblast cultures failed to stimulate but instead suppressed mitosis (relative to effects of FBS alone) as determined by the decreased incorporation of ³H-thymidine into DNA by these cells.

The present studies have shown that DAC cells can release soluble factor(s) into culture media which can stimulate both DNA and PG synthesis by chondrocytes in monolayer or explant culture. This stimulatory effect was greatly enhanced when the media containing these factor(s) was supplemented with FBS which is known to contain a complex cocktail of growth factors, such as IGFs, basic and acidic FGFs, TGF-β, as well as proteinase inhibitors and hormones.

The selectivity of the DAC derived factor(s) for chondrocytes and the amplification of its stimulatory effects in the presence of FBS suggests that their physiological role in the growing antler tip may be to direct and augment the multitude of blood borne growth factors which diffusing into the tissues during the very active growth period.

Two-Dimensional gel electrophoresis sample images were obtained in triplicate for each of the 3 samples, for the pH gradients 3-6,5-8 and 6-11. The 3 samples were derived from F4-24 h, F4-168 h and serum-free culture supernatants. Each image was cropped and grouped together as a triplicate set of images. The 3 gels in each set were used to create a raw master reference gel that acted as a composite. This composite image was then used for comparative purposes in identifying protein spot differences between culture conditions. Regions of interest were then selected from the composite images that demonstrated differential display between F4-24 h and F4-168 h culture supernatant samples. The differential display regions highlighted for each pH range showed that gels with 5-8 provided the best separation of proteins from the deer antler chondrocyte culture supernatant samples studied using Two-Dimensional Electrophoresis. Using this system changes in protein expression profile were observed between F4-24 h and F4-168 h culture supernatant samples, indicating that protein expression differed over the time course studied. Regions exhibiting differential display were selected, with differences in protein expression highlighted. A number of proteins present in the 24 h sample but absent in the 168 h sample are evident and were annotated. All were present at low levels but subjected to MS analysis.

Positive identification was only achieved for the proteins circled in FIG. 26. MS analysis of the tryptic digests of these proteins revealed the Peptide A N-terminal amino acid sequence of FVEGL/IYQ/KVEL/IDTK (SEQ ID NO: 41) and Peptide B N-terminal amino acid sequence of EGL/IYQ/KV (SEQ ID NO: 42). By this method, leucine and isoleucine (L/I) are not distinguishable, nor are glutamine and lysine (Q/K), differing in mass by only 0.04 Da. From the protein databases available both these proteins were identified as the protein transthyretin.

Transthyretin is a thyroid hormone-binding protein which forms tight protein-protein complex with the retinol-binding protein (RBP). The formation of the complex with RBP stabilises the binding of retinol to RBP. The term refers to the fact that it is a transport protein for both thyroxine and retinol (vitamin A). Transthyretin is also one of the precursor proteins commonly found in amyloid deposits (transthyretin-associated amyloidosis disease).

The finding that one of the proteins expressed by the-DAC derived factor(s) during the early stages but not latter stages of culture was transthyretin was consistent with the observed stimulatory effect of these supernatants. The effect probably being mediated by the ability of transthyretin to carry thyroxine in complex with retinol into the cell and thus promote the proliferation of cartilage and its subsequent conversion the bone. This is the first report of the production of transthyretin by chondrocytes but is consistent with the known role of this protein in the growth and development of other mesenchymal tissues (Sakabe et al. 1999; Barron et al. 1998; Hamazaid et al. 2001).

The present inventors have shown that the expression of the mRNA for the type II procollagen and proteoglycans can be upregulated in cultures of human and ovine chondrocytes by genes derived from deer antler chondrocytes. This response can be modified by concomitant exposure of these cells to a variety of hormones and endocrine growth factors including: insulin-like growth factor (IGF-1), TGF-beta, FGFs, VEGFs, morphogenic bone factors, thyroid hormones (thyroxine), parathyroid hormone related protein (PTHrP), sex hormones, luteinizing hormone (LH) and prolactin and even conditioned medium obtained by culturing the deer antler chondrocytes themselves. One or a combination of these hormones and/or growth factors may be used to increase the rate of proliferation and thus number of DACC gene transfected chondrocytes obtained from the original biopsy thereby providing sufficient numbers of cells for implantation into connective tissue defects or to stored cryogenically for transplantation at a later date. One of the proteins identified in the supernatants obtained from the deer antler cell cultures which produce these stimulatory activity to chondrocytes was transthyretin, a thyroid hormone-binding protein and forms complexes with retinol-binding protein, known to be involved in embryonic development (Sakabe et al. 1999; Barron et al. 1998; Ingenbleek and Bernstein, 1999; Stark et al. 2001; Hamazaki et al. 2001; Varga and Vajtai, 1998).

The present results identify a method of improving mesenchymal cell growth, repair, regeneration or restoration of cartilage, tendon, meniscal and disc defects which would restore their function and decrease the rate of development of OA in the joint. This procedure would require either surgically obtaining a small biopsy of cartilage adjacent to the defect, or from within the target disc, isolating the chondrocytes from these biopsies, establishing them in culture and transfecting them with a genE(s) which the present inventors have identified in the rapidly growing cartilage cells of deer antler and replacing the transfected chondrocytes back into the defect using a suitable carrier, or artificial matrix, to maintain them in place. Another procedure would require transfecting cartilage adjacent to the defect, or from within the target disc, in vivo as described previously and in detail by Goomer et al. (2000). These modified chondrocytes in response to the normal mechanical and nutritional factors acting on the disc and cartilage plug in vivo would stimulate the transformed cells to proliferate and synthesise a new matrix capable of repairing the defect.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

### REFERENCES

Andrikopoulos, K., Suzuki, H. R., Solursh, M and Ramirez, F. (1992) Dev. Dyn. 195:113-120.

Banks and Newbrey, (1983) Antler Development in Cervidae, Banks, R. D. (Ed) Kingsville Texas Cesar Kleburg Wildlife Research Institute, 279-306.

Barnett, M. L., Combitchi, D. and Trentham, D. E. (1996) Arthritis. Rheum. 39:623-628.

Barron, M., McAllister, D., Smith, S. M. and Lough, J. (1998) Dev. Dyn. 212:413-422.

Breinan, H. A., Minas, T., Barone, L., Tubo, R, Hsu, H.-P., Shortkroff, S., Nehrer, S., Sledge, C. B., and Spector, M. (1998) Tissue Engineering. 4:101-114.

Brittberg, M., Lindahl, A., Nilsson, A., Ohlsson, C., Isaksson, O. and Peterson, L. (1994) New Engl. J. Med. 33:889-895.

Buckwalter et al. (1987) Am. Acad, Orthop. Surg. pp 456482.

Buckwalter. J. A. and Mankin. H. J. (1998) Arthritis Rheum. 41:1331-1342.

Cancedda, R., Descalzi,-Cancedda, F. and Castagnola, P. (1995) Int. Rev. Cytology 159:265-358.

Chu, M. L., de Wet, W., Bernard, M. and Ramirez, F. (1985) J. Biol. Chem. 260:2315-2320.

Collier, S. and Ghosh, P. (1989) Ann. Rheum. Dis. 48:372-381.

Cremer, M. A., Terato, K., Watson, W. C., Griffiths, L, Townes, A. S. and Kang, A. H. (1992) J. Immunol. 149:1045-1053.

Dougherty, W. G. and Parks, T. D. (1995) Curr. Opin. Cell Biol. 7:399-405.

Dunbrack, R. L, Jr, Gerloff, D. L., Bower, M., Chen, X., Lichtarge, O. and Cohen, F. E. (1997). Folding and Design, 2:R27-42.

Fernandes, J. C., Martel-Pelletier, J. and Pelletier, J.-P. (2000) Clin. Orthop. and Rel. Res. 379(S):S262-S272.

Frasier, M, B, Banks, W, J, and Newbrey, J. W. (1975) Calcified Tissue Res. 17:273-288.

Goomer, R S., Maris, T. M., Gelberman, R. G., Boyer, M., Silva, M. and Amiel, D. (2000) Clin. Orthop. and Rel. Res. 379S:S189-S200.

Goss, R. L. (1983) Deer Antlers Regeneration, Evolution and Function, NY Acad. Press.

Hamazaki, T. Iiboshi, Y., Oka, M., Papst, P. S., Meacham, A. M., Zon, L. I. and Terada, N. (2001) FEBS Letters 497:15-19.

Hart, B. A., Bakker, N. P. M., Jonker, M. and Bontrop, R. E. (1993). Eur. J. Immunol. 23:251-264.

Haseloff, J. and Gerlach, W. L. (1988) Nature 334, 585-591.

Hashimoto, S., Ochs, R. J., Komiya, S. and Lotz, M. (1998) Arthritis and Rheum. 41:1632-1638.

Hauselmann, H. J., Oppliger, L., Michel, B. A., Stefanovic-Racic, M., and Evans, C. H. (1994) FEBS Lett 352:361-364.

Heller, D. L., Gianola, K. M. and Leinward, L. A. (1988) Mol. Cell. Biol. 8:2797-2803.

Hutadilok, N., Smith, M. M., and Ghosh, P. (1991) Ann. Rheum Dis. 50:219-26.

Inaguma, Y., Hasegawa, K., Kato, K. and Nishida, Y. (1996) Gene 178:145-150.

Ingenbleek, Y. and Bernstein L. (1999) Nutrition 15:305-320.

Kierdorf et al., (1995) Annals Anat 177:525-532.

Klagsburn, M., Langer, R., Levenson, R., Smith, S., and Lillehei, C. (1977) Exp. Cell Res. 105:99-108.

Lankat-Buttgereit, B., Mann, K., Deutzmann, R., Timpl, R1 and Krieg, T. (1998) FEBS Lett 236:352-356.

Li, S. W., Khillan, J. and Prockop, D. J. (1995) Matrix Biol. 14:593-595.

Little, C., Smith, S., Ghosh, P. and Bellenger, C. (1997) J. Rheumatol. 24:2199-209.

Loeser et al., (2000) Arthritis Rheum. 43:2110-2120.

Mason, J. M., Breitbart, A. S., Barcia, M., Porti, D., Pergolizzi, R. G. and Grande, D. A. (2000) Clin. Orthop. and Rel. Res. 379(S):S171-S178.

Metsaranta, M., Toman, D., de Crombrugghe, B. and Vuorio, E. (1991) J. Biol. Chem. 266:16862-16869.

Miller, A. (1990) Human Gene Therapy, 1:5-14.

Moon, S. H., Gilbertson, L. G., Nishida, K., Knaub, M., Muzzonigro, T., Robbins, P. D., Evans, C. H., Kang, J. D. (2000) Spine 25:2573-2579.

Mosmainn, T. (1983) J. Immunol. Methods. 65:55-63.

Muratoglu, S., Bachrati, C., Malpeli, M., Szabo, P., Neri, M., Dozin, B., Deak, F., Cancedda, R. and Kiss, I. (1995) Eur. J. Cell Biol. 68:411-418.

Myers, J. C., Loidl, H. R., Seyer, J. M. and Dion, A. S. (1985) J. Biol. Chem. 260:11216-11222.

Myers, L. K, Seyer, J. M. Stuart, J. M., Terato, K. David, C. S. and Kang, A. H. (1993) J. Immunol. 151:500-505.

Needleman, S. B. and Wunsch, C. D. (1970) J. Mol. Biol. 48:443-453.

Nehrer, S., Spector, M. and Minas, T. (1999) Clin. Orthop. and Rel. Res. 365:149-162.

Newbrey et al., (1983) Isolation of Collagens by Guanidinium-Extraction and Pepsin Digestion From Growing Deer Antler. In Antler Development in Cervidae, Banks R. D. (Ed) Kingsville Texas Cesar Kleburg Wildlife Research Institute, 307-316.

Nishida, K., Kang, J. D., Gilbertson, L. G., Moon, S.-H., Suh, J.-K., Vogt, M. T., Robbins, P. D. and Evans, C. H. (1999) Spine 24:2419-2425.

Nishida, Y., Knudson, C. B., Kuettner, K. E., and Knudson, W. (2000) Osteoarthritis Cartilage 8:127-136.

Nixon, A. J., Brower-Toland, B. D., Bent, S. J., Saxer, R. A., Wilke, M. J., Robbins, P. D. and Evans, C. H. (2000) Clin. Orthop. and Rel. Res. 379(S): S201-S213.

Perrimarni, R., Delves, A. and Gerlach, W. L. (1992) Gene 113:157-163.

Peterson, L. (1996) Acta Orthop. Belg. 62 Suppl. 1:196-200

Price et al., (1996) Developmental Dynamics 205: 332-347

Rahfoth, B., Weisser, J., Sternkopf, F., Aigner, T., von der Mark, K., Brauer, R. (1998) Osteoarthritis Cartilage. 6:50-65.

Sakabe, L, Paul, S., Mitsumoto, T., Kadoya, T. and Shinozawa, T. (1999) Endocrine J. 46:375-381.

Saldanha V. and Grande D. A. (2000) Biomaterials 21:2427-2431.

Sandell, L. J., Morris, N., Robbins, J. R. and Goldring, M. B. (1991) J. Cell Biol. 114:1307-1319.

Shippy, R., Lockner, R., Farnsworth, M. and Hampel, A. (1999) Mol. Biotech. 12:117-129.

Sieper, J., Kary, S., Sorensen, H., Alten, R., Eggens, U., Huge, W., Hiepe, F., Kuhne, A., Listing, J. and Ulbrich, N. (1996) Arthritis RheuLm. 39:41-51.

Slynn, G., Jenner, D., Potts, W., Elvin, P., Morten, J. E. and Markham, A. F. (1990) Nucl. Acids Res. 18:681.

Stark, M., Danielsson, O., Griffiths, W. J., Jornvall, H. and Johansson, J. (2001) J. Chromat B, Biomed. Sci. Appl. 754:357-367.

Su, M. W., Lee, B., Ramirez, F., Macado, M. and Horton, W. (1989) Nucl. Acids Res. 17:9473.

Suzuki, K. and Wool, I. G. (1993) J. Biol. Chem 268:2755-2761.

Trentham, D. E., Dynesius-Trentham, R. A., Orav, E. J., Combitchi, D., Lorenzo, C., Sewell, K. L., Hafler, D. A and Weiner H. L. (1993) Science 261:1727-1730.

Varga, Z. and Vajtai, I. (1998) Orvosi Hetilap. 139:761-765.

Wakitani, S., Goto, T., Young, R. G., Mansour, J. M, Goldberg, V. N, Caplan, A. I. (1998) Tissue Eng. 4:429-444.

Waterhouse, P. M., Graham, M. W. and Wang, M. B. (1998) Proc Natl Acad Sci USA 95:13959-13964.

Weiner, H. L., Friedman, A., Miller, A. Khoury, S. J., Sabbagh, A., Santos, L. Sayegh, M., Nussenblatt, R. B., Trentham, D. and Hafler, D. A. (1994) Ann. Rev. Immunol. 12:809-837.

Wool, I. G., Chan, Y. L. and Gluck, A. (1995) Biochem. Cell Biol. 73:933-947.

Zhang, Z. J., Lee, C. S. Y., Lider, O. and Weiner, H. L. (1990) J. Immunol. 145:2489-2493.

1. A method of stimulating cell growth and/or division, the method comprising contacting, or inserting into, an animal cell a polypeptide comprising a sequence selected from the group consisting of:
a) a sequence as shown in SEQ ID NO:1,
b) a sequence as shown in SEQ ID NO:2,
c) a sequence as shown in SEQ ID NO:3, and
d) a sequence which is at least 50% identical to any one of (a) to (c).

2. The method of claim 1, wherein the polypeptide is at least 80% identical to any one of (a) to (c).

3. The method of claim 1, wherein the polypeptide is at least 90% identical to any one of (a) to (c).

4. The method of claim 1, wherein the polypeptide is at least 95% identical to any one of (a) to (c).

5. A method of stimulating cell growth and/or division, the method comprising contacting, or inserting into, an animal cell a polypeptide comprising a sequence selected from the group consisting of:
a) a sequence as shown in SEQ ID NO:4,
b) a sequence as shown in SEQ ID NO:5,
c) a sequence as shown in SEQ ID NO:6, and
d) a sequence which is at least 70% identical to any one of (a) to (c).

6. A method of stimulating cell growth and/or division, the method comprising contacting, or inserting into, an animal cell a polypeptide comprising a sequence selected from the group consisting of:
a) a sequence as shown in SEQ ID NO:7,
b) a sequence as shown in SEQ ID NO:8,
c) a sequence as shown in SEQ ID NO:9, and
d) a sequence which is at least 80% identical to any one of (a) to (c).

7. A method of stimulating cell growth and/or division, the method comprising contacting, or inserting into, an animal cell a polypeptide comprising a sequence selected from the group consisting of:
a) a sequence as shown in SEQ ID NO:10,
b) a sequence as shown in SEQ ID NO:11,
c) a sequence as shown in SEQ ID NO:12, and
d) a sequence which is at least 85% identical to any one of (a) to (c).

8. A method of stimulating cell growth and/or division, the method comprising contacting, or inserting into, an animal cell a polypeptide comprising a sequence selected from the group consisting of:
a) a sequence as shown in SEQ ID NO:13,
b) a sequence as shown in SEQ ID NO:14, and
c) a sequence which is at least 70% identical to any one of (a) or (b).

9. A method of stimulating cell growth and/or division, the-method comprising contacting, or inserting into, an animal cell a polypeptide comprising a sequence selected from the group consisting of:
a) a sequence as shown in SEQ ID NO:15,
b) a sequence as shown in SEQ ID NO:16, and
c) a sequence which is at least 50% identical to any one of (a) or (b).

10. A method of stimulating cell growth and/or division, the method comprising contacting, or inserting into, an animal cell a polypeptide comprising a sequence selected from the group consisting of:
a) a sequence as shown in SEQ ID NO:17, and
b) a sequence which is at least 60% identical to a).

11. A method of stimulating cell growth and/or division, the method comprising contacting, or inserting into, an animal cell a polypeptide comprising a sequence selected from the group consisting of:
a) a sequence as shown in SEQ ID NO:18,
b) a sequence as shown in SEQ ID NO:19,
c) a sequence as shown in SEQ ID NO:20, and
d) a sequence which is at least 50% identical to any one of (a) to (c).

12. A method of stimulating cell growth and/or division, the method comprising contacting, or inserting into, an animal cell a polypeptide comprising a sequence selected from the group consisting of:
a) a sequence as shown in SEQ ID NO:21,
b) a sequence as shown in SEQ ID NO:22,
c) a sequence as shown in SEQ ID NO:23, and
d) a sequence which is at least 65% identical to any one of (a) to (c).

13. A method of stimulating cell growth and/or division, the method comprising contacting, or inserting into, an animal cell a polypeptide comprising a sequence selected from the group consisting of:
a) a sequence as shown in SEQ ID NO:24,
b) a sequence as shown in SEQ ID NO:25,
c) a sequence as shown in SEQ ID NO:26, and
d) a sequence which is at least 75% identical to any one of (a) to (c).

14. A method of stimulating cell growth and/or division, the method comprising contacting, or inserting into, an animal cell a polypeptide comprising a sequence selected from the group consisting of:
a) a sequence as shown in SEQ ID NO:27, and
b) a sequence which is at least 35% identical to a).

15. The method according to any one of the preceding claims, wherein the cell is a somatic cell.

16. The method according to claim 15, wherein the somatic cell is a mesenchymal cell.

17. The method according to claim 16, wherein the mesenchymal cell is selected from the group consisting of: chondrocytes and osteocytes.

18. The method according to any one of the preceding claims, wherein the polypeptide is provided by introducing into the cell an expression vector encoding the polypeptide.

19. The method according to any one of the preceding claims, wherein the cell is removed from an animal, cultured in vitro, transformed or transfected with a polynucleotide encoding the polypeptide and then placed back into an animal.

20. The method according to any one of claims 1 to 18, wherein the cell is transformed or transfected in vivo with a polynucleotide encoding the polypeptide.

21. A method of inhibiting cell growth and/or division, the method comprising contacting, or inserting into, an animal cell a compound which hybridizes to, and inhibits the translation of, a polynucleotide encoding a polypeptide selected from the group consisting of:
a) a sequence as shown in SEQ ID NO:1,
b) a sequence as shown in SEQ ID NO:2,
c) a sequence as shown in SEQ ID NO:3, and
d) a sequence which is at least 50% identical to any one of (a) to (c).

22. A method of inhibiting cell growth and/or division, the method comprising contacting, or inserting into, an animal cell a compound which hybridizes to, and inhibits the translation of, a polynucleotide encoding a polypeptide selected from the group consisting of:
a) a sequence as shown in SEQ ID NO:4,
b) a sequence as shown in SEQ ID NO:5,
c) a sequence as shown in SEQ ID NO:6, and
d) a sequence which is at least 70% identical to any one of (a) to (c).

23. A method of inhibiting cell growth and/or division, the method comprising contacting, or inserting into, an animal cell a compound which hybridizes to, and inhibits the translation of, a polynucleotide encoding a polypeptide selected from the group consisting of:
a) a sequence as shown in SEQ ID NO:7,
b) a sequence as shown in SEQ ID NO:8,
c) a sequence as shown in SEQ ID NO:9, and
d) a sequence which is at least 80% identical to any one of (a) to (c).

24. A method of inhibiting cell growth and/or division, the method comprising contacting, or inserting into, an animal cell a compound which hybridizes to, and inhibits the translation of, a polynucleotide encoding a polypeptide selected from the group consisting of:
a) a sequence as shown in SEQ ID NO:10,
b) a sequence as shown in SEQ ID NO:11,
c) a sequence as shown in SEQ ID NO:12, and
d) a sequence which is at least 85% identical to any one of (a) to (c).

25. A method of inhibiting cell growth and/or division, the method comprising contacting, or inserting into, an animal cell a compound which hybridizes to, and inhibits the translation of, a polynucleotide encoding a polypeptide selected from the group consisting of:
a) a sequence as shown in SEQ ID NO:13,
b) a sequence as shown in SEQ ID NO:14, and
c) a sequence which is at least 70% identical to any one of (a) or (b).

26. A method of inhibiting cell growth and/or division, the method comprising contacting, or inserting into, an animal cell a compound which hybridizes to, and inhibits the translation of, a polynucleotide encoding a polypeptide selected from the group consisting of:
a) a sequence as shown in SEQ ID NO:15,
b) a sequence as shown in SEQ ID NO:16, and
c) a sequence which is at least 50% identical to any one of (a) or (b).

27. A method of inhibiting cell growth and/or division, the method comprising contacting, or inserting into, an animal cell a compound which hybridizes to, and inhibits the translation of, a polynucleotide encoding a polypeptide selected from the group consisting of:
a) a sequence as shown in SEQ ID NO:17, and
b) a sequence which is at least 60% identical to a).

28. A method of inhibiting cell growth and/or division, the method comprising contacting, or inserting into, an animal cell a compound which hybridizes to, and inhibits the translation of, a polynucleotide encoding a polypeptide selected from the group consisting of:
a) a sequence as shown in SEQ ID NO:18,
b) a sequence as shown in SEQ ID NO:19,
c) a sequence as shown in SEQ ID NO:20, and
d) a sequence which is at least 50% identical to any one of (a) to (c).

29. A method of inhibiting cell growth and/or division, the method comprising contacting, or inserting into, an animal cell a compound which hybridizes to, and inhibits the translation of, a polynucleotide encoding a polypeptide selected from the group consisting of:
a) a sequence as shown in SEQ ID NO:21,
b) a sequence as shown in SEQ ID NO:22,
c) a sequence as shown in SEQ ID NO:23, and
d) a sequence which is at least 65% identical to any one of (a) to (c).

30. A method of inhibiting cell growth and/or division, the method comprising contacting, or inserting into, an animal cell a compound which hybridizes to, and inhibits the translation of, a polynucleotide encoding a polypeptide selected from the group consisting of:
a) a sequence as shown in SEQ ID NO:24,
b) a sequence as shown in SEQ ID NO:25,
c) a sequence as shown in SEQ ID NO:26, and
d) a sequence which is at least 75% identical to any one of (a) to (c).

31. A method of inhibiting cell growth and/or division, the method comprising contacting, or inserting into, an animal cell a compound which hybridizes to, and inhibits the translation of, a polynucleotide encoding a polypeptide selected from the group consisting of:
a) a sequence as shown in SEQ ID NO:27, and
b) a sequence which is at least 35% identical to a).

32. A method of identifying an agent that modulates the activity of a polypeptide that stimulates animal cell growth and/or division, the method comprising
i) exposing the polypeptide to a candidate agent, and
ii) assessing the ability of the candidate agent to modulate the ability of the polypeptide to stimulate cell growth and/or division,
wherein the polypeptide has a sequence selected from the group consisting of:
a) a sequence as shown in SEQ ID NO:1,
b) a sequence as shown in SEQ ID NO:2,
c) a sequence as shown in SEQ ID NO:3, and
d) a sequence which is at least 50% identical to any one of (a) to (c).

33. A method of identifying an agent that modulates the activity of a polypeptide that stimulates animal cell growth and/or division, the method comprising
i) exposing the polypeptide to a candidate agent, and
ii) assessing the ability of the candidate agent to modulate the ability of the polypeptide to stimulate cell growth and/or division,
wherein the polypeptide has a sequence selected from the group consisting of:
a) a sequence as shown in SEQ ID NO:4,
b) a sequence as shown in SEQ ID NO:5,
c) a sequence as shown in SEQ ID NO:6, and
d) a sequence which is at least 70% identical to any one of (a) to (c).

34. A method of identifying an agent that modulates the activity of a polypeptide that stimulates animal cell growth and/or division, the method comprising
i) exposing the polypeptide to a candidate agent, and
ii) assessing the ability of the candidate agent to modulate the ability of the polypeptide to stimulate cell growth and/or division,
wherein the polypeptide has a sequence selected from the group consisting of:
a) a sequence as shown in SEQ ID NO:7,
b) a sequence as shown in SEQ ID NO:8,
c) a sequence as shown in SEQ ID NO:9, and
d) a sequence which is at least 80% identical to any one of (a) to (c).

35. A method of identifying an agent that modulates the activity of a polypeptide that stimulates animal cell growth and/or division, the method comprising
i) exposing the polypeptide to a candidate agent, and
ii) assessing the ability of the candidate agent to modulate the ability of the polypeptide to stimulate cell growth and/or division,
wherein the polypeptide has a sequence selected from the group consisting of:
a) a sequence as shown in SEQ ID NO:10,
b) a sequence as shown in SEQ ID NO:11,
c) a sequence as shown in SEQ ID NO:12, and
d) a sequence which is at least 85% identical to any one of (a) to (c).

36. A method of identifying an agent that modulates the activity of a polypeptide that stimulates animal cell growth and/or division, the method comprising
i) exposing the polypeptide to a candidate agent, and
ii) assessing the ability of the candidate agent to modulate the ability of the polypeptide to stimulate cell growth and/or division,
wherein the polypeptide has a sequence selected from the group consisting of:
a) a sequence as shown in SEQ ID NO:13,
b) a sequence as shown in SEQ ID NO:14, and
c) a sequence which is at least 70% identical to any one of (a) or (b).

37. A method of identifying an agent that modulates the activity of a polypeptide that stimulates animal cell growth and/or division, the method comprising
i) exposing the polypeptide to a candidate agent, and
ii) assessing the ability of the candidate agent to modulate the ability of the polypeptide to stimulate cell growth and/or division,
wherein the polypeptide has a sequence selected from the group consisting of:
a) a sequence as shown in SEQ ID NO:15,
b) a sequence as shown in SEQ ID NO:16, and
c) a sequence which is at least 50% identical to any one of (a) or (b).

38. A method of identifying an agent that modulates the activity of a polypeptide that stimulates animal cell growth and/or division, the method comprising
i) exposing the polypeptide to a candidate agent, and
ii) assessing the ability of the candidate agent to modulate the ability of the polypeptide to stimulate cell growth and/or division, wherein the polypeptide has a sequence selected from the group consisting of:
a) a sequence as shown in SEQ ID NO:17, and
b) a sequence which is at least 60% identical to a).

39. A method of identifying an agent that modulates the activity of a polypeptide that stimulates animal cell growth and/or division, the method comprising
i) exposing the polypeptide to a candidate agent, and
ii) assessing the ability of the candidate agent to modulate the ability of the polypeptide to stimulate cell growth and/or division,
wherein the polypeptide has a sequence selected from the group consisting of:
a) a sequence as shown in SEQ ID NO:18,
b) a sequence as shown in SEQ ID NO:19,
c) a sequence as shown in SEQ ID NO:20, and
d) a sequence which is at least 50% identical to any one of (a) to (c).

40. A method of identifying an agent that modulates the activity of a polypeptide that stimulates animal cell growth and/or division, the method comprising
i) exposing the polypeptide to a candidate agent, and
ii) assessing the ability of the candidate agent to modulate the ability of the polypeptide to stimulate cell growth and/or division,
wherein the polypeptide has a sequence selected from the group consisting of:
a) a sequence as shown in SEQ ID NO:21,
b) a sequence as shown in SEQ ID NO:22,
c) a sequence as shown in SEQ ID NO:23, and
d) a sequence which is at least 65% identical to any one of (a) to (c).

41. A method of identifying an agent that modulates the activity of a polypeptide that stimulates animal cell growth and/or division, the method comprising
i) exposing the polypeptide to a candidate agent, and
ii) assessing the ability of the candidate agent to modulate the ability of the polypeptide to stimulate cell growth and/or division,
wherein the polypeptide has a sequence selected from the group consisting of:
a) a sequence as shown in SEQ ID NO:24,
b) a sequence as shown in SEQ ID NO:25,
c) a sequence as shown in SEQ ID NO:26, and
d) a sequence which is at least 75% identical to any one of (a) to (c).

42. A method of identifying an agent that modulates the activity of a polypeptide that stimulates animal cell growth and/or division, the method comprising
i) exposing the polypeptide to a candidate agent, and
ii) assessing the ability of the candidate agent to modulate the ability of the polypeptide to stimulate cell growth and/or division,
wherein the polypeptide has a sequence selected from the group consisting of:
a) a sequence as shown in SEQ ID NO:27, and
b) a sequence which is at least 35% identical to a).

43. The method according to any one of claims 32 to 42, wherein the agent inhibits the ability of the polypeptide to stimulate cell growth and/or division.

44. The method according to any one of claims 32 to 42, wherein the agent enhances the ability of the polypeptide to stimulate cell growth and/or division.

45. The method according to any one of the preceding claims, wherein the animal cell is a mammalian cell.

46. A method of stimulating mesenchymal cell growth and/or division, the method comprising exposing animal mesenchymal cells to conditioned media, or an active fraction thereof, obtained from deer antler cartilage cells.

47. The method according to claim 46, wherein the deer antler cartilage cells are selected from the group consisting of: prechondrocytes, mature chondrocytes, hypertropic chondrocytes, or a combination thereof.

48. The method according to claim 46 or claim 47, wherein the method further comprises exposing the cells to a growth factor.

49. The method according to claim 48, wherein the growth factor is selected from the group consisting of: insulin-like growth factor (IGF-1), TGF-beta, fibroblast growth factor (FGF), vascular endothelial growth factor (VEGF), morphogenic bone factors, thyroid hormones (thyroxine), parathyroid hormone related protein (PTHrP), sex hormones, luteinizing hormone (LH) and prolactin.

50. A substantially purified polypeptide comprising a sequence selected from the group consisting of:
a) a sequence as shown in SEQ ID NO:1, and
b) a sequence which is at least 91% identical to a),
wherein the polypeptide is capable of stimulating animal cell growth and/or division.

51. The polypeptide according to claim 50, wherein the sequence is least 95% identical to a).

52. The polypeptide according to claim 50, wherein the sequence is least 99% identical to a).

53. A substantially purified polypeptide comprising a sequence selected from the group consisting of:
a) a sequence as shown in SEQ ID NO:4, and
b) a sequence which is at least 99% identical to a),
wherein the polypeptide has a biological activity selected from the group consisting of: stimulating animal cell growth and/or division, or a structural component of extracellular matrix.

54. A substantially purified polypeptide comprising a sequence selected from the group consisting of:
a) a sequence as shown in SEQ ID NO:7, and
b) a sequence which is at least 99% identical to a),
wherein the polypeptide has a biological activity selected from the group consisting of: stimulating animal cell growth and/or division, or subunit involved in protein synthesis.

55. A substantially purified polypeptide comprising a sequence selected from the group consisting of:
a) a sequence as shown in SEQ ID NO:13, and
b) a sequence which is at least 90% identical to a),
wherein the polypeptide has a biological activity selected from the group consisting of: stimulating animal cell growth and/or division, or altering chromatin structure.

56. A substantially purified polypeptide comprising a sequence selected from the group consisting of:
a) a sequence as shown in SEQ ID NO:15, and
b) a sequence which is at least 99% identical to a),
wherein the polypeptide has a biological activity selected from the group consisting of: stimulating animal cell growth and/or division, or regulating cell migration.

57. A substantially purified polypeptide comprising a sequence selected from the group consisting of:
a) a sequence as shown in SEQ ID NO:18, and
b) a sequence which is at least 91% identical to a),
wherein the polypeptide has a biological activity selected from the group consisting of: stimulating animal cell growth and/or division, or responses to cell stress.

58. A substantially purified polypeptide comprising a sequence selected from the group consisting of:
a) a sequence as shown in SEQ ID NO:21, and
b) a sequence which is at least 96% identical to a),
wherein the polypeptide has a biological activity selected from the group consisting of: stimulating animal cell growth and/or division, or a component of connective tissue, or collagen fibrillogenesis.

59. A substantially purified polypeptide comprising a sequence selected from the group consisting of:
a) a sequence as shown in SEQ ID NO:24, and
b) a sequence which is at least 98% identical to a),
wherein the polypeptide has a biological activity selected from the group consisting of: stimulating animal cell growth and/or division, or a component of collagen.

60. A fusion protein comprising a polypeptide according to any one of claims 50 to 59.

61. An isolated polynucleotide encoding a polypeptide according to any one of claims 50 to 60.

62. The polynucleotide according to claim 61 comprising a sequence according to any one of SEQ ID NO:28, 29, 31 to 33, or 35 to 38.

63. An isolated polynucleotide comprising a sequence provided as SEQ ID NO:30.

64. An isolated polynucleotide comprising a sequence provided as SEQ ID NO:34.

65. An antisense polynucleotide which hybridizes under high stringency conditions to a polynucleotide according to any one of claims 61 to 64.

66. A vector comprising the polynucleotide according to any one of claims 61 to 65.

67. The vector according to claim 66, wherein the polynucleotide is operably linked to a promoter.

68. A host cell transfected or transformed with a vector according to claim 66 or claim 67.

69. The host cell of claim 68 which is a mammalian cell.

70. A method according to any on of claims 14, 31, 42 and 46 wherein the increased cell division and/or matrix gene expression by chondrocytes results from the action of transthyretin.
